(19) United States
(12) Patent Application Publication
(10) Pub. No.: US 2026/0137342 A1
(43) Pub. Date: May 21, 2026

(54) EEG Glasses (Electroencephalographic Eyewear)

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 19/450,101

(22) Filed: Jan. 15, 2026

Related U.S. Application Data
(63) Continuation-in-part of application No. 19/020,737, filed on Jan. 14, 2025.
(63) Continuation-in-part of application No. 18/944,224, filed on Nov. 12, 2024.
(63) Continuation-in-part of application No. 18/902,821, filed on Sep. 30, 2024.
Application No. 19/020,737 is a continuation-in-part of application No. 18/944,224, filed on Nov. 12, 2024.
Application No. 18/944,224 is a continuation-in-part of application No. 18/902,821, filed on Sep. 30, 2024.
Application No. 18/902,821 is a continuation-in-part of application No. 18/748,059, filed on Jun. 19, 2024, now Pat. No. 12,201,427.
Application No. 18/748,059 is a continuation-in-part of application No. 18/411,540, filed on Jan. 12, 2024, now Pat. No. 12,310,749.
Application No. 18/944,224 is a continuation-in-part of application No. 18/902,821, filed on Sep. 30, 2024.
Application No. 18/902,821 is a continuation-in-part of application No. 18/748,059, filed on Jun. 19, 2024, now Pat. No. 12,201,427.
Application No. 18/748,059 is a continuation-in-part of application No. 18/411,540, filed on Jan. 12, 2024, now Pat. No. 12,310,749.
Application No. 18/411,540 is a continuation-in-part of application No. 18/219,684, filed on Jul. 9, 2023, now abandoned.
Application No. 18/748,059 is a continuation-in-part of application No. 18/411,540, filed on Jan. 12, 2024, now Pat. No. 12,310,749.
Application No. 18/411,540 is a continuation-in-part of application No. 18/219,684, filed on Jul. 9, 2023, now abandoned.
Application No. 18/411,540 is a continuation-in-part of application No. 18/219,684, filed on Jul. 9, 2023, now abandoned.
Application No. 18/219,684 is a continuation-in-part of application No. 17/714,988, filed on Apr. 6, 2022, now Pat. No. 11,850,052.
Application No. 17/714,988 is a continuation-in-part of application No. 16/838,541, filed on Apr. 2, 2020, now abandoned.
Application No. 17/714,988 is a continuation-in-part of application No. 17/665,086, filed on Feb. 4, 2022, now Pat. No. 11,662,819.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/136,117 is a continuation-in-part of application No. 16/838,541, filed on Apr. 2, 2020, now abandoned.
Application No. 16/838,541 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/554,029, filed on Aug. 28, 2019, now abandoned.
Application No. 16/554,029 is a continuation-in-part of application No. 16/554,029, filed on Aug. 28, 2019, now abandoned.
Application No. 16/554,029 is a continuation-in-part of application No. 15/236,401, filed on Aug. 13, 2016, now abandoned.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 16/568,580 is a continuation-in-part of application No. 15/963,061, filed on Apr. 25, 2018, now Pat. No. 10,772,559.
Application No. 15/963,061 is a continuation-in-part of application No. 16/022,987, filed on Jun. 29, 2018, now Pat. No. 11,172,859.
Application No. 16/022,987 is a continuation-in-part of application No. 15/136,948, filed on Apr. 24, 2016, now Pat. No. 10,234,942.
Application No. 15/963,061 is a continuation-in-part of application No. 15/464,349, filed on Mar. 21, 2017, now Pat. No. 9,968,297.
Application No. 15/464,349 is a continuation-in-part of application No. 15/136,948, filed on Apr. 24, 2016, now Pat. No. 10,234,942.
Application No. 15/136,948 is a continuation-in-part of application No. 14/562,719, filed on Dec. 7, 2014, now Pat. No. 10,130,277.
Application No. 14/562,719 is a continuation-in-part of application No. 14/330,649, filed on Jul. 14, 2014, now abandoned.
Application No. 15/236,401 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
Application No. 14/330,649 is a continuation-in-part of application No. 13/797,955, filed on Mar. 12, 2013, now Pat. No. 9,456,916.
Application No. 13/797,955 is a continuation-in-part of application No. 13/523,739, filed on Jun. 14, 2012, now Pat. No. 9,042,596.
(60) Provisional application No. 61/729,494, filed on Nov. 23, 2012.
(60) Provisional application No. 61/932,517, filed on Jan. 28, 2014.
(60) Provisional application No. 61/932,517, filed on Jan. 28, 2014.
(60) Provisional application No. 61/939,244, filed on Feb. 12, 2014.
(60) Provisional application No. 62/017,615, filed on Jun. 26, 2014.
(60) Provisional application No. 62/089,696, filed on Dec. 9, 2014.
(60) Provisional application No. 62/160,172, filed on May 12, 2015.
(60) Provisional application No. 62/169,661, filed on Jun. 2, 2015.
(60) Provisional application No. 62/303,126, filed on Mar. 3, 2016.
(60) Provisional application No. 62/322,594, filed on Apr. 14, 2016.
(60) Provisional application No. 62/430,667, filed on Dec. 6, 2016.
(60) Provisional application No. 62/791,838, filed on Jan. 13, 2019.
(60) Provisional application No. 62/796,901, filed on Jan. 25, 2019.
(60) Provisional application No. 62/851,904, filed on May 23, 2019.
(60) Provisional application No. 62/972,692, filed on Feb. 11, 2020.
(60) Provisional application No. 62/972,692, filed on Feb. 11, 2020.

(51) Int. Cl. A61B 5/00; A61B 5/369

(52) U.S. Cl. CPC A61B 5/6803; A61B 5/0006; A61B 5/369

(57) ABSTRACT

Disclosed herein is a pair of EEG glasses or other electroencephalographic eyewear comprising an eyewear frame with a frontpiece and two sidepieces (e.g. temples), wherein there is an arcuate arm which extends out from the sidepiece toward the person's head and an electrode on the arcuate protrusion. The electrode collects data concerning the person's brain activity.

### CROSS-REFERENCE TO RELATED APPLICATIONS

This patent application is a continuation-in-part of U.S. patent application Ser. No. 19/020,737 filed on Jan. 14, 2025. This patent application is a continuation-in-part of U.S. patent application Ser. No. 18/944,224 filed on Nov. 12, 2024. This patent application is a continuation-in-part of U.S. patent application Ser. No. 18/902,821 filed on Sep. 30, 2024.

U.S. patent application Ser. No. 19/020,737 was a continuation-in-part of U.S. patent application Ser. No. 18/944,224 filed on Nov. 12, 2024. U.S. patent application Ser. No. 19/020,737 was a continuation-in-part of U.S. patent application Ser. No. 18/902,821 filed on Sep. 30, 2024. U.S. patent application Ser. No. 19/020,737 was a continuation-in-part of U.S. patent application Ser. No. 18/748,059 filed on Jun. 19, 2024. U.S. patent application Ser. No. 19/020,737 was a continuation-in-part of U.S. patent application Ser. No. 18/411,540 filed on Jan. 12, 2024.

U.S. patent application Ser. No. 18/944,224 was a continuation-in-part of U.S. patent application Ser. No. 18/902,821 filed on Sep. 30, 2024. U.S. patent application Ser. No. 18/944,224 was a continuation-in-part of U.S. patent application Ser. No. 18/748,059 filed on Jun. 19, 2024. U.S. patent application Ser. No. 18/944,224 was a continuation-in-part of U.S. patent application Ser. No. 18/411,540 filed on Jan. 12, 2024. U.S. patent application 18/944,224 was a continuation-in-part of U.S. patent application Ser. No. 18/219,684 filed on Jul. 9, 2023.

U.S. patent application Ser. No. 18/902,821 was a continuation-in-part of U.S. patent application Ser. No. 18/748,059 filed on Jun. 19, 2024. U.S. patent application Ser. No. 18/902,821 was a continuation-in-part of U.S. patent application Ser. No. 18/411,540 filed on Jan. 12, 2024. U.S. patent application Ser. No. 18/902,821 was a continuation-in-part of U.S. patent application Ser. No. 18/219,684 filed on Jul. 9, 2023.

U.S. patent application Ser. No. 18/748,059 was a continuation-in-part of U.S. patent application Ser. No. 18/411,540 filed on Jan. 12, 2024. U.S. patent application Ser. No. 18/748,059 was a continuation-in-part of U.S. patent application Ser. No. 18/219,684 filed on Jul. 9, 2023. U.S. patent application Ser. No. 18/411,540 was a continuation-in-part of U.S. patent application Ser. No. 18/219,684 filed on Jul. 9, 2023. U.S. patent application Ser. No. 18/219,684 was a continuation-in-part of U.S. patent application Ser. No. 17/714,988 filed on Apr. 6, 2022. U.S. patent application Ser. No. 18/219,684 was a continuation-in-part of U.S. patent application Ser. No. 16/838,541 filed on Apr. 2, 2020.

U.S. patent application Ser. No. 17/714,988 was a continuation-in-part of U.S. patent application Ser. No. 17/665,086 filed on Feb. 4, 2022. U.S. patent application Ser. No. 17/714,988 was a continuation-in-part of U.S. patent application Ser. No. 17/136,117 filed on Dec. 29, 2020. U.S. patent application Ser. No. 17/714,988 was a continuation-in-part of U.S. patent application Ser. No. 16/554,029 filed on Aug. 28, 2019. U.S. patent application Ser. No. 17/665,086 was a continuation-in-part of U.S. patent application Ser. No. 17/136,117 filed on Dec. 29, 2020. U.S. patent application Ser. No. 17/665,086 was a continuation-in-part of U.S. patent application Ser. No. 16/554,029 filed on Aug. 28, 2019.

U.S. patent application Ser. No. 17/136,117 was a continuation-in-part of U.S. patent application Ser. No. 16/838,541 filed on Apr. 2, 2020. U.S. patent application Ser. No. 17/136,117 claimed the priority benefit of U.S. provisional patent application 62972692 filed on Feb. 11, 2020. U.S. patent application Ser. No. 17/136,117 was a continuation-in-part of U.S. patent application Ser. No. 16/737,052 filed on Jan. 8, 2020. U.S. patent application Ser. No. 17/136,117 was a continuation-in-part of U.S. patent application Ser. No. 16/568,580 filed on Sep. 12, 2019. U.S. patent application Ser. No. 17/136,117 was a continuation-in-part of U.S. patent application Ser. No. 16/554,029 filed on Aug. 28, 2019.

U.S. patent application Ser. No. 16/838,541 claimed the priority benefit of U.S. provisional patent application 62972692 filed on Feb. 11, 2020. U.S. patent application Ser. No. 16/838,541 was a continuation-in-part of U.S. patent application Ser. No. 16/554,029 filed on Aug. 28, 2019. U.S. patent application Ser. No. 16/838,541 claimed the priority benefit of U.S. provisional patent application 62851917 filed on May 23, 2019. U.S. patent application Ser. No. 16/838,541 claimed the priority benefit of U.S. provisional patent application 62837712 filed on Apr. 23, 2019. U.S. patent application Ser. No. 16/838,541 was a continuation-in-part of U.S. patent application Ser. No. 15/236,401 filed on Aug. 13, 2016. U.S. patent application Ser. No. 16/,737,052 was a continuation-in-part of U.S. patent application Ser. No. 16/568,580 filed on Sep. 12, 2019. U.S. patent application Ser. No. 16/737,052 was a continuation-in-part of U.S. patent application Ser. No. 15/963,061 filed on Apr. 25, 2018. U.S. patent application Ser. No. 16/568,580 was a continuation-in-part of U.S. patent application Ser. No. 15/963,061 filed on Apr. 25, 2018.

U.S. patent application Ser. No. 16554029 claimed the priority benefit of U.S. provisional patent application 62851904 filed on May 23, 2019. U.S. patent application Ser. No. 16/554,029 claimed the priority benefit of U.S. provisional patent application 62796901 filed on Jan. 25, 2019. U.S. patent application Ser. No. 16/554,029 claimed the priority benefit of U.S. provisional patent application 62791838 filed on Jan. 13, 2019. U.S. patent application Ser. No. 16/554,029 was a continuation-in-part of U.S. patent application Ser. No. 16/022,987 filed on Jun. 29, 2018. U.S. patent application Ser. No. 16/022,987 was a continuation-in-part of U.S. patent application Ser. No. 15/136,948 filed on Apr. 24, 2016. U.S. patent application Ser. No. 15/963,061 was a continuation-in-part of U.S. patent application Ser. No. 15/464,349 filed on Mar. 21, 2017.

U.S. patent application Ser. No. 15/464,349 claimed the priority benefit of U.S. provisional patent application 62430667 filed on Dec. 6, 2016. U.S. patent application Ser. No. 15/464,349 was a continuation-in-part of U.S. patent application Ser. No. 15/136,948 filed on Apr. 24, 2016. U.S. patent application Ser. No. 15/464,349 was a continuation-in-part of U.S. patent application Ser. No. 14/562,719 filed on Dec. 7, 2014. U.S. patent application Ser. No. 15/464,349 was a continuation-in-part of U.S. patent application Ser. No. 14/330.649 filed on Jul. 14, 2014. U.S. patent application Ser. No. 15/236,401 was a continuation-in-part of U.S. patent application Ser. No. 15/136,948 filed on Apr. 24, 2016. U.S. patent application Ser. No. 15/236,401 was a continuation-in-part of U.S. patent application Ser. No. 14/599,522 filed on Jan. 18, 2015.

U.S. patent application Ser. No. 15/136,948 claimed the priority benefit of U.S. provisional patent application 62322594 filed on Apr. 14, 2016. U.S. patent application Ser. No. 15/136,948 claimed the priority benefit of U.S. provisional patent application 62303126 filed on Mar. 3, 2016. U.S. patent application Ser. No. 15/136,948 claimed the priority benefit of U.S. provisional patent application 62169661 filed on Jun. 2, 2015. U.S. patent application Ser. No. 15/136,948 claimed the priority benefit of U.S. provisional patent application 62160172 filed on May, 12, 2015. U.S. patent application Ser. No. 15/136,948 was a continuation-in-part of U.S. patent application Ser. No. 14/599,522 filed on Jan. 18, 2015.

U.S. patent application Ser. No. 14/599,522 claimed the priority benefit of U.S. provisional patent application 62089696 filed on Dec. 9, 2014. U.S. patent application Ser. No. 14/599,522 was a continuation-in-part of U.S. patent application Ser. No. 14/562,719 filed on Dec. 7, 2014. U.S. patent application Ser. No. 14/599,522 claimed the priority benefit of U.S. provisional patent application 62017615 filed on Jun. 26, 2014. U.S. patent application Ser. No. 14/599,522 claimed the priority benefit of U.S. provisional patent application 61939244 filed on Feb. 12, 2014. U.S. patent application Ser. No. 14/599,522 claimed the priority benefit of U.S. provisional patent application 61932517 filed on Jan. 28, 2014.

U.S. patent application Ser. No. 14/562,719 claimed the priority benefit of U.S. provisional patent application 61932517 filed on Jan. 28, 2014. U.S. patent application Ser. No. 14/330,649 was a continuation-in-part of U.S. patent application Ser. No. 13/797,955 filed on Mar. 12, 2013. U.S. patent application Ser. No. 14/330,649 was a continuation-in-part of U.S. patent application Ser. No. 13/523,739 filed on Jun. 14, 2012. U.S. patent application Ser. No. 13/797,955 claimed the priority benefit of U.S. provisional patent application 61729494 filed on Nov. 23, 2012.

The entire contents of these applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to wearable EEG (electroencephalographic) monitoring devices.

### INTRODUCTION

There are numerous potential applications for incorporating electrodes (e.g. electrical brain activity sensors) into devices which are worn on a person's head. Such devices can record brainwave activity (e.g. electroencephalographic data) for use as a BCI (Brain-to-Computer Interface) for communication. Such devices can also be used as a device to monitor biometric parameters to improve a person's health and/or detect adverse heath events (e.g. seizures, strokes, or heart attacks). Since eyeglasses are already a socially-accepted form for a head-worn device, incorporating electrodes (e.g. EEG sensors) into eyeglasses can be an advantageous form of device for these applications. However, traditional eyeglasses were designed to hold lenses in front of a person's eyes, not to hold electrodes on key areas of a person's head. For example, there are air gaps between traditional eyeglass frames and a person's temples or the sides of a person's forehead. Disclosed herein are innovative designs for eyeglasses and dry electrodes which can hold electrodes on key areas of a person's head better than traditional eyeglasses.

### REVIEW OF THE RELEVANT ART

In the patent literature, U.S. Patent Application Publication 20140347265 (Aimone et al., Nov. 27, 2014, “Wearable Computing Apparatus and Method”) and U.S. Pat. No. 10,365,716 (Aimone et al., Jul. 30, 2019, “Wearable Computing Apparatus and Method”) disclose a method performed by a wearable eyeglass frame with at least one EOG sensor and at least one EEG sensor. In one example, each sensor 110 may include EOG functionality in order to use EOG to measure eye properties. FIGS. 40-45 show sketches of eyeglasses with biosignal sensors which may be EOG or EEG sensors. Also, U.S. Patent Application Publication 20190101977 (Armstrong-Muntner et al., Apr. 4, 2019, “Monitoring a User of a Head-Wearable Electronic Device”) and U.S. Pat. No. 10,809,796 (Armstrong-Muntner et al., Oct. 20, 2020, “Monitoring a User of a Head-Wearable Electronic Device”) disclose systems, methods, and computer-readable media for monitoring a user of a head-wearable electronic device with multiple light-sensing assemblies.

Further, U.S. Patent Application Publication 20200375524 (Aminifar et al., Dec. 3, 2020, “A Wearable System for Real-Time Detection of Epileptic Seizures”) and U.S. Pat. No. 12,419,566 (Aminifar et al., Sep. 23, 2025, “A Wearable System for Real-Time Detection of Epileptic Seizures”) disclose a wearable system for epileptic seizure detection including an eyeglasses frame, with a left arm and a right arm configured to rest over the ears of an intended person wearing the eyeglasses, a first pair of electrodes located in the left arm, and a second pair of electrodes located in the right arm.

In the non-patent literature, Sopic et al., 2018, “e-Glass: A Wearable System for Real-Time Detection of Epileptic Seizures,” 2018 IEEE International Symposium on Circuits and Systems (ISCAS), Florence, Italy, 2018, p. 1-5, discloses e-Glass, a wearable system based on four electroencephalogram (EEG) electrodes for the detection of epileptic seizures. Also, Vourvopoulos et al, 2019, “EEGlass: An EEG-Eyeware Prototype for Ubiquitous Brain-Computer Interaction”, Adjunct Proceedings of the 2019 ACM International Joint Conference on Pervasive and Ubiquitous Computing and Proceedings of the 2019 ACM International Symposium on Wearable Computers, p. 647-652, discloses EEGlass, an early wearable prototype comprised of plastic eyewear frames for approximating the form factor of a modern HMD. EEGlass is equipped with a set of EEG electrodes at the contact points with the skull for unobtrusively collecting data related to the activity of the human brain.

Also, Kosmyna et al., 2019, “AttentivU: A Wearable Pair of EEG and EOG Glasses for Real-Time Physiological Processing,” 2019 IEEE 16th International Conference on Wearable and Implantable Body Sensor Networks (BSN), Chicago, IL, USA, 2019, p. 1-4, discloses AttentivU, a device using both EEG and EOG for real-time monitoring of physiological data. The device is designed as a socially acceptable pair of glasses with dry silver electrodes. Also, Ban et al., 2022, “Advances in Materials, Sensors, and Integrated Systems for Monitoring Eye Movements,” Biosensors, 2022, 12, 1039, outlines a systematic summary of the latest research on various materials, sensors, and integrated systems for monitoring eye movements and enabling human-machine interfaces.

Further, Frey et al., 2025, “GAPses: Versatile Smart Glasses for Comfortable and Fully-Dry Acquisition and Parallel Ultra-Low-Power Processing of EEG and EOG,” in IEEE Transactions on Biomedical Circuits and Systems, vol. 19, no. 3, p. 616-628, June 2025, discloses GAPSES, a novel smart glasses platform designed for unobtrusive, comfortable, and secure acquisition and processing of electroencephalography (EEG) and electrooculography (EOG) signals. The glasses have a sleek frame design with soft dry electrodes. Finally, Zanetti et al., 2025, “EEG glasses for real-time brain electrical activity monitoring,” Nature, Scientific Reports, 15, 43574, 2025, discloses e-Glass, a state-of-the-art smart wearable device that enables unobtrusive real-time electroencephalography (EEG) monitoring.

### SUMMARY OF THE INVENTION

This invention is EEG glasses or other electroencephalographic eyewear comprising an eyewear frame with a frontpiece and two sidepieces (e.g. temples), wherein there is an arcuate protrusion which extends out from the sidepiece toward the person's head and an electrode (or other electromagnetic energy sensor) on the arcuate protrusion. The electrode collects data concerning the person's brain activity. The eyewear can further comprise an energy source (e.g. battery), a data processor; and a data transmitter and/or receiver. In an example, there can be a spring between the protrusion and the sidepiece. These EEG glasses can hold electrodes on key areas of a person's head better than traditional eyeglasses.

### BRIEF INTRODUCTION TO THE FIGURES

FIGS. 1 and 2 show virtual lines with respect to eyewear designs which are later used to specify designs more precisely.

FIG. 3 shows EEG glasses with a sinusoidal flexible protrusion on a side section.

FIG. 4 shows EEG glasses with an elliptical or oval protrusion on a side section.

FIG. 5 shows EEG glasses with a cylindrical or rectangular protrusion on a side section.

FIG. 6 shows EEG glasses with multiple sinusoidal protrusions on a side section.

FIG. 7 shows EEG glasses with an arcuate tensile protrusion on a side section.

FIG. 8 shows EEG glasses with a spring-attached tensile protrusion on a side section.

FIG. 9 shows EEG glasses with a sinusoidal tensile protrusion on a side section.

FIG. 10 shows EEG glasses with a spring-attached sinusoidal tensile protrusion on a side section.

FIGS. 11 and 12 show how an arcuate tensile protrusion on EEG glasses can be adjusted.

FIG. 13 shows EEG glasses with a folded and/or pleated protrusion on a side section.

FIG. 14 shows EEG glasses with a spring-attached pivoting protrusion on a side section.

FIG. 15 shows EEG glasses with a wedge-shaped flexible protrusion on a side section.

FIG. 16 shows EEG glasses with an adjustable wedge-shaped flexible protrusion on a side section.

FIGS. 17 and 18 show two views of EEG glasses with an upward wave on a side section.

FIGS. 19 and 20 show two views of EEG glasses with an upward wave and flexible protrusion on a side section.

FIGS. 21 and 22 show two views of first example of EEG glasses with two upward waves on a side section.

FIGS. 23 and 24 show two views of second example of EEG glasses with two upward waves on a side section.

FIGS. 25 and 26 show two views of EEG glasses with two upward waves and an inward wave on a side section.

FIGS. 27 and 28 show two views of EEG glasses with two upward waves and a prong on a side section.

FIGS. 29 and 30 show two views of EEG glasses with a bifurcation on a side section.

FIGS. 31 and 32 show two views of EEG glasses with a forehead-spanning curve.

FIGS. 33 and 34 show two views of EEG glasses with an upward and inward curving fin or wedge.

FIG. 35 shows EEG glasses with an arcuate bifurcating wave onto a person's forehead and/or temple.

FIG. 36 shows EEG glasses with an arcuate upward wave onto a person's forehead and/or temple.

FIGS. 37 and 38 show how EEG glasses can be used to measure and/or modify a person's food consumption.

FIGS. 39 and 40 show two views of a first example of EEG glasses with a loop that moves onto a person's forehead.

FIGS. 41 and 42 show two views of a second example of EEG glasses with a loop that moves onto a person's forehead.

FIGS. 43 and 44 show two views of a first example of EEG glasses with a forward-backward loop over a person's forehead.

FIGS. 45 and 46 show two views of a second example of EEG glasses with a forward-backward loop over a person's forehead.

FIGS. 47 and 48 show two views EEG glasses with a side section bifurcation and a forward-backward loop over a person's forehead.

### DETAILED DESCRIPTION OF THE FIGURES

In an example, EEG glasses or other electroencephalographic eyewear can comprise: an eyewear frame, wherein the eyewear frame further comprises a frontpiece which is configured to span the front of a person's head, a first sidepiece which is configured to span from a first ear to the frontpiece, and a second sidepiece which is configured to span from a second ear to the frontpiece; an arcuate tensile protrusion which is part of, or attached to, a selected sidepiece; an electrode or other electromagnetic energy sensor on the protrusion which collects data concerning brain activity; an energy source; a data processor; and a data transmitter and/or receiver.

In an example, there can be a spring between the protrusion and the selected sidepiece. In an example, one end of the protrusion can be attached to the sidepiece and the other end of the protrusion can be free to extend out from the sidepiece. In an example, a posterior end of the protrusion can be attached to the sidepiece and an anterior end of the protrusion can be free to extend out from the sidepiece. In an example, both ends of the protrusion can be attached to the sidepiece.

In an example, the protrusion can have a first configuration which extends a first distance out from the selected sidepiece toward a person's head, the protrusion can have a second configuration which extends a second distance out from the selected sidepiece toward the person's head, and the second distance can be greater than the first distance. In an example, the protrusion can be changed from the first configuration to the second configuration by moving one or both of the ends of the protrusion closer to each other, causing the protrusion to bulge out towards toward the person's head. In an example, one or both of the ends of the protrusion can slide along the sidepiece. In an example, one or both of the ends of the protrusion can slide along a track or channel on the sidepiece.

In an example, EEG glasses or other electroencephalographic eyewear can comprise: an eyewear frame which further comprises a front section which is configured to span the front of a person's head, a first side section which is configured to span from a first ear to the front section, and a second side section which is configured to span from a second ear to the front section; an arcuate protrusion which is part of, or attached to, a selected side section; an electromagnetic energy sensor which collects data concerning electromagnetic brain activity, wherein the protrusion is configured to hold the electromagnetic energy sensor on the person's head; an energy source; a data processor; and a data transmitter and/or receiver.

In an example, there can be a spring between the protrusion and the selected sidepiece. In an example, one end of the protrusion can be attached to the sidepiece and the other end of the protrusion can be free to extend out from the sidepiece. In an example, a posterior end of the protrusion can be attached to the sidepiece and an anterior end of the protrusion can be free to extend out from the sidepiece. In an example, both ends of the protrusion can be attached to the sidepiece.

In an example, the protrusion can haves a first configuration which extends a first distance out from the selected sidepiece toward a person's head, the protrusion can have a second configuration which extends a second distance out from the selected sidepiece toward the person's head, and the second distance can be greater than the first distance. In an example, the protrusion can be changed from the first configuration to the second configuration by moving one or both of the ends of the protrusion closer to each other, causing the protrusion to bulge out towards toward the person's head. In an example, one or both of the ends of the protrusion can slide along the sidepiece. In an example, one or both of the ends of the protrusion can slide along a track or channel on the sidepiece.

FIG. 1 shows a left-side view of an example of a wearable brain activity monitor comprising a head-worn sensor-positioning member 102 which is configured to position a plurality of electrodes or other brain activity sensors, including 103, at selected locations on the wearer's head 101. This monitor further comprises control unit 104. In an example, the monitor can be symmetric, with a similarly-configured sensor-positioning member on the right side of the wearer's head. In an alternative example, the monitor can asymmetric, with no sensor-positioning member on the wearer's right side.

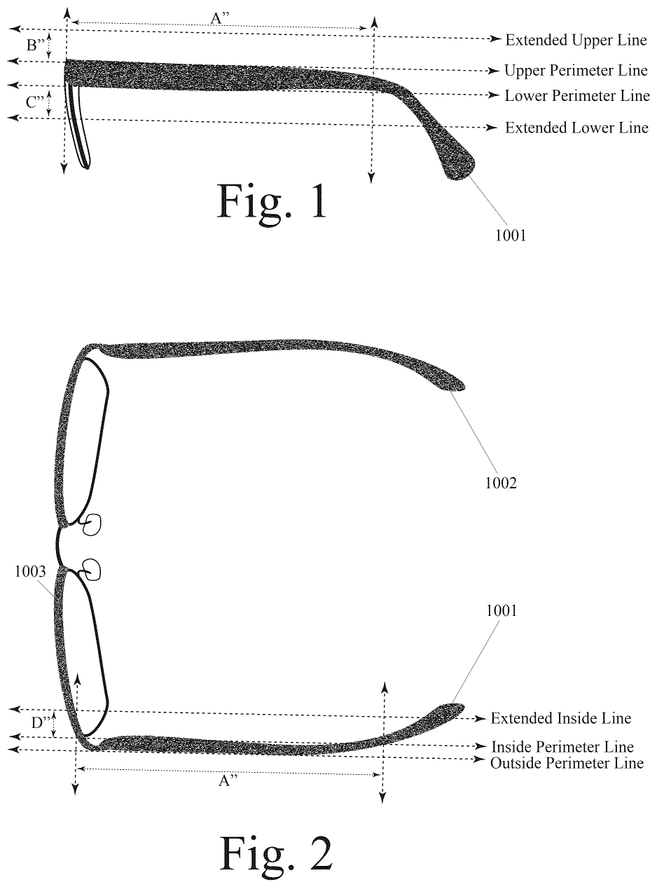

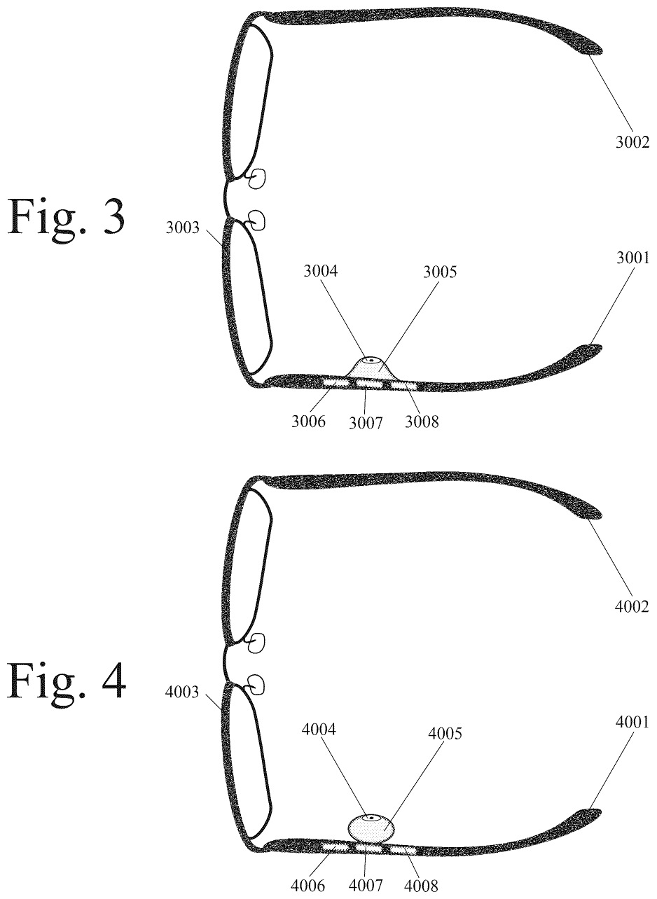

FIGS. 3 through 48 show examples of how this invention can be embodied in “EEG Glasses” (electroencephalographic eyewear). Before showing these examples, FIGS. 1 and 2 are provided to define virtual lines which can be used to specify embodiments of this invention more precisely. FIGS. 1 and 2 show side and top-down views, respectively, of a frame for eyeglasses or other eyewear, wherein this frame includes: first side section 1001; second side section 1002; and front section 1003. In an example, a first side section can be a left side section and a second side section can be a right side section, or vice versa.

FIGS. 1 and 2 show how virtual perimeter lines and extended lines can be defined for one side section of this frame for eyeglasses or other eyewear. In this example, virtual perimeter lines and extended lines are defined for side section 1001. These perimeter lines and extended lines are used subsequently in this disclosure in order to more-precisely specify the locations and dimensions of various embodiments of this invention. FIGS. 1 and 2 show how the following seven virtual straight lines can be defined for a side section of the frame for eyeglasses or other eyewear. These seven virtual straight lines are: Upper Perimeter Line; Extended Upper Line; Lower Perimeter Line; Extended Lower Line; Inside Perimeter Line; Extended Inside Line; and Outside Perimeter Line. The relative locations of these lines are specified using inch-based distance parameters A″, B″, C″ and D″. It is to be understood that metric equivalents can be substituted for inch-based measurements.

As shown in FIG. 1, the Upper Perimeter Line is the virtual straight line which most closely fits the upper perimeter of the anterior (front) A″ of the side section of a frame for eyeglasses or other eyewear as viewed from a side perspective. Closeness of fit can be determined by minimizing the sum of squared deviations (distances) from the virtual straight line to the upper perimeter of the side section. As shown in FIG. 1, the Extended Upper Line is a virtual straight line which is parallel to the Upper Perimeter Line and B″ (directly) above the Upper Perimeter Line. As shown in FIG. 1, the Lower Perimeter Line is the virtual straight line which is parallel to the Upper Perimeter Line and most closely fits the lower perimeter of the anterior (front) A″ of the side section as viewed from a side perspective. Closeness of fit can be determined by minimizing the sum of squared deviations from the virtual straight line to the lower perimeter of the side section. As shown in FIG. 1, the Extended Lower Line is the virtual straight line which is parallel to the Lower Perimeter Line and C″ (directly) below the Lower Perimeter Line. In an example, the Upper Perimeter Line, Extended Upper Line, Lower Perimeter Line, and Extended Lower Line can all be in the same vertical plane.

As shown in FIG. 2, the Inside Perimeter Line is the virtual straight line which most closely fits the inside (configured to face toward the person's head when worn) perimeter of the anterior (front) A″ of the side section of a frame for eyeglasses or other eyewear as viewed from a top-down perspective. Closeness of fit can be determined by minimizing the sum of squared deviations (distances) from a virtual straight line to the inside perimeter of the side section. As shown in FIG. 2, the Extended Inside Line is the virtual straight line which is parallel to the Inside Perimeter Line and D″ (directly) toward the person's head from the Inside Perimeter Line. As shown in FIG. 2, the Outside Perimeter Line is the virtual straight line which is parallel to the Upper Perimeter Line and most closely fits the outside perimeter of the anterior (front) A″ of the side section as viewed from a top-down perspective. Closeness of fit can be determined by minimizing the sum of squared deviations from the virtual straight line to the outside perimeter of the side section. In an example, the Inside Perimeter Line, Extended Inside Line, and Outside Perimeter Line can all be in the same horizontal plane.

Alternatively, these virtual lines can be defined based on the front half of the side section rather than the front A″ of the side section. For example: an Upper Perimeter Line can be defined as the virtual straight line which most closely fits the upper perimeter of the anterior (front) half of the side section of a frame for eyeglasses or other eyewear as viewed from a side perspective, the Lower Perimeter Line can be defined as the virtual straight line which is parallel to the Upper Perimeter Line and most closely fits the lower perimeter of the anterior (front) half of the side section as viewed from a side perspective; the Inside Perimeter Line can be defined as the virtual straight line which most closely fits the inside (configured to face toward the person's head when worn) perimeter of the anterior (front) half of the side section the side section of a frame for eyeglasses or other eyewear as viewed from a top-down perspective; and the Outside Perimeter Line can be defined as the virtual straight line which is parallel to the Upper Perimeter Line and most closely fits the outside perimeter of the anterior (front) half the side section as viewed from a top-down perspective.

In an example, a three-dimensional space can be defined as the space with an upper vertical boundary at the height of the Extended Upper Line, with a lower vertical boundary at the height of the Extended Lower Line, with an inner horizontal boundary at the horizontal location of the Extended Inside Line, and an outer horizontal boundary at the horizontal location of the Outside Perimeter Line. In an example, this three-dimensional space can be a longitudinal shape with (the same shape or varying shape) quadrilateral cross-sections. In an example, a flexible protrusion which is configured to hold an electromagnetic energy sensor on a person's head can be contained within this virtual three-dimensional space. In an example, a flexible protrusion which is attached to a side section of a frame of eyeglasses or other eyewear and which is configured to hold an electromagnetic energy sensor on a person's head can be contained within this virtual three-dimensional space related to this side section.

In an example, A″ can be a number of inches selected from within the range of 2″ and 6″. In an example, A″ can be a number of inches selected from within the range of 3″ and 5″. In an example, A″ can equal 3″. In an example, A″ can equal 4″. In an example, B″ can be a number of inches selected from within the range of 0″ and 3″. In an example, B″ can be a number of inches selected from within the range of 0″ and 2″. In an example, B″ can equal 0″. In an example, B″ can equal ½″. In an example, B″ can equal 1″. In an example, C″ can be a number of inches selected from within the range of 0″ and 2″. In an example, C″ can be a number of inches selected from within the range of 0″ and 1″. In an example, C″ can equal 0″. In an example, C″ can equal ½″. In an example, D″ can be a number of inches selected from within the range of 0″ and 2″. In an example, D″ can be a number of inches selected from within the range of 0″ and 1″. In an example, D″ can equal ½″. In an example, D″ can equal 1″. In an example, A″=4″, B″=½″, C″=½″, and D″=1″. In an example, A″=4″, B″=0″, C″=0″, and D″=1″. It is to be understood that metric equivalents can be substituted for inch-based measurements.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; wherein the flexible protrusion is contained within a three-dimensional space with an upper vertical boundary at the height of an Extended Upper Line, a lower vertical boundary at the height of an Extended Lower Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the anterior A″ of the selected side section; wherein the Extended Upper Line is a virtual straight line which is parallel to the Upper Perimeter Line and B″ above the Upper Perimeter Line; wherein a Lower Perimeter Line is the virtual straight line which is parallel to the Upper Perimeter Line and most closely fits the lower perimeter of the anterior A″ of the selected side section; wherein the Extended Lower Line is the virtual straight line which is parallel to the Upper Perimeter Line and C″ below the Lower Perimeter Line; wherein an Inside Perimeter Line is the virtual straight line which most closely fits the inside perimeter of the anterior A″ of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the anterior A″ of the selected side section; and wherein A″ is 6″ or less, B″ is ½″ or less, C″ is ½″ or less, and D″ is 2″ or less; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; wherein the flexible protrusion is contained within a three-dimensional space with an upper vertical boundary at the height of an Extended Upper Line, a lower vertical boundary at the height of an Extended Lower Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the selected side section; wherein the Extended Upper Line is a virtual straight line which is parallel to the Upper Perimeter Line and B″ above the Upper Perimeter Line; wherein a Lower Perimeter Line is the virtual straight line which is parallel to the Upper Perimeter Line and most closely fits the lower perimeter the selected side section; wherein the Extended Lower Line is the virtual straight line which is parallel to the Upper Perimeter Line and C″ below the Lower Perimeter Line; wherein an Inside Perimeter Line is the virtual straight line which most closely fits the inside perimeter of the anterior A″ of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the selected side section; and B″ is ½″ or less, C″ is ½″ or less, and D″ is 2″ or less; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; wherein the flexible protrusion is contained within a three-dimensional space with an upper vertical boundary at the height of an Upper Perimeter Line, a lower vertical boundary at the height of a Lower Perimeter Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the selected side section; wherein a Lower Perimeter Line is the virtual straight line which is parallel to the Upper Perimeter Line and most closely fits the lower perimeter the selected side section; wherein an Inside Perimeter Line is the virtual straight line which most closely fits the inside perimeter of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the selected side section; and D″ is 2″ or less; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; and wherein this flexible protrusion is configured to be located between the person's head and the selected side section; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section and which protrudes from the selected side section toward the person's head; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section and is not visible from an outer side view of the frame; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible protrusion which is part of, or attached to, a selected side section, wherein the upper perimeter of the flexible protrusion is not more than ¼″ higher than the upper perimeter of the selected side section, and wherein the lower perimeter of the flexible protrusion is not more than ¼″ lower than the lower perimeter of the selected side section; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section; wherein the selected side section is selected from the group consisting of the first section and the second section; wherein the flexible and/or compressible protrusion is contained within a three-dimensional space with an upper vertical boundary at the height of an Extended Upper Line, a lower vertical boundary at the height of an Extended Lower Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the anterior A″ of the selected side section; wherein the Extended Upper Line is a virtual straight line which is parallel to the Upper Perimeter Line and B″ above the Upper Perimeter Line; wherein a Lower Perimeter Line is the virtual straight line which is parallel to the Upper Perimeter Line and most closely fits the lower perimeter of the anterior A″ of the selected side section; wherein the Extended Lower Line is the virtual straight line which is parallel to the Upper Perimeter Line and C″ below the Lower Perimeter Line; wherein an Inside Perimeter Line is the virtual straight line which most closely fits the inside perimeter of the anterior A″ of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the anterior A″ of the selected side section; and wherein A″ is 6″ or less, B″ is ½″ or less, C″ is ½″ or less, and D″ is 2″ or less; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section; wherein the selected side section is selected from the group consisting of the first section and the second section; wherein the flexible and/or compressible protrusion is contained within a three-dimensional space with an upper vertical boundary at the height of an Extended Upper Line, a lower vertical boundary at the height of an Extended Lower Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the selected side section; wherein the Extended Upper Line is a virtual straight line which is parallel to the Upper Perimeter Line and B″ above the Upper Perimeter Line; wherein a Lower Perimeter Line is the virtual straight line which is parallel to the Upper Perimeter Line and most closely fits the lower perimeter the selected side section; wherein the Extended Lower Line is the virtual straight line which is parallel to the Upper Perimeter Line and C″ below the Lower Perimeter Line; wherein an Inside Perimeter Line is the virtual straight line which most closely fits the inside perimeter of the anterior A″ of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the selected side section; and B″ is ½″ or less, C″ is ½″ or less, and D″ is 2″ or less; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section; wherein the selected side section is selected from the group consisting of the first section and the second section; wherein the flexible and/or compressible protrusion is contained within a three-dimensional space with an upper vertical boundary at the height of an Upper Perimeter Line, a lower vertical boundary at the height of a Lower Perimeter Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the selected side section; wherein a Lower Perimeter Line is the virtual straight line which is parallel to the Upper Perimeter Line and most closely fits the lower perimeter the selected side section; wherein an Inside Perimeter Line is the virtual straight line which most closely fits the inside perimeter of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the selected side section; and D″ is 2″ or less; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section; wherein the selected side section is selected from the group consisting of the first section and the second section; and wherein this flexible and/or compressible protrusion is configured to be located between the person's head and the selected side section; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section and which protrudes from the selected side section toward the person's head; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section and is not visible from an outer side view of the frame; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, this invention can comprise: (a) a frame for eyeglasses or other eyewear; wherein this frame further comprises a front section which is configured to span the front of a person's head, a first side section which is configured to span from a first ear to the front section, and a second side section which is configured to span from a second ear to the front section; (b) a flexible and/or compressible protrusion which is part of, or attached to, a selected side section, wherein the upper perimeter of the flexible and/or compressible protrusion is not more than ¼″ higher than the upper perimeter of the selected side section, and wherein the lower perimeter of the flexible and/or compressible protrusion is not more than ¼″ lower than the lower perimeter of the selected side section; (c) an electromagnetic energy sensor which collects data concerning electromagnetic brain activity; wherein the flexible and/or compressible protrusion is configured to hold the electromagnetic energy sensor in proximity to and/or against the person's head; (d) an energy source; (e) a data processor; and (f) a data transmitter and/or receiver.

In an example, a frame for eyeglasses or other eyewear can be part of a device selected from the group consisting of: Augmented Reality (AR) glasses or other AR eyewear, electronically-functional eyeglasses, electronically-functional eyewear, electronically-functional goggles, electronically-functional visor, eyeglasses with integrated camera, eyewear-based human-to-computer interface, goggles, mobile EEG monitoring eyewear, non-prescription eyeglasses, prescription eyeglasses, smart eyewear, smart glasses, smart sunglasses, and Virtual Reality (VR) glasses or other VR eyewear. In an example, this invention can comprise eyewear which is selected from the group consisting of: Augmented Reality (AR) glasses or other AR eyewear, electronically-functional eyeglasses, electronically-functional eyewear, electronically-functional goggles, electronically-functional visor, eyeglasses with integrated camera, eyewear-based human-to-computer interface, goggles, mobile EEG monitoring eyewear, non-prescription eyeglasses, prescription eyeglasses, smart eyewear, smart glasses, smart sunglasses, and Virtual Reality (VR) glasses or other VR eyewear.

In an example, a front section of a frame (for glasses or other eyewear) can span the front of a person's head in the space in front of a person's eyes and/or eyebrows. In an example, a frame can hold one or more optical elements. In an example, a frame can hold one or more optical lenses. In an example, a frame can hold one or more computer displays. In an example, a frame can hold one or more optical lenses and one or more computer displays. In an example, an optical element can function as both a lens and a computer display.

In an example, the front section, first side section, and second side section (of a frame for eyeglasses or other eyewear) can each be substantially straight. In an example, the front section, first side section, and second side section of a frame can each be curved and arcuate. In an example, a frame for eyeglasses or other eyewear can be configured like conventional eyeglasses except for an upward wave or bulge in one or both side sections. In an example, a frame for eyeglasses or other eyewear can be configured like conventional eyeglasses except for an upward sinusoidal wave or bulge in one or both side sections.

In an example, a side section of a frame for glasses or other eyewear can be curved, arcuate, undulating, wavy, and/or sinusoidal. In an example, a side section can have a single upward (sinusoidal) wave within its anterior (front) half and an electromagnetic energy sensor located on (the upper portion of) this (sinusoidal) wave. In an example, a side section can have a multiple (sinusoidal) waves along its longitudinal (front to rear) axis. In an example, a side section can bifurcate into upper and lower side portions and have an electromagnetic energy sensor located on the upper portion. In an example, a side section can include a circular, oval, or elliptical portion within its anterior (front) half and an electromagnetic energy sensor located on the upper part of this portion.

In an example, a side section of a frame for glasses or other eyewear can be configured to: start with a posterior end behind a person's ear; then curve upward and forward around portion of the ear which connects to the main body of the head; then extend forward 1″-2″ in a relatively straight manner; then curve upward (in a sinusoidal manner) to span a portion of the person's temple and/or forehead; and then curve back downward (in a sinusoidal manner) to connect to the front section. In an example, a side section of a frame for glasses or other eyewear can be configured to: start with a posterior end behind a person's ear; then curve upward and forward around portion of the ear which connects to the main body of the head; then curve downward (in a sinusoidal manner) in front of the person's ear; then curve upward (in a sinusoidal manner) to span a portion of the person's temple and/or forehead; and then curve back downward (in a sinusoidal manner) to connect to the front section.

In an example a side section of a frame (for glasses or other eyewear) can have an average vertical width in the range of 1/16″ to 2″. In an example, a side section of a frame can have a vertical width which varies within the range of 1/16″ to 2″. In an example a side section of a frame (for glasses or other eyewear) can have an average vertical width in the range of ¼″ to 1″. In an example, a side section of a frame can have a vertical width which varies within the range of ¼″ to 1″. It is to be understood that metric equivalents can be substituted for inch measurements throughout this disclosure.

In example, a side section of a frame can have a central posterior-to-anterior (back-to-front) longitudinal axis. In an example, a posterior (e.g. rear one-third) portion of this posterior-to-anterior longitudinal axis can curve around the rear of a person's ear and an anterior (e.g. front, two-thirds) portion of this longitudinal axis can span from the ear to the front section of the frame in a relatively straight manner. In an example, a posterior (e.g. rear one-third) portion of this posterior-to-anterior longitudinal axis can curve around the rear of a person's ear and an anterior (e.g. front, two-thirds) portion of this longitudinal axis can span from the ear to the front section of the frame in an arcuate (e.g. sinusoidal) manner which spans a portion of a person's temple and/or forehead. In an example, a posterior (one-third) portion of this posterior-to-anterior longitudinal axis can curve around the rear of a person's ear, a middle (one-third) portion of this longitudinal axis can span from the ear toward the front in a relatively straight manner, and an anterior (one-third) portion of this longitudinal axis can span a portion of a person's temple and/or forehead in an arcuate (sinusoidal or conic section) manner.

In an example, the front section, first side section, and second side section of a frame can be portions of a single continuous piece of material. In an example, the front section, first side section, and second side section of a frame can be separate pieces of material which are connected to each other by hinges, joints, or welds. In an example, a frame for eyeglasses or other eyewear can be made of metal, plastic, fabric, or a combination thereof. In an example, the front section, first side section, and second side section of a frame can each be rigid or semi-rigid. In an example, the front section, first side section, and second side section of a frame can each be flexible and/or elastic. In an example, one or more sections selected from the group consisting of the front section, first side section, and second side section of a frame can be rigid and one or more sections selected from this group can be flexible and/or elastic.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; and (c) then span forward in a relatively-straight axial manner to an anterior (front) end which connects to (or becomes part of) a front section of the frame. In an example, the portion of the side section described in (c) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; and (c) then span forward 3″-5″ in a relatively-straight axial manner to an anterior (front) end which connects to (or becomes part of) a front section of the frame. In an example, the portion of the side section described in (c) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then span forward in a relatively-straight axial manner; (d) then curve upward and forward to a location over the person's temple and/or forehead; and (e) then curve downward and forward to an anterior (front) end which connects to (or becomes part of) a front section of the frame. In an example, a portion of the side section described in (d) or (e) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then span forward 1″-3″ in a relatively-straight axial manner; (d) then curve upward and forward 1″-3″ to a location over the person's temple and/or forehead; and (e) then curve downward and forward to an anterior (front) end which connects to (or becomes part of) a front section of the frame. In an example, a portion of the side section described in (d) or (e) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then curve downward and forward; (d) then curve upward and forward to a location over the person's temple and/or forehead; and (e) then curve downward and forward to an anterior (front) end which connects to (or becomes part of) a front section of the frame. In an example, a portion of the side section described in (d) or (e) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then curve downward and forward 1″-3″; (d) then curve upward and forward 1″-3″ to a location over the person's temple and/or forehead; and (e) then curve downward and forward to an anterior (front) end which connects to (or becomes part of) a front section of the frame. In an example, a portion of the side section described in (d) or (e) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then curve downward and forward; (d) then curve upward, forward, and inward to a location over the person's temple and/or forehead; and (e) then curve downward, forward, and outward to an anterior (front) end which connects to (or becomes part of) a front section of the frame. In an example, a portion of the side section described in (d) or (e) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then curve downward and forward 1″-3″; (d) then curve upward, forward, and inward 1″-3″ to a location over the person's temple and/or forehead; and (e) then curve downward, forward, and outward to an anterior (front) end which connects to (or becomes part of) a front section of the frame. In an example, a portion of the side section described in (d) or (e) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then curve downward and forward; (d) then curve upward and forward to bifurcate, wherein an upper portion of this bifurcation extends forward over the person's temple and/or forehead, and wherein the lower portion of this bifurcation curves forward to connect to a front section of the frame. In an example, the upper portion of a bifurcation described in (d) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then bifurcate, wherein an upper portion of this bifurcation extends to the person's temple and/or forehead, and wherein a lower portion of this bifurcation spans forward in a relatively-straight axial manner to an anterior (front) end which connects to (or becomes part of) a front section of the frame. In an example, the upper and lower portion can connect to each other at both posterior and anterior locations. In an example, the upper portion of a bifurcation described in (c) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then bifurcate, wherein an upper portion of this bifurcation extends 0.5″ to 4″ to the person's temple and/or forehead, and wherein a lower portion of this bifurcation spans forward in a relatively-straight axial manner to an anterior (front) end which connects to (or becomes part of) a front section of the frame. In an example, the upper and lower portion can connect to each other at both posterior and anterior locations. In an example, the upper portion of a bifurcation described in (c) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then bifurcate, wherein an upper portion of this bifurcation curves around the person's temple and/or forehead, and wherein a lower portion of this bifurcation spans forward in a relatively-straight axial manner to an anterior (front) end which connects to (or becomes part of) a front section of the frame. In an example, upper and lower portions can be connected to each other at two or more locations, with a gap between the portions between these connections. In an example, the upper portion of a bifurcation described in (c) can hold an electromagnetic energy sensor on a person's head.

In an example, a side section of a frame for eyeglasses or other eyewear can be configured to span forward from the rear portion of a person's ear in the following manner: (a) start with a posterior (rear) end which is configured to be worn posterior to (behind) a person's ear; (b) then curve upward and forward around the tissue connection between the person's outer ear to the rest of the person's head, to the top of this tissue connection; (c) then expand, fan out, broaden, and/or widen into a fin or wedge shaped structure which curves around a side portion of the person's forehead; and then connects to a front section of the frame. In an example, the portion of the side section described in (c) can hold an electromagnetic energy sensor on a person's head.

In an example, a sensor-holding protrusion which is part of, or attached to, a side section of a frame can be flexible. In an example, a protrusion can be a flexible piece of metal, plastic, or fabric. In an example, a protrusion can be compressible. In an example, a protrusion can be a compressible foam component. In an example, a protrusion can be inflatable. In an example, a protrusion can be a balloon or other type of inflatable compartment. In an example, a protrusion can be pleated and/or folded (like a bellows) for expansion or contraction. In an example, a protrusion can be elastic. In an example, a protrusion can be an elastic band or strip. In an example, a protrusion can be tensile. In an example, a protrusion can be a spring, coil, or other tensile member. In an example, a protrusion can be a piston or other telescoping structure.

In an example, a protrusion which holds an electromagnetic energy sensor can have a shape selected from the group consisting of: arcuate, bell curve shaped, bellows, circular, conic, conic-section shaped, cylindrical, egg-shaped, elliptical, frustal, half sinusoidal, half-bell curve shaped, helical, kidney-bean shaped, oval, parabolic, piston, pyramidic, quarter sinusoidal, rounded rectangular, rounded square, sinusoidal, spherical, S-shapes, telescoping, triangular, and wedge shaped. In an example, a protrusion can be transparent or translucent so as to be less obvious.

In an example, a protrusion can be contained within a vertical space that is upward bounded by the height of an Upper Perimeter Line and downward bounded by the height of a Lower Perimeter Line. In this case, the protrusion should not be visible from an outer side view perspective of the eyewear side frame. In an example, a protrusion can be contained within a vertical space that is upward bounded by the height of an Extended Upper Line and downward bounded by the height of an Extended Perimeter Line. In an example, the Extended Upper Line can be up to 1″ (or the metric equivalent) above the Upper Perimeter Line and the Extended Lower Line can be up to 1″ (or the metric equivalent) below the Lower Perimeter Line.

In an example, only one side section of a frame for eyeglasses or other eyewear can have a protrusion which holds an electromagnetic energy sensor. In an example, eyewear can have a unilateral electromagnetic energy sensor. In an example, each of the two side sections of a frame for eyeglasses or other eyewear can have a protrusion which holds an electromagnetic energy sensor. In an example, eyewear can have bilateral electromagnetic energy sensors. In an example, one or both side sections can have multiple protrusions and/or multiple electromagnetic energy sensors. In an example, multiple protrusions and/or multiple electromagnetic energy sensors on a side section of an eyewear frame can span a range between 1″ and 4″.

In an example, a protrusion can apply force to the outer side of an electromagnetic energy sensor (which faces away from a person's body) so that the side of the inner side of an electromagnetic energy sensor (which faces toward the person's body) exerts force on the person's body. This can achieve better electromagnetic communication with the person's body. In an example, a protrusion can gently press an electromagnetic energy sensor against a person's head. In an example, a protrusion can include a spring mechanism which gently presses an electromagnetic energy sensor against a person's head. In an example, a protrusion can include an elastic mechanism which gently presses an electromagnetic energy sensor against a person's head. In an example, a protrusion can include an inflatable mechanism (filled with a gas or liquid) which gently presses an electromagnet energy sensor against a person's head.

In an example, the location of a protrusion (and thus an electromagnetic energy sensor which it holds) can be manually or automatically moved with respect to the side section of an eyewear frame. In an example, the location of a protrusion (and associated electromagnetic energy sensor) can be moved forward or backward, such as along a track or channel on the side section of an eyewear frame. In an example, the location of a protrusion (and associated electromagnetic energy sensor) can be moved up or down, such as along a track or channel on the side section of an eyewear frame. In an example, the angle of a protrusion with respect to the side section of an eyewear frame can be manually or automatically adjusted. In an example, a protrusion can be attached to different locations along the side section of an eyewear frame.

In an example, the location of contact between an electromagnetic energy sensor and a person's head can be adjusted by adjusting the location of a protrusion by one or more actions selected from the following group: sliding the protrusion along a track or channel on a side section of an eyewear frame; clipping the protrusion to different locations along the side section of an eyewear frame, rotating a threaded protrusion; adjusting the inflation pressure of an inflated protrusion; pneumatic adjustment of a liquid-filled protrusion; adjustment of a piston or other telescoping structure, adjusting the spring and/or coil tension of a protrusion comprising a spring and/or coil; and adjusting the magnetic attraction or repulsion of a magnetic protrusion.

In an example, the force and/or pressure of contact between an electromagnetic energy sensor and a person's head can be adjusted by adjusting the location of a protrusion by one of more actions selected from the following group: sliding the protrusion along a track or channel on a side section of an eyewear frame; clipping the protrusion to different locations along the side section of an eyewear frame, rotating a threaded protrusion; adjusting the inflation pressure of an inflated protrusion; pneumatic adjustment of a liquid-filled protrusion; adjusting the spring and/or coil tension of a protrusion comprising a spring and/or coil; and adjusting the magnetic attraction or repulsion of a magnetic protrusion.

In an example, this invention can further comprise one or more force and/or pressure sensors which measure the force and/or pressure applied by an electromagnetic energy sensor against a person's head (e.g. temple and/or forehead). In an example, this invention can adjust the configuration and/or location of a flexible protrusion so as to adjust the force and/or pressure applied by an electromagnetic energy sensor against a person's head (e.g. temple and/or forehead).

If data from a force and/or pressure sensor indicates inadequate force and/or pressure applied by an electromagnetic energy sensor, then this can trigger adjustment of the configuration and/or location of a flexible protrusion which holds the electromagnetic energy sensor so as to increase the force and/or pressure applied by an electromagnetic energy sensor. If data from a force and/or pressure sensor indicates excessive force and/or pressure applied by an electromagnetic energy sensor, then this can trigger adjustment of the configuration and/or location of a flexible protrusion which holds the electromagnetic energy sensor so as to decrease the force and/or pressure applied by an electromagnetic energy sensor.

In an example, if a force and/or pressure sensor indicates inadequate force and/or pressure applied by an electromagnetic energy sensor to a person's forehead, then this can trigger increased inflation of a flexible protrusion which holds the electromagnetic energy sensor. In an example, if a force and/or pressure sensor indicates excessive force and/or pressure applied by an electromagnetic energy sensor to a person's forehead, then this can trigger decreased inflation of a flexible protrusion which holds the electromagnetic energy sensor. In an example, if a force and/or pressure sensor indicates inadequate force and/or pressure applied by an electromagnetic energy sensor to a person's forehead, then this can trigger (pneumatic) extension of a telescoping protrusion which holds the electromagnetic energy sensor. In an example, if a force and/or pressure sensor indicates excessive force and/or pressure applied by an electromagnetic energy sensor to a person's forehead, then this can trigger (pneumatic) retraction of a telescoping protrusion which holds the electromagnetic energy sensor.

In an example, if a force and/or pressure sensor indicates inadequate force and/or pressure applied by an electromagnetic energy sensor to a person's forehead, then this can trigger threaded rotation and extension of a protrusion which holds the electromagnetic energy sensor. In an example, if a force and/or pressure sensor indicates excessive force and/or pressure applied by an electromagnetic energy sensor to a person's forehead, then this can trigger threaded rotation and retraction of a protrusion which holds the electromagnetic energy sensor. In an example, if a force and/or pressure sensor indicates inadequate force and/or pressure applied by an electromagnetic energy sensor to a person's forehead, then this can trigger electromagnetic actuator extension of a protrusion which holds the electromagnetic energy sensor. In an example, if a force and/or pressure sensor indicates excessive force and/or pressure applied by an electromagnetic energy sensor to a person's forehead, then this can trigger electromagnetic actuator retraction of a protrusion which holds the electromagnetic energy sensor.

In an example, an electromagnetic energy sensor of this invention can measure the conductivity, voltage, resistance, impedance, and/or permittivity of electromagnetic energy transmitted through and/or emitted from a portion of a person's brain and/or head. In an example, an electromagnetic energy sensor can be an electroencephalographic (EEG) sensor. In an example, an electromagnetic energy sensor can be a dry electrode. In an example, an electromagnetic energy sensor can collect data on electromagnetic energy patterns and/or electromagnetic fields which are naturally generated by electromagnetic brain activity. In an example, an electromagnetic energy sensor can be used in combination with an electromagnetic energy emitter. In an example, an electromagnetic energy emitter can be in contact with the surface of a person's head. In an example, an electromagnetic energy sensor can measure the conductivity, voltage, resistance, impedance, and/or permittivity of electromagnetic energy emitted from an electromagnetic energy emitter and transmitted through a portion of a person's head.

In an example, the location of an electromagnetic energy sensor can be selected from the group of standard electrode locations consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, data from one or more electromagnetic energy sensors can be filtered to remove artifacts before the application of a primary statistical method. In an example, a filter can be used to remove electromagnetic signals from eye blinks, eye flutters, or other eye movements before the application of a primary statistical method. In an example, a notch filter can be used as well to remove 60 Hz artifacts caused by AC electrical current. In various examples, one or more filters can be selected from the group consisting of: a high-pass filter, a band-pass filter, a loss-pass filter, an electromyographic activity filter, a 0.5-1 Hz filter, and a 35-70 Hz filter.

In an example, data from an electromagnetic energy sensor can be analyzed using Fourier transformation methods in order to identify repeating energy patterns in clinical frequency bands. In an example, these clinical frequency bands can be selected from the group consisting of: Delta, Theta, Alpha, Beta, and Gamma. In an example, the relative and combinatorial power levels of energy in two or more different clinical frequency bands can be analyzed. In an example, a person can receive real-time feedback based on analysis of data concerning their electromagnetic brain activity. In an example, a person can control a computer or other device by self-modifying their electromagnetic brain activity.

In an example, an energy source for this device can be a battery internal to the device. In an example, an energy source can be internal to the device during regular operation (such as an internal battery, capacitor, energy-storing microchip, or wound coil or spring). In an example, an energy source can harvest and/or transduce energy from a person's body (such as kinetic or mechanical energy from body motion, electromagnetic energy from the person's body, blood flow or other internal fluid flow, glucose metabolism, or thermal energy from the person's body). In an example, an energy source can harvest and/or transduce energy from a source external to the device (such as electromagnetic inductance from external source, solar energy, indoor lighting energy, wired connection to an external power source, ambient or localized radiofrequency energy, or ambient thermal energy). In an example, an energy source can be selected from the group consisting of: a rechargeable or replaceable battery; an energy harvesting member which harvests, transduces, or generates energy from body motion or kinetic energy, body thermal energy, or body biochemical energy; an energy harvesting member which harvests, transduces, or generates energy from ambient light energy or ambient electromagnetic energy.

In an example, a data processor can be a computer. In an example, a data processor can be a central processing unit (CPU). In an example, a data processor can be a computer chip or board. In an example, a data processor can be in electromagnetic communication with an energy source, one or more electromagnetic energy sensors, and a data transmitter/receiver via wires or other electrically-conductive pathways. In an example, a data processor can process data from one or more electromagnetic energy sensors to analyze data patterns. In an example, a data processor can receive data from one or more electromagnetic energy sensors and relay this data to a data transmitter/receiver which, in turn, sends this data to a separate (remote) data processor which analyzes data patterns.

In an example, a data processor can be in wireless communication with a separate wearable device selected from the group consisting of: a wristwatch, smart watch, fitness watch, watch phone, bracelet phone, smart bracelet, fitness bracelet, smart wrist band, electronically-functional wrist band, other wrist-worn electronic device, or smart armband; a smart button, electronically-functional button, pin, brooch, pendant, beads, neck chain, necklace, dog tags, locket, or medallion; a smart finger ring, electronically-functional finger ring, electronically-functional earring, nose ring, or ear bud or clip; a wearable camera; an article of smart clothing, an electronically-functional shirt, electronically-functional pants, or a smart belt.

In an example, a data processor can be in wireless communication with a separate mobile device selected from the group consisting of: smart phone, mobile phone, holophone, or cellular phone; PDA; electronic tablet; electronic pad; and other electronically-functional handheld device. In an example, a data processor can be in wireless communication with a relatively fixed-location device selected from the group consisting of: laptop computer, desktop computer, internet terminal, smart appliance, home control system, and other fixed-location electronic communication device.

In an example, this device can further comprise a human-to-computer interface selected from the group consisting of: a button, knob, or dial; a display screen; a gesture-recognition interface; a holographic user interface; a microphone; a physical keypad or keyboard; a pressure-sensitive textile array; a spectroscopic sensor; a speech or voice recognition interface; a touch screen; a virtual keypad or keyboard; an electronically-functional textile interface; and an eye gaze tracker.

In an example, this device can further comprise a computer-to-human interface selected from the group consisting of: a coherent-light image projector; a display screen; a holographic user interface; a laser; a myostimulating member; a neurostimulating member; a non-coherent-light image projector; a speaker or other sound-emitting member; a speech or voice recognition interface; a synthesized voice; a vibrating or other tactile sensation creating member; an electromagnetic energy emitter; an electronically-functional textile interface; an infrared light emitter; an infrared light projector; and an LED or LED array.

In an example, this device an further comprise one or more motion-related sensors selected from the group consisting of: dual-axial accelerometer, tri-axial accelerometer, other multi-axial accelerometer, gyroscope, inclinometer or tilt sensor, goniometer, GPS or other location sensor, other inertial or motion sensor, goniometer, and kinematic sensor. In an example, this invention can further comprise one or more other types of electromagnetic energy sensors selected from the group consisting of: peripheral neurosensor, electromyography (EMG) sensor, Hall-effect sensor, electrocardiogram (ECG) sensor, cardiac monitor, EOG sensor, galvanic skin response (GSR) sensor, compass, magnometer, magnetic sensor, potentiometer, variable-resistance sensor, resistive bend sensor, piezoelectric sensor, piezomechanical sensor, and piezoresistive sensor.

In an example, this device can further comprise one or more optical sensors selected from the group consisting of: camera, other imaging member, photoelectric sensor, light intensity sensor, infrared light sensor, ultraviolet light sensor, spectroscopy sensor, near-infrared spectroscopy sensor, Raman spectroscopy sensor, spectral analysis sensor, spectrometry sensor, spectrophotometer sensor, chromatography sensor, other light-spectrum-analyzing sensor, fluorescence sensor, blood oximetry sensor, optoelectronic sensor, optical code scanner, laser sensor, optical strain detector, and variable-translucence sensor.

In an example, this device can further comprise one or more sonic energy sensors selected from the group consisting of: microphone, ultrasonic sensor, acoustic sensor, heart rate sensor, respiration or pulmonary function monitor, respiratory rate sensor, and CPAP monitor. In an example, this device can further comprise one or more biochemical sensors selected from the group consisting of: electrochemical sensor, biochemical sensor, glucose sensor, chemoreceptor sensor, gas sensor, microbial sensor, micro-sampling tissue or body fluid sensor, pH level sensor, and photochemical sensor.

In an example, this device can further comprise one or more force-related sensors selected from the group consisting of: blood pressure sensor, heart rate monitor, capacitive sensor, force sensor, particulate force transducer, electromagnetic pressure sensor, other pressure sensor, torque sensor, and torsion sensor. In an example, this device can further comprise one or more actuators selected from the group consisting of: brushless DC motor, brush-type DC motor, electric motor, electromagnetic actuator, hydraulic actuator, induction motor, MEMS actuator, piezoelectric actuator, pneumatic actuator, and stepper motor.

In an example, this device can further comprise one or more additional sensors selected from the group consisting of: humidity sensor, moisture sensor, thermometer, temperature sensor, flow sensor, differential transducer sensor, elastomeric sensor, vibration sensor, helical sensor, revolute joint sensor, ionizing radiation sensor, neurosensor, food consumption sensor, eye-tracking sensor, Micro-Electro-Mechanical System (MEMS) sensor, nanoscale sensor, nanotube sensor, and nanoparticle sensor.

In various examples, this device can comprise one or more additional wearable sensors can be selected from the group consisting of: dual-axial accelerometer, tri-axial accelerometer, other multi-axial accelerometer, gyroscope, inclinometer or tilt sensor, goniometer, GPS or other location sensor, other inertial or motion sensor, goniometer, kinematic sensor; peripheral neurosensor, electromyography (EMG) sensor, Hall-effect sensor, electrocardiogram (ECG) sensor, cardiac monitor, EOG sensor, galvanic skin response (GSR) sensor, impedance sensor, compass, magnometer, magnetic sensor, potentiometer, variable-resistance sensor, resistive bend sensor, piezoelectric sensor, piezomechanical sensor, or piezoresistive sensor; camera, other imaging member, photoelectric sensor, light intensity sensor, infrared light sensor, ultraviolet light sensor, spectroscopy sensor, near-infrared spectroscopy sensor, Raman spectroscopy sensor, spectral analysis sensor, spectrometry sensor, spectrophotometer sensor, chromatography sensor, other light-spectrum-analyzing sensor, fluorescence sensor, blood oximetry sensor, optoelectronic sensor, optical code scanner, laser sensor, optical strain detector, variable-translucence sensor; microphone, ultrasonic sensor, acoustic sensor, heart rate sensor, respiration or pulmonary function monitor, respiratory rate sensor, CPAP monitor; blood pressure sensor, heart rate monitor, capacitive sensor, force sensor, particulate force transducer, electromagnetic pressure sensor, other pressure sensor, torque sensor, torsion sensor; electrochemical sensor, biochemical sensor, glucose sensor, chemoreceptor sensor, gas sensor, microbial sensor, micro-sampling tissue or body fluid sensor, pH level sensor, photochemical sensor; Micro-Electro-Mechanical System (MEMS) sensor, nanoscale sensor, nanotube sensor, nanoparticle sensor; humidity sensor, moisture sensor; thermometer, temperature sensor; flow sensor; differential transducer sensor, elastomeric sensor, vibration sensor, smooch detector, helical sensor, revolute joint sensor, ionizing radiation sensor, neurosensor; food consumption sensor, and eye tracking sensor.

In an example, this device can further comprise one or more additional components selected from the group consisting of: accelerometer, computer-to-human interface, data memory, data memory component, display screen, electronic payment mechanism, gesture recognition capability, GPS component, gyroscope, heart rate monitor, human-to-computer interface, light, microphone, speaker, speech-recognition software, tactile actuator, touch screen, touch-activated button, vibrator, wireless data reception component, and wireless data transmitter.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), and wherein an upper perimeter of a sidepiece has a forward-angled bulge; and a brain activity sensor (e.g. EEG electrode) on the forward-angled bulge. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein (the longitudinal axis of) a sidepiece (e.g. one of the two sidepieces) has a vertically concave shape; and one or more brain activity sensors (e.g. EEG electrodes) on the sidepiece.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein (the longitudinal axis of) a sidepiece (e.g. one of the two sidepieces) has a vertically and horizontally sinusoidal and/or undulating shape; and one or more brain activity sensors (e.g. EEG electrodes) on the sidepiece. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein a middle section of a sidepiece (e.g. one of the two sidepieces) has an upward and forward-angled wave; and a brain activity sensor (e.g. EEG electrode) on the wave of the sidepiece.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein an anterior third of a sidepiece (e.g. one of the two sidepieces) has an upward and forward-angled wave; and a brain activity sensor (e.g. EEG electrode) on the wave of the sidepiece. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein an anterior third of a sidepiece (e.g. one of the two sidepieces) bifurcates into an upper branch and a lower branch; and a brain activity sensor (e.g. EEG electrode) on the upper branch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a bifurcation of a sidepiece into an upper branch and a lower branch on the anterior third of the sidepiece, wherein the opening or gap between the upper branch and the lower branch has an oval or elliptical shape; and a brain activity sensor (e.g. EEG electrode) on the upper branch. In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an upper vertical bulge on the anterior third of a sidepiece; and a brain activity sensor (e.g. EEG electrode) on the upper vertical bulge.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); wherein a sidepiece (e.g. one of the two sidepieces) bifurcates into an lower branch and an upper branch, wherein the lower branch is straight and spans the entire length of the sidepiece from an ear to the frontpiece, and wherein the upper branch is arcuate and spans between 50% and 80% of the length of the sidepiece; and one or more brain activity sensors (e.g. EEG electrodes) on the upper branch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a plurality of pivoting arms between the concavity and the sidepiece, wherein different pivoting arms extend out from the sidepiece at different angles; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein the concavity is located on the anterior third of the sidepiece; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch (e.g. band or strap) which is attached to a sidepiece (e.g. one of the two sidepieces) and curves upward from the sidepiece, and wherein the arch has a first configuration which extends a first distance from the sidepiece and a second configuration which extends a second distance from the sidepiece; and a brain activity sensor (e.g. EEG electrode) on the arch.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a pneumatic or hydraulic piston between the concavity and the sidepiece; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch whose ends are movably attached to a sidepiece, wherein movement of the ends of the flexible arch closer together causes the flexible arch to extend further out from the sidepiece and movement of the ends of the flexible arch farther apart causes the flexible arch to extend out less from the sidepiece; and a brain activity sensor (e.g. EEG electrode) on the flexible arch.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position FT9.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position F8. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position AF7.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an articulated (e.g. jointed) arch (e.g. band or strap) which is attached to a sidepiece (e.g. one of the two sidepieces), wherein the arch has a first configuration which extends out a first distance from the sidepiece, wherein the arch has a second configuration which extends out a second distance from the sidepiece; and a brain activity sensor (e.g. EEG electrode) on the arch.

In an example, eyewear (e.g. eyeglasses) with brain activity sensors (e.g. EEG electrodes) can comprise: eyewear (e.g. eyeglasses) which is worn by a person, wherein the eyewear further comprises two sidepieces (e.g. temples); a flexible band or strap, wherein a first end of the band or strap is attached to a first location (e.g. an anterior location) on a sidepiece and a second end of the band or strap is attached to second location (e.g. a posterior location) on the sidepiece; and a brain activity sensor (e.g. EEG electrode) on the band or strap.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; and a brain activity sensor (e.g. EEG electrode) on the arm. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an S-shaped movable arm on a sidepiece (e.g. one of the two sidepieces), wherein the arm extends out from the sidepiece toward the surface of the person's head; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein one end of the pivoting arm is movably-connected (e.g. by a hinge or joint) to a sidepiece (e.g. one of the two sidepieces) and the other end of the pivoting arm free to extend out from the sidepiece, wherein the angle between the pivoting arm and the sidepiece is adjustable; and a brain activity sensor (e.g. EEG electrode) on the pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein one end of the pivoting arm is movably-connected (e.g. by a hinge or joint) to a sidepiece (e.g. one of the two sidepieces) and the other end of the pivoting arm free to extend out from the sidepiece, wherein the horizontal angle between the pivoting arm and the sidepiece is adjustable, wherein the pivoting arm can be locked at a selected horizontal angle between the pivoting arm and the sidepiece; and a brain activity sensor (e.g. EEG electrode) on the pivoting arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm on a sidepiece, wherein the posterior end of the pivoting arm is movably connected to the sidepiece by a hinge or joint, wherein the anterior end of the pivoting arm can extend out from the sidepiece; one or more pistons between the pivoting arm and the sidepiece, wherein the resistance or length of the one or more pistons can be adjusted; and a brain activity sensor (e.g. EEG electrode) on the pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location FT9

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location F8.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location AF7 or AF3.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) on the anterior end of the arm. In an example, eyewear (e.g. eyeglasses) with brain activity sensors (e.g. EEG electrodes) can comprise: eyewear (e.g. eyeglasses) which is worn by a person; a pivoting arm which is attached at one end to a sidepiece (e.g. temple) of the eyewear; and at least one brain activity electrode on the pivoting arm, wherein the brain activity electrode further comprises a plurality of soft (e.g. Shore 00 value less than 70) electroconductive protrusions which are configured to extend out toward the surface of the person's head.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; a compressible piston component between the pivoting arm and the sidepiece; and at least one brain activity sensor (e.g. electrode) on the (frontal half of the) pivoting arm.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; a hydraulic chamber between the pivoting arm and the sidepiece; and at least one brain activity sensor (e.g. electrode) on the (frontal half of the) pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a tension arm (e.g. prong) which extends out from the sidepiece toward the surface of the person's head, wherein a posterior end of the tension arm is attached to the sidepiece and an anterior end of the tension arm extends out from the sidepiece toward the surface of the person's head, wherein the tension arm has a first configuration which extends out a first distance from the sidepiece, wherein the tension arm has a second configuration which extends out a second distance from the sidepiece, wherein the second distance is less than the first distance, and wherein the tension arm is moved from the first configuration to the second configuration by force from contact with the person's head; and a brain activity sensor (e.g. EEG electrode) on the tension arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate tensile protrusion which is part of, or attached to, a sidepiece (e.g. one of the two sidepieces); and a brain activity sensor (e.g. EEG electrode) on the protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a concave flexible band which extends out from a sidepiece (e.g. one of the two sidepieces) toward the person's head, wherein ends of the band are slideably-connected to the sidepiece at different locations, wherein the band has a first configuration in which the ends are a first distance from each other and the band extends out from the sidepiece by a second distance, wherein the band has a second configuration in which the ends are a third distance from each other and the band extends out from the sidepiece by a fourth distance, wherein the third distance is less than the first distance, and wherein the fourth distance is greater than the second distance; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a concave flexible band which extends out from a sidepiece (e.g. one of the two sidepieces) toward the person's head, wherein ends of the band are connected to the sidepiece at locations which are between 2 and 4 apart from each other, wherein one or both of the ends can slide along the sidepiece, and wherein sliding one or both of the ends along a track or channel on the sidepiece toward each other decreases the distance between the ends, which increases the distance by which the band extends out from the sidepiece toward the person's head; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch (e.g. band or strap) whose ends are slideably-attached to a sidepiece (e.g. one of the two sidepieces) and which curves upward from the sidepiece, and wherein the arch has a first configuration which extends a first distance from the sidepiece and a second configuration which extends a second distance from the sidepiece; and a brain activity sensor (e.g. EEG electrode) on the arch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible protrusion whose ends are slideably attached to a sidepiece, wherein sliding the ends of the flexible protrusion closer together causes the flexible arch to extend further out from the sidepiece and sliding the ends of the flexible protrusion farther apart causes the flexible protrusion to extend out less from the sidepiece; and a brain activity sensor (e.g. EEG electrode) on the flexible protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) protrusion (e.g. arch) on a sidepiece (e.g. one of the two sidepieces), wherein an anterior portion (e.g. an anterior end) of the protrusion, a posterior portion (e.g. an posterior end) of the protrusion, or both are movably-attached (e.g. slideably-connected) to the sidepiece, wherein the protrusion has a first configuration in which there is a first distance between the anterior portion and the posterior portion and wherein the protrusion extends out a second distance from the sidepiece, wherein the protrusion has a second configuration in which there is a third distance between the anterior portion and the posterior portion and wherein the protrusion extends out a fourth distance from the sidepiece, wherein the third distance is less than the first distance, wherein the fourth distance is greater than the second distance, and wherein the anterior portion and/or the posterior portion slide along the sidepiece; and a brain activity sensor (e.g. EEG electrode) on the protrusion.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an elastic, flexible, and/or articulated band between the frontpiece and a sidepiece (e.g. one of the two sidepieces), wherein an anterior end of the band is connected to the frontpiece, and wherein a posterior end of the band is slideably-connected to the sidepiece so that the posterior end can slide backward or forward along a portion of the length of the sidepiece; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an movable articulated arm, wherein a first end of the arm is attached to the front piece and a second end of the arm is attached to a sidepiece; and a brain activity sensor (e.g. EEG electrode) on the articulated arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a plurality of springs between the concavity and the sidepiece, wherein different springs extend out from the sidepiece at different angles; and a brain activity sensor (e.g. EEG electrode) which is attached to (the concavity of) the component.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible and/or bendable band between the frontpiece and a sidepiece, wherein a first end of the band is attached to the frontpiece and a second end of the band is attached to the sidepiece; a spring between the band and the sidepiece; and a brain activity sensor (e.g. EEG electrode) on the band. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; a spring between the movable arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; a spring between the movable arm and the sidepiece, wherein the central longitudinal axis of the spring is angled backward (at an acute angle) from the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm on a sidepiece, wherein the posterior end of the pivoting arm is movably connected to the sidepiece by a hinge or joint, and wherein the anterior end of the pivoting arm can extend out from the sidepiece; one or more springs between the pivoting arm and the sidepiece; and a brain activity sensor (e.g. EEG electrode) on the pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward and upward from the sidepiece; one or more springs between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; a spring between the middle of the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; a spring between the arm and the sidepiece, wherein the length and/or spring rate of the spring can be adjusted to change the distance by which the arm extends out from the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a spring between the movable arm and the sidepiece; and at least one brain activity sensor (e.g. electrode) on the movable arm. In an embodiment, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a first spring between the movable arm and the sidepiece; a second spring between the movable arm and the side piece, wherein the second spring has a higher spring rate than the first spring; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; an extension spring between the movable arm and the sidepiece; and at least one brain activity sensor (e.g. electrode) on the movable arm. In an embodiment, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a variable-pitch spring between the movable arm and the sidepiece; and at least one brain activity sensor (e.g. electrode) on the movable arm.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a compound movable arm on at least one of the sidepieces, wherein the compound movable arm further comprises a first section which is closer to the front of the eyewear and a second section which is farther from the front of the eyewear, and wherein the first and second sections are movably connected by a hinge or joint; at least one spring between the compound movable arm and the sidepiece; and at least one brain activity sensor (e.g. electrode) on the (first section of the) compound movable arm.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable arm on at least one of the sidepieces; a plurality of springs between the movable arm and the sidepiece; and at least one brain activity sensor (e.g. electrode) on the movable arm. In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; at least one spring between the pivoting arm and the sidepiece; and at least one brain activity sensor (e.g. electrode) on the (frontal half of the) pivoting arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a cylinder of compressible foam, wherein a first end of the cylinder is attached to a sidepiece (e.g. one of the two sidepieces) and a second end of the cylinder extends out toward a person's head from the sidepiece; and a brain activity sensor (e.g. EEG electrode) which is attached to (the second end of) the cylinder.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a frustum-shaped piece of compressible foam on (e.g. attached to or part of) the anterior third of a sidepiece; and a brain activity sensor (e.g. EEG electrode) on the piece of compressible foam. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a hemisphere of compressible foam, wherein a base of the hemisphere is attached to a sidepiece (e.g. one of the two sidepieces) and a dome of the hemisphere extends out toward a person's head from the sidepiece; and a brain activity sensor (e.g. EEG electrode) which is attached to (the dome of) the hemisphere.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; compressible foam between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward, upward, and inward (e.g. toward the person's head) from the sidepiece; compressible foam between the arm and the sidepiece; and a brain activity sensor (e.g. EEG electrode) on the arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; compressible foam between the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a triangular piece of compressible foam on (e.g. attached to or part of) the anterior half of a sidepiece; and a brain activity sensor (e.g. EEG electrode) on the piece of compressible foam. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate tensile protrusion which is part of, or attached to, a sidepiece (e.g. one of the two sidepieces); compressible foam between the protrusion and the sidepiece; and a brain activity sensor (e.g. EEG electrode) on the protrusion.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable foam pad on at least one of the sidepieces, wherein the foam pad has a first configuration between the sidepiece and the person's head, wherein the foam pad has a second configuration above the sidepiece, and wherein the foam pad is pivoted between the first configuration and the second configuration; and at least one brain activity sensor (e.g. electrode) on the foam pad.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting wedge-shaped component (e.g. foam pad) on at least one of the sidepieces, wherein the pivoting wedge-shaped component has a first configuration between the sidepiece and the person's head, wherein the pivoting wedge-shaped component has a second configuration above the sidepiece, and wherein the pivoting wedge-shaped component is pivoted between the first configuration and the second configuration; and at least one brain activity sensor (e.g. electrode) on the pivoting wedge-shaped component.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a rectangular foam pad on at least one of the sidepieces; and at least one brain activity sensor (e.g. electrode) on the rectangular foam pad. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an arcuate foam pad on (at least) one of the sidepieces, wherein a surface or perimeter of the foam pad which faces away from the side piece has a forward-skewed arcuate shape, wherein the shape comprises a curve with a Poisson distribution with a Lamba value between 5 and 10; and at least one brain activity electrode on the surface or perimeter of the foam pad which faces away from the side piece.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a multi-chamber expandable component on a sidepiece (e.g. one of the two sidepieces) which extends out toward a person's head from the sidepiece when expanded, wherein the multi-chamber expandable component is expanded by being filled with a flowable substance (e.g. a gas or fluid); and a brain activity sensor (e.g. EEG electrode) on the multi-chamber expandable chamber, wherein expansion of the multi-chamber expandable chamber presses the brain activity sensor onto the surface of the person's head.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward and upward from the sidepiece; an expandable (e.g. inflatable) chamber between the arm and the sidepiece; and a brain activity sensor (e.g. EEG electrode) on the arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; an expandable (e.g. inflatable) chamber between the middle of the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of expandable chambers on a sidepiece (e.g. one of the two sidepieces) which extend out toward a person's head from the sidepiece when expanded, wherein the chambers are expanded by being filled with a flowable substance (e.g. a gas or fluid); and a brain activity sensor (e.g. EEG electrode) on one or more of the chambers, wherein expansion of the one or more chambers presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate tensile protrusion which is part of, or attached to, a sidepiece (e.g. one of the two sidepieces); an expandable (e.g. inflatable) chamber between the protrusion and the sidepiece; and a brain activity sensor (e.g. EEG electrode) on the protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an hemispherical expandable chamber on a sidepiece (e.g. one of the two sidepieces) which extends out toward a person's head from the sidepiece when expanded, wherein the chamber is expanded by being filled with a flowable substance (e.g. a gas or fluid); and a brain activity sensor (e.g. EEG electrode) on the chamber, wherein expansion of the chamber presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of triangular inflatable chambers along a sidepiece; and one or more brain activity sensors (e.g. EEG electrodes) on one or more of the inflatable chambers.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a telescoping and pivoting arm between the concavity and the sidepiece; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pneumatic or hydraulic piston, wherein a first end of the piston is attached to a sidepiece (e.g. one of the two sidepieces) and a second end of the piston extends out toward a person's head from the sidepiece; and a brain activity sensor (e.g. EEG electrode) which is attached to (the second end of) the piston. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an outward-facing piston on a sidepiece; and a brain activity sensor (e.g. EEG electrode) on the piston.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch which is part of, or attached to, a sidepiece (e.g. one of the two sidepieces); a helically-threaded component between the flexible arch and the sidepiece, wherein rotation of the helically-threaded component changes the distance by which the flexible arch extends out from the sidepiece; and a brain activity sensor (e.g. EEG electrode) on the arch.

In an embodiment, a modular brain activity sensor component for eyewear (e.g. eyeglasses) can comprise: a clip-on component (e.g. pad) which is clipped, clamped, and/or hooked onto a sidepiece (e.g. temple) of eyewear (e.g. eyeglasses) by an anterior clip, clamp, and/or hook and a posterior clip, clamp, and/or hook; wherein there are one or more brain activity sensors (e.g. EEG electrodes) on the clip-on component. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a clip-on component (e.g. pad) which is clipped, clamped, and/or hooked onto a sidepiece (e.g. temple) in two places (e.g. by two clips, clamps, and/or hooks); and one or more brain activity sensors (e.g. EEG electrodes) on the clip-on component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a snap and/or other electrode receptor on the sidepiece; and a brain activity sensor (e.g. EEG electrode) which is connected to the snap and/or electrode receptor. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and a brain activity sensor (e.g. EEG electrode) which is attached to a sidepiece by anterior and posterior loops or bands.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an elastic band (e.g. band, strip, or strap) between the frontpiece and a sidepiece, wherein a first end of the band is attached to the frontpiece and a second end of the band is attached to the anterior third of the sidepiece; and a brain activity sensor (e.g. EEG electrode) on the band. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an elastic band (e.g. band, strip, or strap) between the frontpiece and a sidepiece, wherein a first end of the band is attached to the frontpiece and a second end of the band is attached to the sidepiece; and a brain activity sensor (e.g. EEG electrode) on the band.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece, a right sidepiece, and a left sidepiece; a flexible band (or strap), wherein a right end of the band is connected to the right sidepiece and a left end of the band is connected to the left sidepiece; and one or more brain activity sensors (e.g. EEG electrodes) on the flexible band. In an example, eyewear (e.g. eyeglasses) with brain activity sensors (e.g. EEG electrodes) can comprise: eyewear (e.g. eyeglasses) which is worn by a person, wherein the eyewear further comprises a frontpiece, a right sidepiece, and a left sidepiece; a transparent band (or strap), wherein a right end of the band is connected to the right sidepiece and a left end of the band is connected to the left sidepiece, and wherein the band spans the person's forehead; and one or more brain activity sensors (e.g. EEG electrodes) on the flexible band.

In another example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode further comprises a plurality of soft (e.g. Shore 00 value less than 70) electroconductive protrusions whose axes extend out from the sidepiece at different angles. In an embodiment, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode further comprises a plurality of soft (e.g. Shore 00 value less than 70) electroconductive protrusions with outwardly-concave longitudinal axes.

In another example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode has an obliquely-truncated pyramid shape, wherein a non-truncated base of the electrode faces toward the sidepiece, and wherein a truncated end of the electrode faces away from the sidepiece.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first average length, wherein a second subset of the plurality of electroconductive protrusions has a second average length, wherein the second average length is greater than the first average length, and wherein the second subset is closer to the rear of the person's head than the first subset.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first average length, wherein a second subset of the plurality of electroconductive protrusions has a second average length, wherein the second average length is greater than the first average length, and wherein the second subset is farther from the center of the electrode than the first subset.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from the eyewear toward the surface of the person's head. In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from the eyewear toward the surface of the person's head, wherein longitudinal axes of the protrusions are parallel to each other.

In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode has a truncated cylinder shape. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of frustum-shaped pads along a sidepiece; and one or more brain activity sensors (e.g. EEG electrodes) on one or more of the pads.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of soft electroconductive protrusions (e.g. teeth) which extend out from a sidepiece (e.g. one of the two sidepieces) toward the person's head, wherein the electroconductive protrusions record electrical brain activity (e.g. serving as a EEG electrode), wherein the electroconductive protrusions have a first configuration in which they are recessed into (openings, channels, grooves, or holes in) the sidepiece, and wherein the electroconductive protrusions have a second configuration in which they extend out from the sidepiece.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of soft electroconductive protrusions (e.g. teeth) which extend out from a sidepiece (e.g. one of the two sidepieces) toward the person's head; wherein the electroconductive protrusions are substantially perpendicular to the sidepiece and/or parallel to each other; and wherein the electroconductive protrusions record electrical brain activity (e.g. serving as a EEG electrode).

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of elastomeric (e.g. compressible) electroconductive protrusions (e.g. teeth) which extend out from a sidepiece (e.g. one of the two sidepieces) toward the person's head, wherein the electroconductive protrusions contact the surface of the person's head and record electromagnetic (e.g. EEG) brain activity.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at a MCN electrode location selected from the group consisting of T8, T10, and FT10; and a third brain activity sensor (e.g. EEG electrode) on the right sidepiece which is held on the person's head at a MCN electrode location wherein the third brain activity in on a pivoting arm which enables the third brain activity sensor to be held on the person's head at any of the MCN electrode locations selected from the group consisting of F10, F8, and F6.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at a MCN electrode location selected from the group consisting of T8, T10, and FT10; a third brain activity sensor (e.g. EEG electrode) a pivoting arm on the right sidepiece, wherein pivoting the arm changes the location of the third brain activity sensors between locations F10, F8, and F6.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) a pivoting arm on the right sidepiece, wherein pivoting the arm changes the location of the second brain activity sensor between locations AF8 and F6.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at a MCN electrode location selected from the group consisting of T7, T9, and FT9; a third brain activity sensor (e.g. EEG electrode) a pivoting arm on the left sidepiece, wherein pivoting the arm changes the location of the third brain activity sensors between locations F9, F7, and F5.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) a pivoting arm on the left sidepiece, wherein pivoting the arm changes the location of the second brain activity sensor between locations F9 and F7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) a pivoting arm on the left sidepiece, wherein pivoting the arm changes the location of the second brain activity sensor between locations AF7 and F5.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location TP8; and a third brain activity sensor (e.g. EEG electrode) on the right sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location FT10; and a third brain activity sensor (e.g. EEG electrode) on the right sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location FT8; and a third brain activity sensor (e.g. EEG electrode) on the right sidepiece which is held on the person's head at MCN electrode location AF8.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location F10; and a third brain activity sensor (e.g. EEG electrode) on the right sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location FT9; and a third brain activity sensor (e.g. EEG electrode) on the left sidepiece which is held on the person's head at MCN electrode location F8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at a MCN electrode location selected from the group consisting of T7, T9, FT9, and F9; and a third brain activity sensor (e.g. EEG electrode) on the left sidepiece which is held on the person's head at a MCN electrode location selected from the group consisting of FT7, F7, F5, AF7, AF3, and FP1.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location F5; and a third brain activity sensor (e.g. EEG electrode) on the left sidepiece which is held on the person's head at MCN electrode location F7.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location TP7; and a third brain activity sensor (e.g. EEG electrode) on the left sidepiece which is held on the person's head at MCN electrode location AF7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location TP7; and a third brain activity sensor (e.g. EEG electrode) on the left sidepiece which is held on the person's head at MCN electrode location FT9.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location T8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location FT10.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location F10.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location FP2. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location F8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location FT7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location FT9.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location AF3.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and a concentric-rings array of electroconductive elastomeric protrusions which extend out from a sidepiece (e.g. one of the two sidepieces) toward the surface of a person's head, wherein the protrusions record data concerning electrical activity of the person's brain. In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from an anterior half of a sidepiece (e.g. one of the two sidepieces) toward the surface of a person's head, wherein the protrusions record data concerning electrical activity of the person's brain from one or more MCN locations selected from the group consisting of: T7, T8, FT7, FT8, F5, F7, F8, F9, and AF7.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from a sidepiece (e.g. one of the two sidepieces) toward the surface of a person's head, wherein the protrusions record data concerning electrical activity of the person's brain. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out at different angles from a sidepiece (e.g. one of the two sidepieces) toward the surface of a person's head, wherein the angles vary with distance from the frontpiece, and wherein the protrusions record data concerning electrical activity of the person's brain.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from a sidepiece (e.g. one of the two sidepieces) toward the surface of a person's head, wherein the protrusions record data concerning electrical activity of the person's brain, and wherein the protrusions are made from an elastomeric polymer which has been doped, impregnated, and/or coated with electroconductive material.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from a sidepiece (e.g. one of the two sidepieces) toward the surface of a person's head, wherein the protrusions record data concerning electrical activity of the person's brain, and wherein the protrusions are made from an elastomeric polymer (e.g. SEBS) which has been doped, impregnated, and/or coated with electroconductive material (e.g. carbon black and/or silver particles). In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a concentric array of soft (e.g. Shore 00 value less than 70) electroconductive protrusions which are configured to extend out from the eyewear toward the surface of the person's head.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first Shore 00 level, wherein a second subset of the plurality of electroconductive protrusions has a second Shore 00 level, wherein the second level is greater than the first level, and wherein the second subset is closer to the center of the electrode than the first subset.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode is made from a thermoplastic elastomer which has been doped, impregnated, and/or coated with electroconductive particles. In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode is made from styrene-ethylene-butylene-styrene (SEBS) which has been doped, impregnated, and/or coated with carbon black and/or silver.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), and wherein an inward-facing (e.g. facing toward the head) surface of a sidepiece has an inward-facing bulge; and a brain activity sensor (e.g. EEG electrode) on the inward-facing bulge. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein (the longitudinal axis of) a sidepiece (e.g. one of the two sidepieces) has a horizontally sinusoidal and/or undulating shape; and one or more brain activity sensors (e.g. EEG electrodes) on the sidepiece.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein (the longitudinal axis of) a sidepiece (e.g. one of the two sidepieces) has a vertically and horizontally concave shape; and one or more brain activity sensors (e.g. EEG electrodes) on the sidepiece. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein a middle section of a sidepiece (e.g. one of the two sidepieces) has an upward wave; and a brain activity sensor (e.g. EEG electrode) on the wave of the sidepiece.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein an anterior third of a sidepiece (e.g. one of the two sidepieces) has an upward wave; and a brain activity sensor (e.g. EEG electrode) on the wave of the sidepiece. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein the posterior third of the sidepiece has a first vertical width, wherein the anterior third of the sidepiece has a second vertical width, and wherein the second vertical width is greater than twice the first vertical width; and a brain activity sensor (e.g. EEG electrode) on the anterior third of the sidepiece.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a bifurcation of a sidepiece into an upper branch and a lower branch on the anterior third of the sidepiece, wherein the opening or gap between the upper branch and the lower branch has stylized eye (or football) shape; and a brain activity sensor (e.g. EEG electrode) on the upper branch.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); wherein a sidepiece (e.g. one of the two sidepieces) bifurcates into an lower branch and an upper branch, wherein the lower branch is straight and spans the entire length of the sidepiece from an ear to the frontpiece, and wherein the upper branch is arcuate and spans from a non-end (e.g. middle) location on the anterior third of the sidepiece to a non-end (e.g. middle) location on the posterior third of the sidepiece; and one or more brain activity sensors (e.g. EEG electrodes) on the upper branch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein there are a plurality of pivoting arms between the flexible concave component and the sidepiece; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein there is a pivoting arm between the flexible concave component and the sidepiece; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch (e.g. band or strap) which is attached to a sidepiece (e.g. one of the two sidepieces) and curves upward from the sidepiece; and a brain activity sensor (e.g. EEG electrode) on the arch. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch (e.g. band or strap) which is attached to a sidepiece (e.g. one of the two sidepieces), wherein the arch has a first configuration in which the arch retracts into a recess (e.g. opening) in the sidepiece, and wherein the arch has a second configuration in which the arch extends out and upwards from the sidepiece; and a brain activity sensor (e.g. EEG electrode) on the arch.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible protrusion whose ends are movably attached to a sidepiece, wherein movement of the ends of the flexible protrusion closer together causes the flexible protrusion to extend further out from the sidepiece and movement of the ends of the flexible protrusion farther apart causes the flexible protrusion to extend out less from the sidepiece; and a brain activity sensor (e.g. EEG electrode) on the flexible protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position FT7.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position F7.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position AF3.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an attachable flexible arch whose ends are attached (e.g. clipped, clasped, or snapped) onto a sidepiece, wherein attaching the ends of the flexible arch closer together causes the flexible arch to extend further out from the sidepiece and attaching the ends of the flexible arch farther apart causes the flexible arch to extend out less from the sidepiece; and a brain activity sensor (e.g. EEG electrode) on the flexible arch.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a flexible concave protrusion (e.g. a flexible concave strip) on a sidepiece, wherein ends of the concave protrusion are attached to the sidepiece, and wherein a middle portion the concave protrusion extends out between 3 mm and 30 mm from the surface of the sidepiece toward the surface of the person's head; and a brain activity sensor (e.g. EEG electrode) on the concave protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a parabolic-shaped movable arm on a sidepiece (e.g. one of the two sidepieces), wherein the arm extends out from the sidepiece toward the surface of the person's head; and a brain activity sensor (e.g. EEG electrode) on the arm. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an undulating (e.g. sinusoidal) movable arm on a sidepiece (e.g. one of the two sidepieces), wherein the arm extends out from the sidepiece toward the surface of the person's head; and a brain activity sensor (e.g. EEG electrode) on the arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein one end of the pivoting arm is movably-connected (e.g. by a hinge or joint) to a sidepiece (e.g. one of the two sidepieces) and the other end of the pivoting arm free to extend out from the sidepiece, wherein the angle between the pivoting arm and the sidepiece is adjustable, wherein the pivoting arm can be locked at a selected angle between the pivoting arm and the sidepiece; and a brain activity sensor (e.g. EEG electrode) on the pivoting arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein a posterior end of the pivoting arm is movably attached (e.g. by a hinge or joint) to a sidepiece, wherein an anterior end of the pivoting arm has a first configuration in which it is (parallel to and) along the sidepiece, wherein the anterior end of the pivoting arm has a second configuration in which it extends out (at an angle) from the sidepiece toward the person's head, and wherein the pivoting arm is pivoted between the first configuration and the second configuration; and a brain activity sensor (e.g. EEG electrode) on the pivoting arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location T8. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location FT7.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location F7.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to the anterior third of a sidepiece (e.g. one of the two sidepieces), and wherein an anterior end of the arm extends out from the sidepiece; and a brain activity sensor (e.g. EEG electrode) on the anterior end of the arm. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable pad on at least one of the sidepieces, wherein the movable pad has a first configuration between the sidepiece and the person's head, wherein the movable pad has a second configuration above the sidepiece, and wherein the movable pad is moved (e.g. pivoted, rotated, and/or flipped up) between the first configuration and the second configuration; and at least one brain activity sensor (e.g. electrode) on the movable pad.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; a bellows-shaped chamber between the pivoting arm and the sidepiece; and at least one brain activity sensor (e.g. electrode) on the (frontal half of the) pivoting arm.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an arcuate arm on at least one of the sidepieces, wherein the arcuate arm has a first configuration between the sidepiece and the person's head, wherein the arcuate arm has a second configuration above the sidepiece, and wherein the arcuate arm is moved (e.g. pivoted, rotated, and/or flipped up) between the first configuration and the second configuration; and at least one brain activity sensor (e.g. electrode) on the arcuate arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a tension arm (e.g. prong) which extends out from the sidepiece toward the surface of the person's head, wherein a posterior end of the tension arm is attached to the sidepiece and an anterior end of the tension arm extends out from the sidepiece toward the surface of the person's head, wherein the length of the tension arm is between 10% and 30% of the length of the sidepiece; and a brain activity sensor (e.g. EEG electrode) on the tension arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate tensile protrusion which is part of, or attached to, a sidepiece (e.g. one of the two sidepieces), wherein an upper perimeter of the protrusion is not more than ¼ higher than an upper perimeter of the sidepiece, and wherein a lower perimeter of the protrusion is not more than ¼ lower than a lower perimeter of the sidepiece; and a brain activity sensor (e.g. EEG electrode) on the protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a concave flexible band which extends out from a sidepiece (e.g. one of the two sidepieces) toward the person's head, wherein ends of the band are slideably-connected to the sidepiece at different locations, wherein the band has a first configuration in which the ends are a first distance from each other and the band extends out from the sidepiece by a second distance, wherein the band has a second configuration in which the ends are a third distance from each other and the band extends out from the sidepiece by a fourth distance, wherein the third distance is less than the first distance, wherein the fourth distance is greater than the second distance, and wherein the band is changed from the first configuration to the second configuration by sliding one or both of the ends along the sidepiece; and one or more brain activity sensors (e.g. EEG electrodes) on the band.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a concave flexible band which extends out from a sidepiece (e.g. one of the two sidepieces) toward the person's head, wherein ends of the band are connected to the sidepiece at different locations, wherein one or both of the ends can slide along the sidepiece, and wherein sliding one or both of the ends along a track or channel on the sidepiece toward each other decreases the distance between the ends, which increases the distance by which the band extends out from the sidepiece toward the person's head; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch (e.g. band or strap) whose ends are slideably-attached to a sidepiece (e.g. one of the two sidepieces) and which curves upward from the sidepiece, wherein the arch has a first configuration which extends a first distance from the sidepiece and a second configuration which extends a second distance from the sidepiece, where the arch is changed from the first configuration to the second configuration by sliding one or both of the ends closer to each other; and a brain activity sensor (e.g. EEG electrode) on the arch.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) protrusion (e.g. arch) on a sidepiece (e.g. one of the two sidepieces), wherein an anterior portion (e.g. an anterior end) of the protrusion, a posterior portion (e.g. an posterior end) of the protrusion, or both are movably-attached (e.g. slideably-connected) to the sidepiece, wherein the protrusion has a first configuration in which there is a first distance between the anterior portion and the posterior portion and wherein the protrusion extends out a second distance from the sidepiece, wherein the protrusion has a second configuration in which there is a third distance between the anterior portion and the posterior portion and wherein the protrusion extends out a fourth distance from the sidepiece, wherein the third distance is less than the first distance, and wherein the fourth distance is greater than the second distance; an actuator, wherein the actuator moves the anterior portion and/or the posterior portion along a portion of the length of the sidepiece; and a brain activity sensor (e.g. EEG electrode) on the protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) protrusion (e.g. arch) on a sidepiece (e.g. one of the two sidepieces), wherein an anterior portion (e.g. an anterior end) of the protrusion, a posterior portion (e.g. an posterior end) of the protrusion, or both are movably-attached (e.g. slideably-connected) to the sidepiece, wherein the protrusion has a first configuration in which there is a first distance between the anterior portion and the posterior portion and wherein the protrusion extends out a second distance from the sidepiece, wherein the protrusion has a second configuration in which there is a third distance between the anterior portion and the posterior portion and wherein the protrusion extends out a fourth distance from the sidepiece, wherein the third distance is less than the first distance, wherein the fourth distance is greater than the second distance, and wherein the anterior portion and/or the posterior portion slide along a track (or channel) on the sidepiece; and a brain activity sensor (e.g. EEG electrode) on the protrusion.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a first component (e.g. first pad) which slides longitudinally along a sidepiece; a second component (e.g. second pad) which slides longitudinally along the sidepiece; wherein the first component and the second component have a first configuration in which they overlap by a first amount, wherein the first component and the second component have a second configuration in which they overlap by a second amount, wherein the second amount is greater than the first amount, and wherein the first component and the second component are changed from the first configuration to the second configuration by sliding the first component and/or the second component; and a brain activity sensor (e.g. EEG electrode) on the first component or the second component, wherein the brain activity sensor is a first distance from the sidepiece when the first and second components are in the first configuration, and wherein the brain activity sensor is a second distance from the sidepiece when the first and second components are in the second configuration.

In another example, eyewear (e.g. eyeglasses) with brain activity sensors (e.g. EEG electrodes) can comprise: eyewear (e.g. eyeglasses) which is worn by a person, wherein the eyewear further comprises two sidepieces (e.g. temples); an articulated arm, wherein a first end of the arm is attached to a first location (e.g. an anterior location) on a sidepiece and a second end of the arm is attached to second location (e.g. a posterior location) on the sidepiece; and a brain activity sensor (e.g. EEG electrode) on the articulated arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein there is a spring within the concavity; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein there is a spring between the flexible concave component and the sidepiece; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; a spring between the movable arm and the sidepiece, wherein the central longitudinal axis of the spring is perpendicular to the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; a plurality of springs with different sizes, lengths, and/or spring rates between the movable arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm on a sidepiece, wherein the posterior end of the pivoting arm is movably connected to the sidepiece by a hinge or joint, wherein the anterior end of the pivoting arm can extend out from the sidepiece; one or more springs between the pivoting arm and the sidepiece, wherein the tension, length, and/or spring rate of the one or more springs can be adjusted; and a brain activity sensor (e.g. EEG electrode) on the pivoting arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward and inward (e.g. toward the person's head) from the sidepiece; one or more springs between the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; a spring between the anterior end of the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate tensile protrusion which is part of, or attached to, a sidepiece (e.g. one of the two sidepieces); one or more springs between the protrusion and the sidepiece; and a brain activity sensor (e.g. EEG electrode) on the protrusion. In another example, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a first spring between the movable arm and the sidepiece; a second spring between the movable arm and the side piece; and at least one brain activity sensor (e.g. electrode) on the movable arm.

In an embodiment, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a first spring between the movable arm and the sidepiece whose longitudinal axis intersects the sidepiece at a first angle; a second spring between the movable arm and the side piece whose longitudinal axis intersects the sidepiece at a second angle, wherein the second spring is longer than the first spring; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a conical spring between the movable arm and the sidepiece; and at least one brain activity sensor (e.g. electrode) on the movable arm. In an example, eyewear (e.g. eyeglasses) with brain activity sensors (e.g. EEG electrodes) can comprise: eyewear (e.g. eyeglasses) which is worn by a person; a pivoting arm which is attached to a sidepiece (e.g. temple) of the eyewear; a spring between the pivoting arm and the sidepiece which pushes the pivoting arm away from the sidepiece; and at least one brain activity electrode on the pivoting arm, wherein the brain activity electrode further comprises a plurality of soft (e.g. Shore 00 value less than 70) electroconductive protrusions which are configured to extend out toward the surface of the person's head.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a flexible concave protrusion (e.g. a flexible concave strip) on a sidepiece, wherein ends of the concave protrusion are attached to the sidepiece and a middle portion the concave protrusion extends out between 3 mm and 30 mm from the sidepiece toward the surface of the person's head; a spring between the middle portion of the concave protrusion and the sidepiece; and a brain activity sensor (e.g. EEG electrode) on the concave protrusion.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable arm on at least one of the sidepieces, wherein one end of the movable arm is movably connected (e.g. by a hinge or joint) to the sidepiece; one or more compressive springs between the movable arm and the sidepiece; and at least one brain activity sensor (e.g. electrode) on the movable arm.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an articulated movable arm on at least one of the sidepieces, wherein the movable arm further comprises a first section which is closer to the front of the eyewear and a second section which is farther from the front of the eyewear, and wherein the first and second sections are movably connected by a hinge or joint; at least one spring between the movable arm and the sidepiece; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; compressible foam between the concavity and the sidepiece; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a frustum-shaped piece of compressible foam on (e.g. attached to or part of) the anterior half of a sidepiece; and a brain activity sensor (e.g. EEG electrode) on the piece of compressible foam. In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a oblate spheroid of compressible foam, wherein a first side of the oblate spheroid is attached to a sidepiece (e.g. one of the two sidepieces) and a second side of the oblate spheroid extends out toward a person's head from the sidepiece; and a brain activity sensor (e.g. EEG electrode) which is attached to (the second side of) the oblate spheroid.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward and upward from the sidepiece; compressible foam between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; compressible foam between the middle of the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a quadrilateral prism of compressible foam, wherein a first end of the quadrilateral prism is attached to a sidepiece (e.g. one of the two sidepieces) and a second end of the quadrilateral prism extends out toward a person's head from the sidepiece; and a brain activity sensor (e.g. EEG electrode) which is attached to (the second end of) the quadrilateral prism.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a wedge-shaped piece of compressible foam on (e.g. attached to or part of) the anterior third of a sidepiece; and a brain activity sensor (e.g. EEG electrode) on the piece of compressible foam. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a (coaxial) stack of disk-shaped pads on a sidepiece; and a brain activity sensor (e.g. EEG electrode) on the pads.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; compressible foam between the pivoting arm and the sidepiece; and at least one brain activity sensor (e.g. electrode) on the (frontal half of the) pivoting arm.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting wedge-shaped component (e.g. foam pad) on at least one of the sidepieces, wherein the pivoting wedge-shaped component has a first configuration between the sidepiece and the person's head, wherein the pivoting wedge-shaped component has a second configuration above the sidepiece, and wherein the pivoting wedge-shaped component is pivoted between the first configuration and the second configuration; and at least one brain activity sensor (e.g. electrode) on the pivoting wedge-shaped component, wherein the brain activity sensor is in contact with the person's head when the pivoting wedge-shaped component is in the first configuration.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an arcuate foam pad on (at least) one of the sidepieces, wherein a surface or perimeter of the foam pad which faces away from the side piece has a forward-skewed arcuate shape; and at least one brain activity electrode on the surface or perimeter of the foam pad which faces away from the side piece. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an oval-shaped foam pad on at least one of the sidepieces; and at least one brain activity sensor (e.g. electrode) on the oval-shaped foam pad.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm on a sidepiece, wherein the posterior end of the pivoting arm is movably connected to the sidepiece by a hinge or joint, and wherein the anterior end of the pivoting arm can extend out from the sidepiece; one or more inflatable chambers between the pivoting arm and the sidepiece; and a brain activity sensor (e.g. EEG electrode) on the pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward and inward (e.g. toward the person's head) from the sidepiece; an expandable (e.g. inflatable) chamber between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; an expandable (e.g. inflatable) chamber between the anterior end of the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a wedge-shaped expandable chamber on a sidepiece (e.g. one of the two sidepieces) which extends out toward a person's head from the sidepiece when expanded; and a brain activity sensor (e.g. EEG electrode) on the chamber, wherein expansion of the chamber presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an expandable chamber on a sidepiece (e.g. one of the two sidepieces) which extends out toward a person's head from the sidepiece when expanded; and a brain activity sensor (e.g. EEG electrode) on the expandable chamber, wherein expansion of the expandable chamber presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an inflatable chamber on a sidepiece (e.g. one of the two sidepieces) which extends out toward a person's head from the sidepiece when inflated; and a brain activity sensor (e.g. EEG electrode) on the inflatable chamber, wherein inflation of the inflatable chamber presses the brain activity sensor onto the surface of the person's head. In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of frustum-shaped inflatable chambers along a sidepiece; and one or more brain activity sensors (e.g. EEG electrodes) on one or more of the inflatable chambers.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a telescoping piston between the concavity and the sidepiece; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a telescoping arm, wherein a first end of the telescoping arm is attached to a sidepiece (e.g. one of the two sidepieces) and a second end of the telescoping arm extends out toward a person's head from the sidepiece; and a brain activity sensor (e.g. EEG electrode) which is attached to (the second end of) the telescoping arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a rotatable threaded member on (the anterior half of) a sidepiece, wherein the threaded member is inserted into (and/or through) a threaded opening in the sidepiece, and wherein rotation of the threaded member changes the extent to which the threaded member extends out from the sidepiece; and a brain activity sensor (e.g. EEG electrode) attached to (the end of) the threaded member.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch which is part of, or attached to, a sidepiece (e.g. one of the two sidepieces); a helically-threaded component between the flexible arch and the sidepiece, wherein the helically-threaded component fits into a helically-threaded opening in the sidepiece, and wherein rotation of the helically-threaded component changes the distance by which the flexible arch extends out from the sidepiece; and a brain activity sensor (e.g. EEG electrode) on the arch.

In an example, a modular brain activity sensor component for eyewear (e.g. eyeglasses) can comprise: a clip-on component (e.g. pad) which is clipped, clamped, and/or hooked onto a sidepiece (e.g. temple) of eyewear (e.g. eyeglasses); wherein there are one or more brain activity sensors (e.g. EEG electrodes) on the clip-on component. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a clip-on component (e.g. pad) which is clipped, clamped, and/or hooked onto a sidepiece (e.g. temple) by an anterior clip, clamp, and/or hook and a posterior clip, clamp, and/or hook; and one or more brain activity sensors (e.g. EEG electrodes) on the clip-on component. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and a brain activity sensor (e.g. EEG electrode) which is attached to a sidepiece.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible and/or bendable band between the frontpiece and a sidepiece, wherein a first end of the band is attached to the frontpiece and a second end of the band is attached to the sidepiece; and a brain activity sensor (e.g. EEG electrode) on the band.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an elastic band (e.g. band, strip, or strap) between the frontpiece and a sidepiece, wherein a first end of the band is attached to the frontpiece and a second end of the band is attached to the middle of the sidepiece; and a brain activity sensor (e.g. EEG electrode) on the band.

In an example, eyewear (e.g. eyeglasses) with brain activity sensors (e.g. EEG electrodes) can comprise: eyewear (e.g. eyeglasses) which is worn by a person; wherein the eyewear further comprises a frontpiece, a first sidepiece, and a second sidepiece; wherein the frontpiece further comprises a first rim around a first lens and a second rim around a second lens; a first arch (e.g. concave band) which is connected to the first rim, wherein the first arch is above the first rim and spans a first portion of the person's forehead; a first brain activity sensor (e.g. EEG electrode) on the first arch; a second arch (e.g. concave band) which is connected to the first rim, wherein the second arch is above the second rim and spans a second portion of the person's forehead; and a second brain activity sensor (e.g. EEG electrode) on the second arch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece, a right sidepiece, and a left sidepiece; a flexible band (or strap), wherein a right end of the band is connected to the right sidepiece and a left end of the band is connected to the left sidepiece, and wherein the band spans the person's forehead; and one or more brain activity sensors (e.g. EEG electrodes) on the flexible band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece, a right sidepiece, and a left sidepiece; an arcuate (e.g. undulating, wavy, and/or sinusoidal) band (or strap), wherein a right end of the band is connected to the right sidepiece and a left end of the band is connected to the left sidepiece; and one or more brain activity sensors (e.g. EEG electrodes) on the flexible band.

In another example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode further comprises a plurality of frustum-shaped soft (e.g. Shore 00 value less than 70) electroconductive protrusions. In an example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode further comprises a plurality of soft (e.g. Shore 00 value less than 70) electroconductive protrusions with concave longitudinal axes whose openings face away from a central axis of the electrode.

In an example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode has an obliquely-truncated rectangular prism shape, wherein a non-truncated base of the electrode faces toward the sidepiece, and wherein a truncated end of the electrode faces away from the sidepiece.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first average length, wherein a second subset of the plurality of electroconductive protrusions has a second average length, wherein the second average length is greater than the first average length, and wherein the second subset is closer to the front of the person's head than the first subset.

In an embodiment, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from the eyewear toward the surface of the person's head, and wherein protrusions which are closer to the front of the person's head are longer than protrusions which are farther from the person's head.

In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from a sidepiece (e.g. temple) of the eyewear toward the surface of the person's head, and wherein longitudinal axes of the protrusions intersect the sidepiece at acute posterior-facing angles.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from a sidepiece (e.g. temple) of the eyewear toward the surface of the person's head, wherein longitudinal axes of the protrusions are parallel to each other and non-orthogonal to the sidepiece.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of arcuate (e.g. semicircular) pads along a sidepiece; and one or more brain activity sensors (e.g. EEG electrodes) on one or more of the pads. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of cubic pads along a sidepiece; and one or more brain activity sensors (e.g. EEG electrodes) on one or more of the pads.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of soft electroconductive protrusions (e.g. teeth) which extend out from a sidepiece (e.g. one of the two sidepieces) toward the person's head, wherein the electroconductive protrusions record electrical brain activity (e.g. serving as a EEG electrode).

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of soft electroconductive protrusions (e.g. teeth) which extend out from a sidepiece (e.g. one of the two sidepieces) toward the person's head; wherein different electroconductive protrusions extend out from the sidepiece at different angles; and wherein the electroconductive protrusions record electrical brain activity (e.g. serving as a EEG electrode).

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of elastomeric (e.g. compressible) electroconductive protrusions (e.g. teeth) which extend out from a sidepiece (e.g. one of the two sidepieces) toward the person's head, wherein the electroconductive protrusions contact the surface of the person's head and serve as a brain activity sensor (e.g. EEG electrode).

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at a MCN electrode location selected from the group consisting of T7, T8, T9, and FT9; and a third brain activity sensor (e.g. EEG electrode) on the left sidepiece which is held on the person's head at a MCN electrode location wherein the third brain activity in on a pivoting arm which enables the third brain activity sensor to be held on the person's head at any of the MCN electrode locations selected from the group consisting of F9, F8, F7, and F5.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) a pivoting arm on the right sidepiece, wherein pivoting the arm changes the location of the second brain activity sensor between locations F10, F8, and F6.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) a pivoting arm on the right sidepiece, wherein pivoting the arm changes the location of the second brain activity sensor between locations FT10 and FT8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) a pivoting arm on the left sidepiece, wherein pivoting the arm changes the location of the second brain activity sensor between locations F9 and F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) a pivoting arm on the left sidepiece, wherein pivoting the arm changes the location of the second brain activity sensor between locations F9, F7, and F5.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location TP8; and a third brain activity sensor (e.g. EEG electrode) on the right sidepiece which is held on the person's head at MCN electrode location FT10.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location TP8; and a third brain activity sensor (e.g. EEG electrode) on the right sidepiece which is held on the person's head at MCN electrode location AF8.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location FT10; and a third brain activity sensor (e.g. EEG electrode) on the right sidepiece which is held on the person's head at MCN electrode location F6.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location F6; and a third brain activity sensor (e.g. EEG electrode) on the right sidepiece which is held on the person's head at MCN electrode location AF8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at a MCN electrode location selected from the group consisting of T7, T8, T9, FT9, and F9; and a third brain activity sensor (e.g. EEG electrode) on the left sidepiece which is held on the person's head at a MCN electrode location selected from the group consisting of FT7, F7, F8, F5, AF7, AF3, and FP1.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location F5; and a third brain activity sensor (e.g. EEG electrode) on the left sidepiece which is held on the person's head at MCN electrode location F8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location TP7; and a third brain activity sensor (e.g. EEG electrode) on the left sidepiece which is held on the person's head at MCN electrode location F7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location F9; and a third brain activity sensor (e.g. EEG electrode) on the left sidepiece which is held on the person's head at MCN electrode location F7.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location FT9; and a third brain activity sensor (e.g. EEG electrode) on the left sidepiece which is held on the person's head at MCN electrode location AF7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location TP7; and a third brain activity sensor (e.g. EEG electrode) on the left sidepiece which is held on the person's head at MCN electrode location F9.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location t10.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location F6.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location AF4.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location TP8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location T7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location AF7.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location F5.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location FP1.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and a linear array of electroconductive elastomeric protrusions which extend out from a sidepiece (e.g. one of the two sidepieces) toward the surface of a person's head, wherein the protrusions record data concerning electrical activity of the person's brain.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from a sidepiece (e.g. one of the two sidepieces) toward the surface of a person's head, wherein the protrusions record data concerning electrical activity of the person's brain from two or more MCN locations selected from the group consisting of: T7, T8, FT7, FT8, F5, F7, F8, F9, and AF7.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out orthogonally from a sidepiece (e.g. one of the two sidepieces) toward the surface of a person's head, wherein the protrusions record data concerning electrical activity of the person's brain. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric teeth which extend out from a sidepiece (e.g. one of the two sidepieces) toward the surface of a person's head, wherein the teeth record data concerning electrical activity of the person's brain.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from a sidepiece (e.g. one of the two sidepieces) toward the surface of a person's head, wherein the protrusions record data concerning electrical activity of the person's brain, and wherein the protrusions are made from an elastomeric polymer (e.g. a thermoplastic polymer) which has been doped, impregnated, and/or coated with electroconductive material (e.g. carbon and/or silver).

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an orthogonal (e.g. row and column) array of electroconductive elastomeric protrusions which extend out from a sidepiece (e.g. one of the two sidepieces) toward the surface of a person's head, wherein the protrusions record data concerning electrical activity of the person's brain.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first Shore 00 level, wherein a second subset of the plurality of electroconductive protrusions has a second Shore 00 level, and wherein the second level is greater than the first level.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first Shore 00 level, wherein a second subset of the plurality of electroconductive protrusions has a second Shore 00 level, wherein the second level is greater than the first level, and wherein the second subset is farther from the rear of the person's head than the first subset.

In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode is made from a thermoplastic elastomer which has been doped, impregnated, and/or coated with electroconductive particles (e.g. carbon black and/or silver particles). In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode is made from a thermoplastic elastomer (e.g. SEBS) which has been doped, impregnated, and/or coated with carbon black and/or silver.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein (the longitudinal axis of) a sidepiece (e.g. one of the two sidepieces) has a vertically sinusoidal and/or undulating shape; and one or more brain activity sensors (e.g. EEG electrodes) on the sidepiece. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein (the longitudinal axis of) a sidepiece (e.g. one of the two sidepieces) has a horizontally concave shape; and one or more brain activity sensors (e.g. EEG electrodes) on the sidepiece.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein a middle section a sidepiece (e.g. one of the two sidepieces) bifurcates into an upper branch and a lower branch; and a brain activity sensor (e.g. EEG electrode) on the upper branch. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein a middle section of a sidepiece (e.g. one of the two sidepieces) has an upward and inward wave; and a brain activity sensor (e.g. EEG electrode) on the wave of the sidepiece.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples), wherein an anterior third of a sidepiece (e.g. one of the two sidepieces) has an upward and inward wave; and a brain activity sensor (e.g. EEG electrode) on the wave of the sidepiece. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a bifurcation of a sidepiece into an upper branch and a lower branch on the anterior third of the sidepiece; and a brain activity sensor (e.g. EEG electrode) on the upper branch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an bifurcation on the anterior third of a sidepiece; and a brain activity sensor (e.g. EEG electrode) on the bifurcation. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); wherein a sidepiece (e.g. one of the two sidepieces) bifurcates into an lower branch and an upper branch, wherein the lower branch is straight and spans the entire length of the sidepiece from an ear to the frontpiece, and wherein the upper branch is arcuate and spans between 30% and 60% of the length of the sidepiece; and one or more brain activity sensors (e.g. EEG electrodes) on the upper branch.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein there are a plurality of pivoting arms between the flexible concave component and the sidepiece, wherein a first end of each pivoting arm is movably connected to the sidepiece (e.g. via a hinge or joint) and a second end of each pivoting arm is movably connected to the flexible concave component; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein there is a pivoting arm between the flexible concave component and the sidepiece, wherein a first end of the pivoting arm is movably connected to the sidepiece (e.g. via a hinge or joint) and a second end of the pivoting arm is movably connected to the flexible concave component; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible vertical arch (e.g. band or strap) which is attached to a sidepiece (e.g. one of the two sidepieces) and curves upward from the sidepiece; and a brain activity sensor (e.g. EEG electrode) on the arch. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch (e.g. band or strap) which is attached to a sidepiece (e.g. one of the two sidepieces), wherein the arch has a first configuration in which the arch retracts into a recess (e.g. opening) in the sidepiece, and wherein the arch has a second configuration in which the arch extends out and inward (e.g. toward the person's head) from the sidepiece; and a brain activity sensor (e.g. EEG electrode) on the arch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible band or strip, wherein a first end of the arm is attached to the front piece and a second end of the arm is attached to a sidepiece; and a brain activity sensor (e.g. EEG electrode) on the flexible band or strip. In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position F9.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) which is held by the arch at MCN electrode position F5.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch whose anterior and posterior ends are connected (e.g. attached) to a sidepiece (e.g. one of the two sidepieces), wherein the arch extends out from the sidepiece toward the surface of the person's head; and a brain activity sensor (e.g. EEG electrode) on the arch. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an upward arch (e.g. band or strap) which is attached to a sidepiece (e.g. one of the two sidepieces) and extends upward from the sidepiece; and a brain activity sensor (e.g. EEG electrode) on the arch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a linear (e.g. straight) movable arm on a sidepiece (e.g. one of the two sidepieces), wherein the arm extends out from the sidepiece toward the surface of the person's head; and a brain activity sensor (e.g. EEG electrode) on the arm. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate movable arm on a sidepiece (e.g. one of the two sidepieces), wherein the arm extends out from the sidepiece toward the surface of the person's head; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable arm on at least one of the sidepieces, wherein the movable arm has a first configuration it which it fits into a recess, channel, and/or opening in the sidepiece, wherein the movable arm has a second configuration in which it extends out from the sidepiece toward the person's head; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein one end of the pivoting arm is movably-connected (e.g. by a hinge or joint) to a sidepiece (e.g. one of the two sidepieces) and the other end of the pivoting arm free to extend out from the sidepiece, wherein the vertical angle between the pivoting arm and the sidepiece is adjustable, wherein the pivoting arm can be locked at a selected vertical angle between the pivoting arm and the sidepiece; and a brain activity sensor (e.g. EEG electrode) on the pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein a posterior end of the pivoting arm is movably attached (e.g. by a hinge or joint) to a sidepiece, wherein an anterior end of the pivoting arm has a first configuration in which it fits into a recess, opening, or channel in the sidepiece, wherein the anterior end of the pivoting arm has a second configuration in which it extends out from the recess, opening, or channel in the sidepiece toward the person's head, and wherein the pivoting arm is pivoted between the first configuration and the second configuration; and a brain activity sensor (e.g. EEG electrode) on the pivoting arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location T7.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location F9.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; and a brain activity sensor (e.g. EEG electrode) which is held by the arm at MCN electrode location F5.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to the anterior half of a sidepiece (e.g. one of the two sidepieces), and wherein an anterior end of the arm extends out from the sidepiece; and a brain activity sensor (e.g. EEG electrode) on the anterior end of the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; and a brain activity sensor (e.g. EEG electrode) on the arm. In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; and at least one brain activity sensor (e.g. electrode) on the (frontal half of the) pivoting arm.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; a pneumatic chamber between the pivoting arm and the sidepiece; and at least one brain activity sensor (e.g. electrode) on the (frontal half of the) pivoting arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a tension arm (e.g. prong) which extends out from the sidepiece toward the surface of the person's head, wherein a posterior end of the tension arm is attached to the sidepiece and an anterior end of the tension arm extends out from the sidepiece toward the surface of the person's head; and a brain activity sensor (e.g. EEG electrode) on the tension arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a tension arm (e.g. prong) which extends out from the sidepiece toward the surface of the person's head, wherein a posterior end of the tension arm is attached to the sidepiece and an anterior end of the tension arm extends out from the sidepiece toward the surface of the person's head, wherein the length of the tension arm is between 25% and 50% of the length of the sidepiece; and a brain activity sensor (e.g. EEG electrode) on the tension arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a band between the frontpiece and a sidepiece (e.g. one of the two sidepieces), wherein an anterior end of the band is connected to the frontpiece, and wherein a posterior end of the band is slideably-connected to the sidepiece so that the posterior end can slide backward or forward along a portion of the length of the sidepiece; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a concave flexible band which extends out from a sidepiece (e.g. one of the two sidepieces) toward the person's head, wherein ends of the band are connected to the sidepiece at locations which are between 1 and 3 apart from each other, wherein one or both of the ends can slide along the sidepiece, and wherein sliding one or both of the ends along a track or channel on the sidepiece toward each other decreases the distance between the ends, which increases the distance by which the band extends out from the sidepiece toward the person's head; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a concave flexible band which extends out from a sidepiece (e.g. one of the two sidepieces) toward the person's head, wherein ends of the band are connected to the sidepiece at different locations, wherein one or both of the ends can slide along the sidepiece, and wherein sliding one or both of the ends toward each other decreases the distance between the ends, which increases the distance by which the band extends out from the sidepiece toward the person's head; and one or more brain activity sensors (e.g. EEG electrodes) on the band.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible arch whose ends are slideably attached to a sidepiece, wherein sliding the ends of the flexible arch closer together causes the flexible arch to extend further out from the sidepiece and sliding the ends of the flexible arch farther apart causes the flexible arch to extend out less from the sidepiece; and a brain activity sensor (e.g. EEG electrode) on the flexible arch.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) protrusion (e.g. arch) on a sidepiece (e.g. one of the two sidepieces), wherein an anterior portion (e.g. an anterior end) of the protrusion, a posterior portion (e.g. an posterior end) of the protrusion, or both are movably-attached (e.g. slideably-connected) to the sidepiece, wherein the protrusion has a first configuration in which there is a first distance between the anterior portion and the posterior portion and wherein the protrusion extends out a second distance from the sidepiece, wherein the protrusion has a second configuration in which there is a third distance between the anterior portion and the posterior portion and wherein the protrusion extends out a fourth distance from the sidepiece, wherein the third distance is less than the first distance, and wherein the fourth distance is greater than the second distance; and a brain activity sensor (e.g. EEG electrode) on the protrusion.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an attachable flexible protrusion whose ends are attached (e.g. clipped, clasped, or snapped) onto a sidepiece, wherein attaching the ends of the flexible protrusion closer together causes the flexible protrusion to extend further out from the sidepiece and attaching the ends of the flexible protrusion farther apart causes the flexible protrusion to extend out less from the sidepiece; and a brain activity sensor (e.g. EEG electrode) on the flexible protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an articulated arm with a first segment and a second segment, wherein the first segment is attached to the front piece and the second segment is attached to a sidepiece, and wherein there is a hinge or joint between the first segment and the second segment; and a brain activity sensor (e.g. EEG electrode) on the first segment or the second segment.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a plurality of springs between the concavity and the sidepiece; and a brain activity sensor (e.g. EEG electrode) which is attached to (the concavity of) the component.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, wherein the flexible concave component has a first configuration in which the concavity extends a first distance out from the sidepiece, wherein the flexible concave component has a second configuration in which the concavity extends a second distance out from the sidepiece, and wherein the second distance is greater than the first distance; a spring between the concavity and the sidepiece; and a brain activity sensor (e.g. EEG electrode) which is attached to (the concavity of) the component.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein there are a plurality of springs between the flexible concave component and the sidepiece; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; a spring between the movable arm and the sidepiece, wherein the central longitudinal axis of the spring is angled forward (at an acute angle) from the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a movable arm on a sidepiece (e.g. one of the two sidepieces), wherein the arm extends out from the sidepiece toward the surface of the person's head; a plurality of springs between the movable arm and the sidepiece, wherein the spring intersect the sidepiece at different angles; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; one or more springs between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward, upward, and inward (e.g. toward the person's head) from the sidepiece; one or more springs between the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; a spring between the arm and the sidepiece; and a brain activity sensor (e.g. EEG electrode) on the arm. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an arcuate (flexible) arch which is part of, or attached to, a sidepiece (e.g. one of the two sidepieces); a plurality of springs between the flexible arch and the sidepiece; and a brain activity sensor (e.g. EEG electrode) on the arch.

In another example, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a first spring between the movable arm and the sidepiece; a second spring between the movable arm and the side piece, wherein the second spring is longer than the first spring; and at least one brain activity sensor (e.g. electrode) on the movable arm.

In an embodiment, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a compressive spring between the movable arm and the sidepiece; and at least one brain activity sensor (e.g. electrode) on the movable arm. In another example, eyewear (e.g. eyeglasses) with brain activity sensors (e.g. EEG electrodes) can comprise: eyewear (e.g. eyeglasses) which is worn by a person, wherein the eyewear further comprises right and left sidepieces (e.g. temples); a movable arm on at least one of the sidepieces; a torsion spring between the movable arm and the sidepiece; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person; a pivoting arm which is attached to a sidepiece (e.g. temple) of the eyewear; a spring between the pivoting arm and the sidepiece which pushes the pivoting arm away from the sidepiece; and at least one soft (e.g. Shore 00 value less than 70) brain activity electrode on the pivoting arm. In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable arm on at least one of the sidepieces, wherein one end of the movable arm is movably connected (e.g. by a hinge or joint) to the sidepiece; a plurality of springs with different spring rates between the movable arm and the sidepiece; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a movable arm on at least one of the sidepieces, wherein one end of the movable arm is movably connected (e.g. by a hinge or joint) to the sidepiece; a plurality of different length springs between the movable arm and the sidepiece; and at least one brain activity sensor (e.g. electrode) on the movable arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a cube of compressible foam, wherein a first side of the cube is attached to a sidepiece (e.g. one of the two sidepieces) and a second side of the cube extends out toward a person's head from the sidepiece; and a brain activity sensor (e.g. EEG electrode) which is attached to (the second side of) the cube.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a flexible concave component (e.g. arch, protrusion, band, strap, strip, or pad) which is attached to a sidepiece (e.g. one of the two sidepieces) and extends out toward a person's head from the sidepiece, wherein a concavity of the flexible concave component opens toward the sidepiece, and wherein there is compressible foam between the flexible concave component and the sidepiece; and a brain activity sensor (e.g. EEG electrode) which is attached to (the concavity of) the component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a frustum of compressible foam, wherein a base of the frustum is attached to a sidepiece (e.g. one of the two sidepieces) and an end of the frustum extends out toward a person's head from the sidepiece; and a brain activity sensor (e.g. EEG electrode) which is attached to (the end of) the frustum.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm on a sidepiece, wherein the posterior end of the pivoting arm is movably connected to the sidepiece by a hinge or joint, and wherein the anterior end of the pivoting arm can extend out from the sidepiece; one or more pieces of compressible foam between the pivoting arm and the sidepiece; and a brain activity sensor (e.g. EEG electrode) on the pivoting arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward and inward (e.g. toward the person's head) from the sidepiece; compressible foam between the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; compressible foam between the anterior end of the arm and the sidepiece; and a brain activity sensor (e.g. EEG electrode) on the arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a triangular piece of compressible foam on (e.g. attached to or part of) the anterior third of a sidepiece; and a brain activity sensor (e.g. EEG electrode) on the piece of compressible foam.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a wedge-shaped piece of compressible foam on (e.g. attached to or part of) the anterior half of a sidepiece; and a brain activity sensor (e.g. EEG electrode) on the piece of compressible foam. In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a (coaxial) stack of foam pads on a sidepiece; and a brain activity sensor (e.g. EEG electrode) on the pads.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting wedge-shaped component (e.g. foam pad) on at least one of the sidepieces, wherein the pivoting wedge-shaped component has a first configuration which is substantially in the same horizontal plane as the sidepiece (e.g. to the right or left of the sidepiece), wherein the pivoting wedge-shaped component has a second configuration which is substantially in the same vertical plane as the sidepiece (e.g. above the sidepiece), and wherein the pivoting wedge-shaped component is pivoted between the first configuration and the second configuration; and at least one brain activity sensor (e.g. electrode) on the pivoting wedge-shaped component.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting wedge-shaped component (e.g. foam pad) on at least one of the sidepieces, wherein the pivoting wedge-shaped component has a first configuration which is substantially in the same horizontal plane as the sidepiece (e.g. to the right or left of the sidepiece), wherein the pivoting wedge-shaped component has a second configuration which is substantially in the same vertical plane as the sidepiece (e.g. above the sidepiece), and wherein the pivoting wedge-shaped component is pivoted between the first configuration and the second configuration; and at least one brain activity sensor (e.g. electrode) on the pivoting wedge-shaped component, wherein the brain activity sensor is in contact with the person's head when the pivoting wedge-shaped component is in the first configuration.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); an arcuate foam pad on (at least) one of the sidepieces, wherein a surface or perimeter of the foam pad which faces away from the side piece has a forward-skewed arcuate shape, wherein the shape comprises a curve with a Poisson distribution with a Lamba value between 4 and 6; and at least one brain activity electrode on the surface or perimeter of the foam pad which faces away from the side piece.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a bellows-shaped and/or accordion-shaped expandable component on a sidepiece (e.g. one of the two sidepieces) which extends out toward a person's head from the sidepiece when expanded, wherein the component is expanded by being filled with a flowable substance (e.g. a gas or fluid); and a brain activity sensor (e.g. EEG electrode) on the chamber, wherein expansion of the chamber presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm can movably-extend out from the sidepiece; an expandable (e.g. inflatable) chamber between the arm and the sidepiece; and a brain activity sensor (e.g. EEG electrode) on the arm.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), wherein an anterior end of the arm extends outward, upward, and inward (e.g. toward the person's head) from the sidepiece; an expandable (e.g. inflatable) chamber between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm (or protrusion), wherein a posterior end of the arm is movably-attached (e.g. by a hinge or joint) to a sidepiece (e.g. one of the two sidepieces), and wherein an anterior end of the arm extends out from the sidepiece; an expandable (e.g. inflatable) chamber between the arm and the sidepiece; and a brain activity sensor (e.g. EEG electrode) on the arm.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a wedge-shaped expandable chamber on a sidepiece (e.g. one of the two sidepieces) which extends out toward a person's head from the sidepiece when expanded, wherein the chamber is expanded by being filled with a flowable substance (e.g. a gas or fluid); and a brain activity sensor (e.g. EEG electrode) on the chamber, wherein expansion of the chamber presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an expandable chamber on a sidepiece (e.g. one of the two sidepieces) which extends out toward a person's head from the sidepiece when expanded, wherein the expandable chamber is expanded by being filled with a flowable substance (e.g. a gas or fluid); and a brain activity sensor (e.g. EEG electrode) on the expandable chamber, wherein expansion of the expandable chamber presses the brain activity sensor onto the surface of the person's head.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of arcuate (e.g. semicircular) inflatable chambers along a sidepiece; and one or more brain activity sensors (e.g. EEG electrodes) on one or more of the inflatable chambers. In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a pivoting arm on at least one of the sidepieces, wherein the posterior end of the pivoting arm is connected to the sidepiece by a hinge or joint, wherein the frontal end of the pivoting arm is free to move relative to the sidepiece; an inflatable chamber between the pivoting arm and the sidepiece; and at least one brain activity sensor (e.g. electrode) on the (frontal half of the) pivoting arm.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of telescoping electroconductive protrusions (e.g. teeth) which extend out from a sidepiece (e.g. one of the two sidepieces) toward the person's head, wherein the electroconductive protrusions record electrical brain activity (e.g. serving as a EEG electrode), wherein the electroconductive protrusions have a first configuration in which they are recessed into (openings, channels, grooves, or holes in) the sidepiece, and wherein the electroconductive protrusions have a second configuration in which they extend out from the sidepiece. In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a telescoping protrusion on a sidepiece; and a brain activity sensor (e.g. EEG electrode) on the telescoping protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a rotatable threaded member on (the anterior half of) a sidepiece, wherein rotation of the threaded member changes the extent to which the threaded member extends out from the sidepiece; and a brain activity sensor (e.g. EEG electrode) attached to (the end of) the threaded member.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a flexible concave protrusion (e.g. flexible concave strip) on a sidepiece, wherein ends of the concave protrusion are attached to the sidepiece and a middle portion the concave protrusion extends out between 3 mm and 30 mm from the sidepiece toward the surface of the person's head; a rotatable threaded member between the middle portion of the concave protrusion and the sidepiece, wherein rotation of the threaded member changes the distance by which the concave protrusion extends out from the sidepiece; and a brain activity sensor (e.g. EEG electrode) on the concave protrusion.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a clip-on component (e.g. pad) which is clipped, clamped, and/or hooked onto a sidepiece (e.g. temple); and one or more brain activity sensors (e.g. EEG electrodes) on the clip-on component.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a pivoting arm, wherein one end of the pivoting arm is movably-connected (e.g. by a hinge or joint) to a sidepiece (e.g. one of the two sidepieces) and the other end of the pivoting arm free to extend out from the sidepiece; a snap and/or other electrode receptor on the pivoting arm; and a brain activity sensor (e.g. EEG electrode) which is connected to the snap and/or electrode receptor. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and a brain activity sensor (e.g. EEG electrode) which is attached to a sidepiece by anterior and posterior clips or clamps.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an articulated (e.g. jointed) band between the frontpiece and a sidepiece (e.g. one of the two sidepieces), wherein an anterior end of the band is connected to the frontpiece, and wherein a posterior end of the band is connected to the sidepiece; and one or more brain activity sensors (e.g. EEG electrodes) on the band. In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); an elastic band (e.g. band, strip, or strap) between the frontpiece and a sidepiece; and a brain activity sensor (e.g. EEG electrode) on the elastic band.

In an example, eyewear (e.g. eyeglasses) with brain activity sensors (e.g. EEG electrodes) can comprise: eyewear (e.g. eyeglasses) which is worn by a person; wherein the eyewear further comprises a frontpiece, a first sidepiece, and a second sidepiece; wherein the frontpiece further comprises a first rim around a first lens and a second rim around a second lens; a first arch (e.g. concave band) which is connected to the first rim, wherein the first arch is above a first eye and spans a first portion of the person's forehead; a first brain activity sensor (e.g. EEG electrode) on the first arch; a second arch (e.g. concave band) which is connected to the first rim, wherein the second arch is above a second eye and spans a second portion of the person's forehead; and a second brain activity sensor (e.g. EEG electrode) on the second arch.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece, a right sidepiece, and a left sidepiece; a flexible band (or strap), wherein a right end of the band is removably attached (e.g. by a clip or clasp) to the right sidepiece and a left end of the band is removably attached (e.g. by a clip or clasp) to the left sidepiece; and one or more brain activity sensors (e.g. EEG electrodes) on the flexible band.

In an embodiment, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode further comprises a plurality of soft (e.g. Shore 00 value less than 70) electroconductive protrusions which extend out from the sidepiece at different angles. In another example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode further comprises a plurality of soft (e.g. Shore 00 value less than 70) electroconductive protrusions with arcuate longitudinal axes.

In an example, eyewear (e.g. eyeglasses) with brain activity sensors (e.g. EEG electrodes) can comprise: eyewear (e.g. eyeglasses) which is worn by a person; and at least one brain activity electrode on a sidepiece (e.g. temple) of the eyewear, wherein the brain activity electrode has an obliquely-truncated cylinder shape, wherein a non-truncated base of the electrode faces toward the sidepiece, and wherein a truncated end of the electrode faces away from the sidepiece. In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first average length, wherein a second subset of the plurality of electroconductive protrusions has a second average length, and wherein the second average length is greater than the first average length.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first average length, wherein a second subset of the plurality of electroconductive protrusions has a second average length, wherein the second average length is greater than the first average length, and wherein the second subset is closer to the center of the electrode than the first subset.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from the eyewear toward the surface of the person's head, and wherein protrusions which are closer to the front of the person's head are less soft than protrusions which are farther from the person's head.

In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from a sidepiece (e.g. temple) of the eyewear toward the surface of the person's head, and wherein longitudinal axes of the protrusions are angled inward (toward the center of the person's head) and rearward (toward the rear of the person's head) as they extend out from the sidepiece.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of soft (e.g. Shore 00 value less than 70) electroconductive protrusions which are configured to extend out from a sidepiece (e.g. temple) of the eyewear toward the surface of the person's head, wherein longitudinal axes of the protrusions are parallel to each other and orthogonal to the sidepiece. In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples); a longitudinal (e.g. anterior to posterior) series of triangular pads along a sidepiece; and one or more brain activity sensors (e.g. EEG electrodes) on one or more of the pads.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of pivoting electroconductive protrusions (e.g. teeth) which extend out from a sidepiece (e.g. one of the two sidepieces) toward the person's head, wherein the electroconductive protrusions record electrical brain activity (e.g. serving as a EEG electrode), wherein the electroconductive protrusions have a first configuration in which they are recessed into (openings, channels, grooves, or holes in) the sidepiece, and wherein the electroconductive protrusions have a second configuration in which they extend out from the sidepiece.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of soft electroconductive protrusions (e.g. teeth) which extend out from a sidepiece (e.g. one of the two sidepieces) toward the person's head; wherein the electroconductive protrusions are made from a thermoplastic elastomer (e.g. TPU, SEBS, and/or PDMS) which has been doped, impregnated, and/or coated with electroconductive material (e.g. carbon and/or silver) and wherein the electroconductive protrusions record electrical brain activity (e.g. serving as a EEG electrode).

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); a plurality of elastomeric (e.g. compressible) protrusions (e.g. teeth) which extend out from a sidepiece (e.g. one of the two sidepieces) toward the person's head; and a brain activity sensor (e.g. EEG electrode) which is attached to the protrusions. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); five or more elastomeric (e.g. compressible) electroconductive protrusions (e.g. teeth) which extend out from a sidepiece (e.g. one of the two sidepieces) toward the person's head, wherein the electroconductive protrusions contact the surface of the person's head and serve as a brain activity sensor (e.g. EEG electrode).

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at a MCN electrode location selected from the group consisting of T7, T9, and FT9; and a third brain activity sensor (e.g. EEG electrode) on the left sidepiece which is held on the person's head at a MCN electrode location wherein the third brain activity in on a pivoting arm which enables the third brain activity sensor to be held on the person's head at any of the MCN electrode locations selected from the group consisting of F9, F7, and F5.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) a pivoting arm on the right sidepiece, wherein pivoting the arm changes the location of the second brain activity sensor between locations F10 and F8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at a MCN electrode location selected from the group consisting of T7, T8, T9, and FT9; a third brain activity sensor (e.g. EEG electrode) a pivoting arm on the left sidepiece, wherein pivoting the arm changes the location of the third brain activity sensors between locations F9, F8, F7, and F5.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) a pivoting arm on the left sidepiece, wherein pivoting the arm changes the location of the second brain activity sensor between locations F9, F8, F7, and F5.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) a pivoting arm on the left sidepiece, wherein pivoting the arm changes the location of the second brain activity sensor between locations FT9 and FT7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location TP8; and a third brain activity sensor (e.g. EEG electrode) on the right sidepiece which is held on the person's head at MCN electrode location F10.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location FT10; and a third brain activity sensor (e.g. EEG electrode) on the right sidepiece which is held on the person's head at MCN electrode location AF8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at a MCN electrode location selected from the group consisting of T8, T10, FT10, and F10; and a third brain activity sensor (e.g. EEG electrode) on the right sidepiece which is held on the person's head at a MCN electrode location selected from the group consisting of FT8, F8, F6, AF8, AF4, and FP2.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; a second brain activity sensor (e.g. EEG electrode) on the right sidepiece which is held on the person's head at MCN electrode location F6; and a third brain activity sensor (e.g. EEG electrode) on the right sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location TP7; and a third brain activity sensor (e.g. EEG electrode) on the left sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location F9; and a third brain activity sensor (e.g. EEG electrode) on the left sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location FT9; and a third brain activity sensor (e.g. EEG electrode) on the left sidepiece which is held on the person's head at MCN electrode location F7.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location FT7; and a third brain activity sensor (e.g. EEG electrode) on the left sidepiece which is held on the person's head at MCN electrode location AF7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location F5; and a third brain activity sensor (e.g. EEG electrode) on the left sidepiece which is held on the person's head at MCN electrode location AF7.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; a second brain activity sensor (e.g. EEG electrode) on the left sidepiece which is held on the person's head at MCN electrode location FT9; and a third brain activity sensor (e.g. EEG electrode) on the left sidepiece which is held on the person's head at MCN electrode location F5.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location FT8.

In an embodiment, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location F8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the right sidepiece which is held on the person's head (e.g. ear) at MCN electrode location A2; and a second brain activity sensor (e.g. EEG electrode) on the right sidepiece which is held on the person's head at MCN electrode location AF8.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location T8.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location F7.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location T9.

In another example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location F9.

In an example, eyewear (e.g. eyeglasses) with brain activity sensors (e.g. EEG electrodes) can comprise: eyewear (e.g. eyeglasses) which is worn by a person; wherein the eyewear further comprises right and left sidepieces (e.g. temples), wherein each of the right and left sidepieces spans from an ear to the front of the eyewear; a first brain activity sensor (e.g. EEG electrode) on the left sidepiece which is held on the person's head (e.g. ear) at MCN electrode location A1; and a second brain activity sensor (e.g. EEG electrode) on the left sidepiece which is held on the person's head at MCN electrode location TP7.

In another example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from an anterior half of a sidepiece (e.g. one of the two sidepieces) toward the surface of a person's head, wherein the protrusions extend out from the sidepiece at acute angles toward the rear of the person's head, and wherein the protrusions record data concerning electrical activity of the person's brain from one or more MCN locations selected from the group consisting of: T7, T8, FT7, FT8, F5, F7, F8, F9, and AF7.

In an embodiment, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from the anterior half of a sidepiece (e.g. one of the two sidepieces) toward the surface of a person's head, wherein the protrusions record data concerning electrical activity of the person's brain. In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out at different angles from a sidepiece (e.g. one of the two sidepieces) toward the surface of a person's head, wherein the protrusions record data concerning electrical activity of the person's brain.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of frustum-shaped electroconductive protrusions which extend out from a sidepiece (e.g. one of the two sidepieces) toward the surface of a person's head, wherein the protrusions record data concerning electrical activity of the person's brain.

In an example, eyewear (e.g. eyeglasses) with brain activity sensors (e.g. EEG electrodes) can comprise: eyewear (e.g. eyeglasses) which is worn by a person, wherein the eyewear further comprises a frontpiece and two sidepieces (e.g. temples); and an array of electroconductive elastomeric protrusions which extend out from a sidepiece (e.g. one of the two sidepieces) toward the surface of a person's head, wherein the protrusions record data concerning electrical activity of the person's brain, and wherein the protrusions are made from an elastomeric polymer (e.g. PDMS) which has been doped, impregnated, and/or coated with electroconductive material (e.g. carbon black and/or silver particles).

In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a circular array of soft (e.g. Shore 00 value less than 70) electroconductive protrusions which are configured to extend out from the eyewear toward the surface of the person's head.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first Shore 00 level, wherein a second subset of the plurality of electroconductive protrusions has a second Shore 00 level, wherein the second level is greater than the first level, and wherein the second subset is closer to the rear of the person's head than the first subset.

In an example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode further comprises a plurality of electroconductive protrusions which are configured to extend out from the eyewear toward the surface of the person's head, wherein a first subset of the plurality of electroconductive protrusions has a first Shore 00 level, wherein a second subset of the plurality of electroconductive protrusions has a second Shore 00 level, wherein the second level is greater than the first level, and wherein the second subset is farther from the center of the electrode than the first subset. In another example, eyewear (e.g. eyeglasses) with brain activity sensors (e.g. EEG electrodes) can comprise: eyewear (e.g. eyeglasses) which is worn by a person; and at least one brain activity electrode on the eyewear, wherein the brain activity electrode is made from a thermoplastic elastomer which has been doped, impregnated, and/or coated with carbon black and/or silver.

FIGS. 3 through 48 show different examples of “EEG glasses” (electroencephalographic eyewear). Relevant design and component variations discussed thus far in this disclosure or in other priority-linked disclosures can be applied to these, but are not repeated in the narratives accompanying each figure in order to reduce redundant content.

FIG. 3 shows a top-down view of an example of how this invention can be embodied in electroencephalographic eyewear comprising: (a) an eyewear frame which further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; wherein the flexible protrusion is contained within a three-dimensional space with an upper vertical boundary at the height of an Extended Upper Line, a lower vertical boundary at the height of an Extended Lower Line, an inner horizontal boundary at the horizontal location of an Extended Inside Line, and an outer horizontal boundary at the horizontal location of an Outside Perimeter Line; wherein an Upper Perimeter Line is a virtual straight line which most closely fits the upper perimeter of the anterior A″ of the selected side section; wherein the Extended Upper Line is a virtual straight line which is parallel to the Upper Perimeter Line and B″ above the Upper Perimeter Line; wherein a Lower Perimeter Line is the virtual straight line which is parallel to the Upper Perimeter Line and most closely fits the lower perimeter of the anterior A″ of the selected side section; wherein the Extended Lower Line is the virtual straight line which is parallel to the Upper Perimeter Line and C″ below the Lower Perimeter Line; wherein an Inside Perimeter Line is the virtual straight line which most closely fits the inside perimeter of the anterior A″ of the selected side section; wherein the Extended Inside Line is the virtual straight line which is parallel to the Inside Perimeter Line and D″ toward the person's head from the Inside Perimeter Line; wherein the Outside Perimeter Line is the virtual straight line which is parallel to the Upper Perimeter Line and most closely fits the outside perimeter of the anterior A″ of the selected side section; and wherein A″ is 6″ or less, B″ is ½″ or less, C″ is ½″ or less, and D″ is 2″ or less; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

FIG. 3 also shows a top-down view of electroencephalographic eyewear comprising: (a) an eyewear frame which further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; wherein the upper perimeter of the flexible protrusion is no more than ¼″ higher than the upper perimeter of the selected side section; and wherein the lower perimeter of the flexible protrusion is no more than ¼″ lower than the lower perimeter of the selected side section; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

FIG. 3 also shows a top-down view of electroencephalographic eyewear comprising: (a) an eyewear frame which further comprises a front section which is configured to span the front of a person's head, a first side section which is configured to span from a first ear to the front section, and a second side section which is configured to span from a second ear to the front section; (b) a flexible protrusion which is part of, or attached to, a selected side section; wherein the selected side section is selected from the group consisting of the first section and the second section; and wherein flexible protrusion is not visible from an outer side (lateral) view of the selected side section; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

With respect to specific components, FIG. 3 shows a top-down view of electroencephalographic eyewear comprising: (a) an eyewear frame which further comprises front section 3003 which is configured to span the front of a person's head, first side section 3001 which is configured to span from a first ear to front section 3003, and second side section 3002 which is configured to span from a second ear to front section 3003; (b) flexible protrusion 3005 which is part of, or attached to, selected side section 3001; (c) electromagnetic energy sensor 3004 which collects data concerning electromagnetic brain activity; wherein flexible protrusion 3005 is configured to hold electromagnetic energy sensor 3004 on the person's head; (d) energy source 3006; (e) data processor 3007; and (f) data transmitter and/or receiver 3008.

In this example, a flexible protrusion which extends inward from a side section of a frame to a person's head (holding the electromagnetic energy sensor on the person head) has a horizontal cross-section with a sinusoidal shape. In other examples, a protrusion which holds an electromagnetic energy sensor can have a shape selected from the group consisting of: bell curve, bellows, circular, conic, conic-section, cylindrical, egg-shaped, elliptical, frustal, half sinusoidal, half-bell curve, helical, kidney-bean, oval, parabolic, piston, pyramidic, quarter sinusoidal, rounded rectangular, rounded square, sinusoidal, spherical, S-shape, telescoping, triangular, and wedge shaped. In an example, a protrusion can be transparent or translucent so as to be less obvious.

In this example, a flexible protrusion is made from a compressible material such as compressible foam. In an example, a flexible protrusion can be made from a low durometer material. In an example, a flexible protrusion can be made from memory foam. In an example, a flexible member can be an inflatable member such as a balloon which is filled with a gas or liquid. In an example, the size and/or expansion of a flexible protrusion can be manually or automatically adjusted. In an example, the degree of pressure exerted by an electromagnetic energy sensor against a person's head can be manually or automatically adjusted by adjusting the size and/or expansion of a flexible protrusion which holds the sensor.

In an example, a flexible protrusion can have a first configuration in which the flexible protrusion extends a first distance from the inside perimeter of the side section of a frame for glasses or other eyewear, a second configuration in which the flexible protrusion extends a second distance from the inside perimeter of the side section of the frame, the second distance is greater than the first distance, and the protrusion can be reversibly moved from the first configuration to the second configuration. In an example, the flexible protrusion can be less visible in the first configuration and the electromagnetic energy sensor which it holds can be in closer electromagnetic communication with the person's body in the second configuration. In an example, a flexible protrusion can be reversibly changed from the first configuration to the second configuration by an action selected from the group consisting of: inflation; pneumatic pressure; magnetic attraction or repulsion; threaded rotation; spring motion; tensile movement; sliding one end of a flexible protrusion; movement of an elastic band; movement of telescoping member; and movement of a piston.

In an example, the location of a flexible protrusion on a side section of a frame can be adjusted forward and backward and/or up and down. In an example, the location of contact between an electromagnetic energy sensor and a person's head can be manually or automatically adjusted by adjusting the location of a flexible protrusion. In an example, a side section of a frame can have one or more tracks or channels along which a flexible protrusion can slide in order to adjust the location of the flexible protrusion. In an example, a side section of a frame can have one or more tracks or channels along which an end of a flexible protrusion can slide in order to adjust the location and/or extension of the flexible protrusion.

In this example, there is one flexible protrusion which holds one electromagnetic energy sensor. In another example, there can be multiple flexible protrusions, each of which holds one electromagnetic energy sensor. In another example, there can be one flexible protrusion which holds multiple electromagnetic energy sensors. In another example, there can be multiple flexible protrusions which hold multiple electromagnetic energy sensors.

In this example, the vertical boundaries of the perimeter of the flexible protrusion are entirely within the vertical boundaries of the perimeter of the side section. In another example, the vertical boundaries of the perimeter of the flexible protrusion can be slightly higher or lower than the vertical boundaries of the perimeter of the side section. In another example, the vertical boundaries of the perimeter of the flexible protrusion can be no more than ¼″ higher or lower than the vertical boundaries of the perimeter of the side section.

FIGS. 3 through 48 show various examples of how this invention can be embodied in EEG glasses or other electroencephalographic (EEG) eyewear. FIGS. 3 through 16 show examples of how this invention can be embodied in electroencephalographic eyewear comprising: (a) a frame for eyeglasses or other eyewear; wherein this frame further comprises a front section which is configured to span the front of a person's head, a first side section which is configured to span from a first ear to the front section, and a second side section which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, and then spans forward (3″-5″) along a relatively-straight longitudinal axis to connect to the front section; (b) a flexible protrusion which is part of, or attached to, a selected side section; (c) an electromagnetic energy sensor which collects data concerning electromagnetic brain activity; wherein the flexible protrusion is configured to hold the electromagnetic energy sensor on the person's head; (d) an energy source; (e) a data processor; and (f) a data transmitter and/or receiver.

FIG. 3 shows a top-down view of an example of how this invention can be embodied in EEG glasses or other electroencephalographic eyewear comprising: (a) an eyewear frame which further comprises front section 3003 which is configured to span the front of a person's head, first side section 3001 which is configured to span from a first ear to front section 3003, and second side section 3002 which is configured to span from a second ear to front section 3003; (b) sinusoidal or conic-section flexible protrusion 3005 which is part of, or attached to, selected side section 3001; (c) electromagnetic energy sensor 3004 which collects data concerning electromagnetic brain activity; wherein flexible protrusion 3005 is configured to hold electromagnetic energy sensor 3004 on the person's head; (d) energy source 3006; (e) data processor 3007; and (f) data transmitter and/or receiver 3008.

In an example, a protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In an example, flexible protrusion 3005 can be made from compressible foam. In an example, flexible protrusion 3005 can be filled with a gas or liquid. In an example, the extension of flexible protrusion 3005 can be adjusted by adjusting the amount and/or pressure of a gas or liquid inside it. Metric equivalents can also be used for inch measurements. Other relevant components and design variations discussed elsewhere in this disclosure or priority-linked disclosures can also be incorporated into this example.

FIG. 4 shows a top-down view of an example of how this invention can be embodied in EEG glasses or other electroencephalographic eyewear comprising: (a) an eyewear frame which further comprises front section 4003 which is configured to span the front of a person's head, first side section 4001 which is configured to span from a first ear to front section 4003, and second side section 4002 which is configured to span from a second ear to front section 4003; (b) elliptical or oval flexible protrusion 4005 which is part of, or attached to, selected side section 4001; (c) electromagnetic energy sensor 4004 which collects data concerning electromagnetic brain activity; wherein flexible protrusion 4005 is configured to hold electromagnetic energy sensor 4004 on the person's head; (d) energy source 4006; (e) data processor 4007; and (f) data transmitter and/or receiver 4008.

In an example, a protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In an example, flexible protrusion 4005 can be made from compressible foam. In an example, flexible protrusion 4005 can be filled with a gas or liquid. In an example, the extension of flexible protrusion 4005 can be adjusted by adjusting the amount and/or pressure of a gas or liquid inside it. Metric equivalents can also be used for inch measurements. Other relevant components and design variations discussed elsewhere in this disclosure or priority-linked disclosures can also be incorporated into this example.

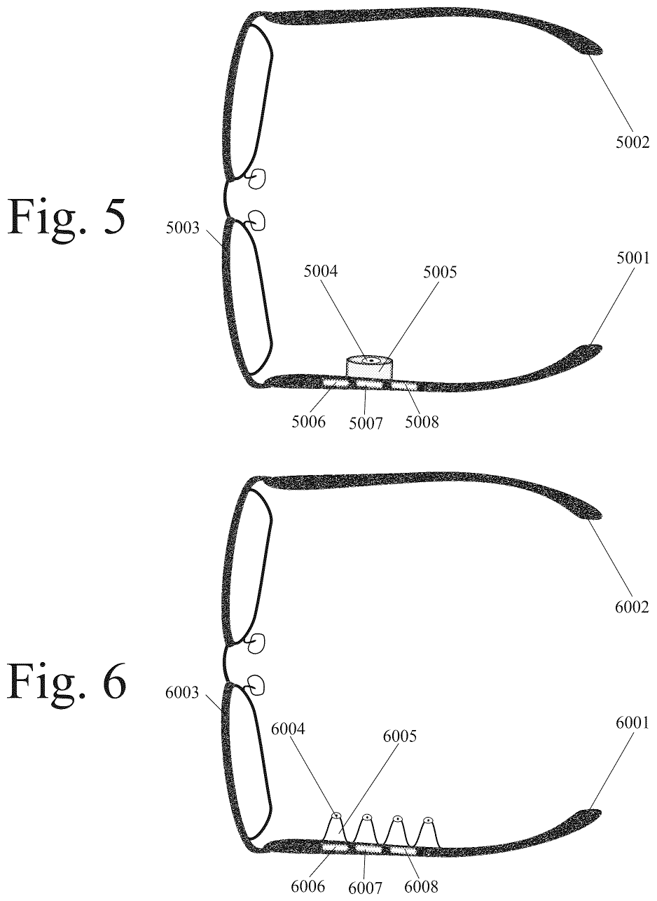

FIG. 5 shows a top-down view of an example of how this invention can be embodied in EEG glasses or other electroencephalographic eyewear comprising: (a) an eyewear frame which further comprises front section 5003 which is configured to span the front of a person's head, first side section 5001 which is configured to span from a first ear to front section 5003, and second side section 5002 which is configured to span from a second ear to front section 5003; (b) cylindrical or rectangular protrusion 5005 which is part of, or attached to, selected side section 5001; (c) electromagnetic energy sensor 5004 which collects data concerning electromagnetic brain activity; wherein cylindrical protrusion 5005 is configured to hold electromagnetic energy sensor 5004 on the person's head; (d) energy source 5006; (e) data processor 5007; and (f) data transmitter and/or receiver 5008.

In an example, a protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In an example, protrusion 5005 can be made from compressible foam. In an example, protrusion 5005 can be filled with a gas or liquid. In an example, the extension of protrusion 5005 can be adjusted by adjusting the amount and/or pressure of a gas or liquid inside it. Metric equivalents can also be used for inch measurements. Other relevant components and design variations discussed elsewhere in this disclosure or priority-linked disclosures can also be incorporated into this example.

FIG. 6 shows a top-down view of an example of how this invention can be embodied in EEG glasses or other electroencephalographic eyewear comprising: (a) an eyewear frame which further comprises front section 6003 which is configured to span the front of a person's head, first side section 6001 which is configured to span from a first ear to front section 6003, and second side section 6002 which is configured to span from a second ear to front section 6003; (b) a plurality of arcuate (sinusoidal) protrusions, including 6005, which are part of, or attached to, selected side section 6001; (c) a plurality of electromagnetic energy sensors, including 6004, which collect data concerning electromagnetic brain activity; wherein the plurality of protrusions are configured to hold the plurality of electromagnetic energy sensors on the person's head; (d) energy source 6006; (e) data processor 6007; and (f) data transmitter and/or receiver 6008.

In an example, protrusions can be part of, or attached to, a selected side section, wherein the upper perimeters of the protrusions are not more than ¼″ higher than the upper perimeter of the selected side section, and wherein the lower perimeter of the protrusion is not more than ¼″ lower than the lower perimeter of the selected side section. In an example, protrusions can be made from compressible foam. In an example, protrusions can be filled with a gas or liquid. In an example, extension of the protrusions can be adjusted by adjusting the amounts and/or pressures of gases or liquids inside them. Metric equivalents can also be used for inch measurements. Other relevant components and design variations discussed elsewhere in this disclosure or priority-linked disclosures can also be incorporated into this example.

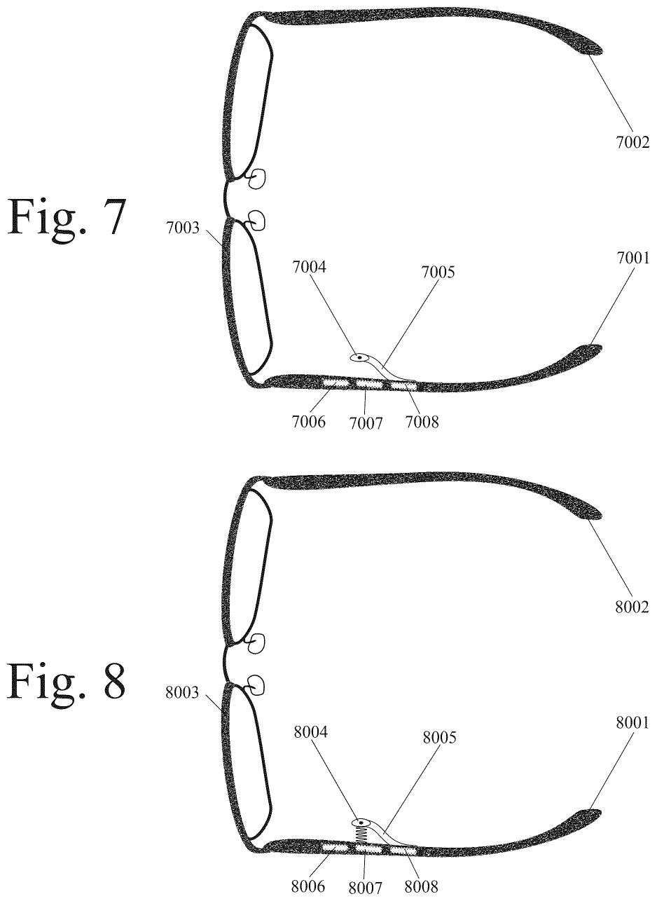

FIG. 7 shows a top-down view of an example of how this invention can be embodied in EEG glasses or other electroencephalographic eyewear comprising: (a) an eyewear frame which further comprises front section 7003 which is configured to span the front of a person's head, first side section 7001 which is configured to span from a first ear to front section 7003, and second side section 7002 which is configured to span from a second ear to front section 7003; (b) an arcuate tensile protrusion 7005 which is part of, or attached to, selected side section 7001; (c) an electromagnetic energy sensor 7004 which collects data concerning electromagnetic brain activity; wherein the protrusion is configured to hold the electromagnetic energy sensor on the person's head; (d) energy source 7006; (e) data processor 7007; and (f) data transmitter and/or receiver 7008.

In an example, the protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example, the posterior (rear) end of the protrusion is directly attached to the selected side section and the anterior (front) end of the protrusion is not directly attached to the selected side section. In an example, the protrusion can have a half-sinusoidal shape. Metric equivalents can also be used for inch measurements. Other relevant components and design variations discussed elsewhere in this disclosure or priority-linked disclosures can also be incorporated into this example.

FIG. 8 shows a top-down view of an example of how this invention can be embodied in EEG glasses or other electroencephalographic eyewear comprising: (a) an eyewear frame which further comprises front section 8003 which is configured to span the front of a person's head, first side section 8001 which is configured to span from a first ear to front section 8003, and second side section 8002 which is configured to span from a second ear to front section 8003; (b) an arcuate tensile protrusion 8005 which is part of, or attached to, selected side section 8001; (c) an electromagnetic energy sensor 8004 which collects data concerning electromagnetic brain activity; wherein the protrusion is configured to hold the electromagnetic energy sensor on the person's head; (d) energy source 8006; (e) data processor 8007; and (f) data transmitter and/or receiver 8008.

FIG. 8 can also be described as showing EEG glasses or other electroencephalographic eyewear which comprise: an eyewear frame, wherein the eyewear frame further comprises a frontpiece which is configured to span the front of a person's head, a first sidepiece which is configured to span from a first ear to the frontpiece, and a second sidepiece which is configured to span from a second ear to the frontpiece; an arcuate tensile protrusion which is part of, or attached to, a selected sidepiece; an electrode or other electromagnetic energy sensor on the protrusion which collects data concerning brain activity; an energy source; a data processor; and a data transmitter and/or receiver. In this example, there is a spring between the protrusion and the selected sidepiece. In this example, one end of the protrusion is attached to the sidepiece and the other end of the protrusion is free to extend out from the sidepiece. In this example, a posterior end of the protrusion is attached to the sidepiece and an anterior end of the protrusion is free to extend out from the sidepiece.

In an example, a protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example, there is a spring between the anterior (front) end of the arcuate protrusion and the selected side section. Metric equivalents can also be used for inch measurements. Other relevant components and design variations discussed elsewhere in this disclosure or priority-linked disclosures can also be incorporated into this example.

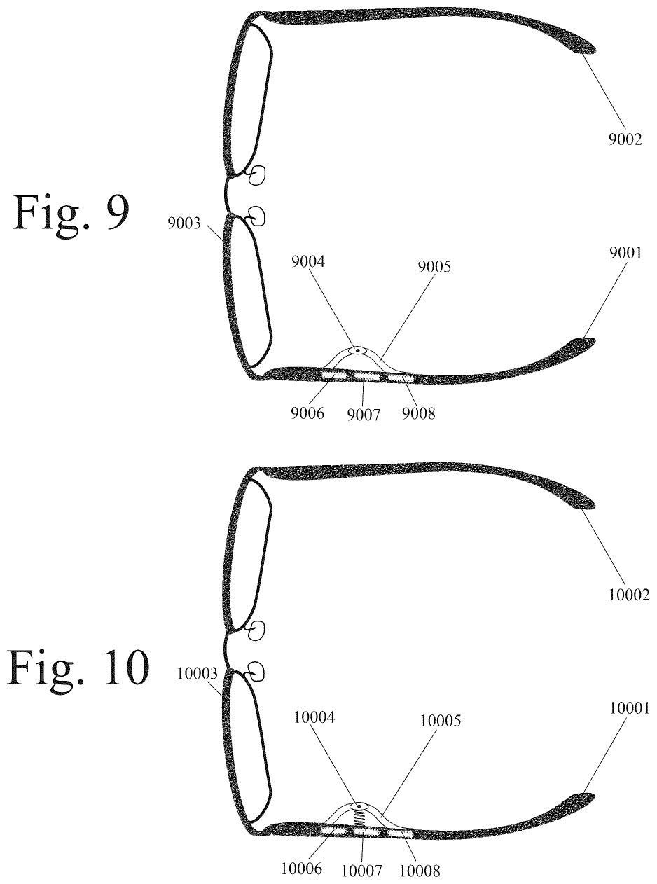

FIG. 9 shows a top-down view of an example of how this invention can be embodied in EEG glasses or other electroencephalographic eyewear comprising: (a) an eyewear frame which further comprises front section 9003 which is configured to span the front of a person's head, first side section 9001 which is configured to span from a first ear to front section 9003, and second side section 9002 which is configured to span from a second ear to front section 9003; (b) an arcuate tensile protrusion 9005 which is part of, or attached to, selected side section 9001; (c) an electromagnetic energy sensor 9004 which collects data concerning electromagnetic brain activity; wherein the protrusion is configured to hold the electromagnetic energy sensor on the person's head; (d) energy source 9006; (e) data processor 9007; and (f) data transmitter and/or receiver 9008.

In an example, a protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example, both the posterior (rear) end and the anterior (front) end of the arcuate tensile protrusion are directly attached to the selected side section, but the middle of the arcuate tensile protrusion between these ends is not directly attached to the selected side section. Metric equivalents can also be used for inch measurements. Other relevant components and design variations discussed elsewhere in this disclosure or priority-linked disclosures can also be incorporated into this example.

FIG. 10 shows a top-down view of an example of how this invention can be embodied in EEG glasses or other electroencephalographic eyewear comprising: (a) an eyewear frame which further comprises front section 10003 which is configured to span the front of a person's head, first side section 10001 which is configured to span from a first ear to front section 10003, and second side section 10002 which is configured to span from a second ear to front section 10003; (b) arcuate tensile protrusion 10005 which is part of, or attached to, selected side section 10001; (c) electromagnetic energy sensor 10004 which collects data concerning electromagnetic brain activity; wherein the protrusion is configured to hold the electromagnetic energy sensor on the person's head; (d) energy source 10006; (e) data processor 10007; and (f) data transmitter and/or receiver 10008.

FIG. 10 can also be described as showing EEG glasses or other electroencephalographic eyewear which comprise: an eyewear frame which further comprises a front section which is configured to span the front of a person's head, a first side section which is configured to span from a first ear to the front section, and a second side section which is configured to span from a second ear to the front section; an arcuate protrusion which is part of, or attached to, a selected side section; an electromagnetic energy sensor which collects data concerning electromagnetic brain activity, wherein the protrusion is configured to hold the electromagnetic energy sensor on the person's head; an energy source; a data processor; and a data transmitter and/or receiver. In this example, both ends of the protrusion are attached to the sidepiece.

In an example, a protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example: both the posterior (rear) end and the anterior (front) end of the arcuate tensile protrusion are directly attached to the selected side section; and there is a spring between the middle portion of the arcuate tensile protrusion and the selected side section. Metric equivalents can also be used for inch measurements. Other relevant components and design variations discussed elsewhere in this disclosure or priority-linked disclosures can also be incorporated into this example.

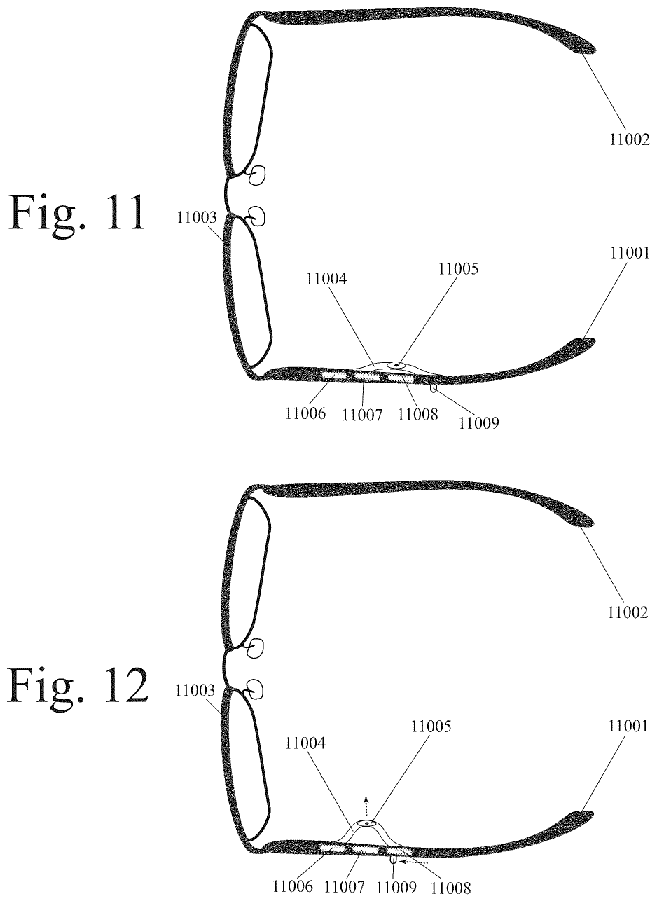

FIGS. 11 and 12 show two sequential top-down views of an example showing how a flexible protrusion which holds an electromagnetic energy sensor can be adjusted. This example comprises: (a) an eyewear frame which further comprises front section 11003 which is configured to span the front of a person's head, first side section 11001 which is configured to span from a first ear to front section 11003, and second side section 11002 which is configured to span from a second ear to front section 11003; (b) flexible protrusion 11004 which is part of, or attached to, selected side section 11001; (c) electromagnetic energy sensor 11005 which collects data concerning electromagnetic brain activity; wherein the protrusion is configured to hold the electromagnetic energy sensor on the person's head; (d) energy source 11006; (e) data processor 11007; (f) data transmitter and/or receiver 11008; and (g) sliding knob 11009.

In this example, flexible protrusion 11004 has a first configuration in which it extends a first distance from the selected side section toward a person's head, has a second configuration in which it extends a second distance from the selected side section toward the person's head, the second distance is greater than the first distance, and the flexible protrusion can be reversibly adjusted (moved or changed) from the first configuration to the second configuration. FIG. 11 shows the flexible protrusion in its first configuration and FIG. 12 shows the flexible protrusion in its second configuration.

FIGS. 11 and 12 can also be described as showing EEG glasses or other electroencephalographic eyewear which comprise: an eyewear frame which further comprises a front section which is configured to span the front of a person's head, a first side section which is configured to span from a first ear to the front section, and a second side section which is configured to span from a second ear to the front section; an arcuate protrusion which is part of, or attached to, a selected side section; an electromagnetic energy sensor which collects data concerning electromagnetic brain activity, wherein the protrusion is configured to hold the electromagnetic energy sensor on the person's head; an energy source; a data processor; and a data transmitter and/or receiver. In this example, both ends of the protrusion are attached to the sidepiece.

In this example, the protrusion has a first configuration which extends a first distance out from the selected sidepiece toward a person's head, the protrusion has a second configuration which extends a second distance out from the selected sidepiece toward the person's head, and the second distance is greater than the first distance. In this example, the protrusion can be changed from the first configuration to the second configuration by moving one or both of the ends of the protrusion closer to each other, causing the protrusion to bulge out towards toward the person's head. In an example, one or both of the ends of the protrusion can slide along the sidepiece. In an example, one or both of the ends of the protrusion can slide along a track or channel on the sidepiece.

In this example, the flexible protrusion is adjusted (moved or changed) from its first configuration to its second configuration by manually moving sliding knob 11009, which moves one end of the flexible protrusion closer to the other end of the flexible protrusion and causes the middle of the flexible protrusion to bulge (further) towards toward the person's head. In this example, one end of the flexible protrusion is slid closer to the other end along a track or channel, causing the middle of the flexible protrusion to bulge (further) outwards toward the person's head. In another example, a flexible protrusion can be automatically moved from its first configuration to its second configuration by an actuator. Metric equivalents can also be used for inch measurements. Other relevant components and design variations discussed elsewhere in this disclosure or priority-linked disclosures can also be incorporated into this example.

FIG. 13 shows a top-down view of an example of how this invention can be embodied in EEG glasses or other electroencephalographic eyewear comprising: (a) an eyewear frame which further comprises front section 13003 which is configured to span the front of a person's head, first side section 13001 which is configured to span from a first ear to front section 13003, and second side section 13002 which is configured to span from a second ear to front section 13003; (b) a folded and/or pleated protrusion 13004 which is part of, or attached to, selected side section 13001; (c) an electromagnetic energy sensor 13005 which collects data concerning electromagnetic brain activity; wherein the protrusion is configured to hold the electromagnetic energy sensor on the person's head; (d) energy source 13006; (e) data processor 13007; and (f) data transmitter and/or receiver 13008.

In an example, the protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In an example, a folded and/or pleated protrusion can be extended (further) from the side section toward the person's head by filling the protrusion with a gas or liquid. In this example, the folded and/or pleated protrusion is shaped like a bellows or accordion. Metric equivalents can also be used for inch measurements. Other relevant components and design variations discussed elsewhere in this disclosure or priority-linked disclosures can also be incorporated into this example.

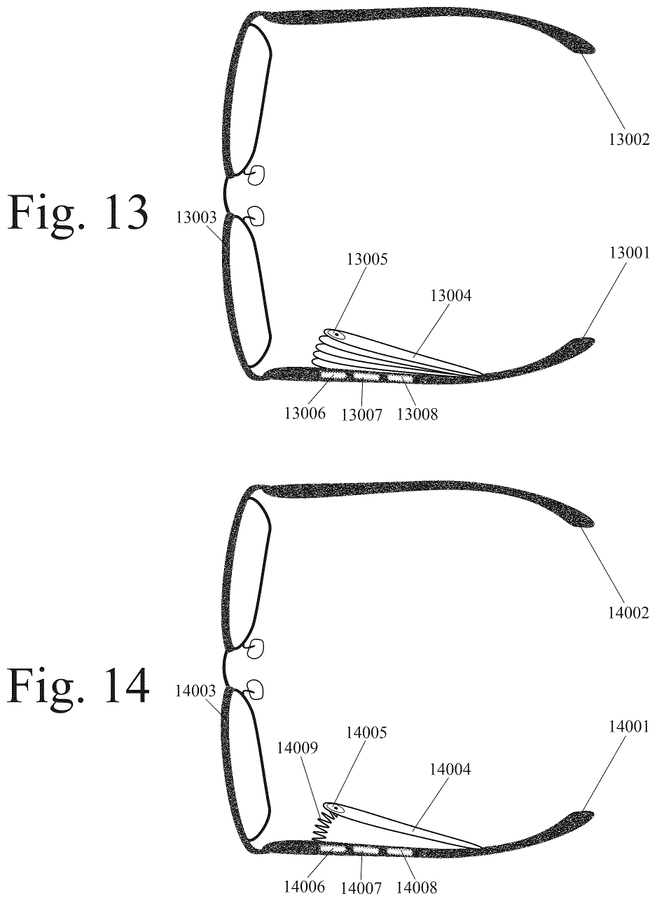

FIG. 14 shows a top-down view of an example of how this invention can be embodied in EEG glasses or other electroencephalographic eyewear comprising: (a) an eyewear frame which further comprises front section 14003 which is configured to span the front of a person's head, first side section 14001 which is configured to span from a first ear to front section 14003, and second side section 14002 which is configured to span from a second ear to front section 14003; (b) a pivoting protrusion 14004 which is part of, or attached to, selected side section 14001; (c) an electromagnetic energy sensor 14005 which collects data concerning electromagnetic brain activity; wherein the protrusion is configured to hold the electromagnetic energy sensor on the person's head; (d) energy source 14006; (e) data processor 14007; and (f) data transmitter and/or receiver 14008.

In an example, the protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example, there is a spring 14009 between the pivoting protrusion and the side section. Metric equivalents can also be used for inch measurements. Other relevant components and design variations discussed elsewhere in this disclosure or priority-linked disclosures can also be incorporated into this example.

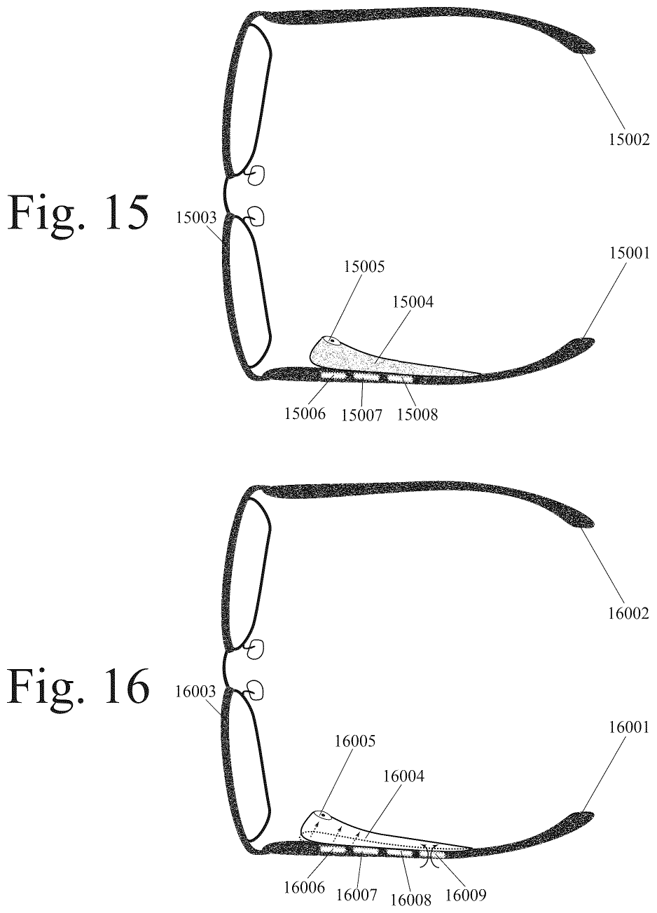

FIG. 15 shows a top-down view of an example of how this invention can be embodied in EEG glasses or other electroencephalographic eyewear comprising: (a) an eyewear frame which further comprises front section 15003 which is configured to span the front of a person's head, first side section 15001 which is configured to span from a first ear to front section 15003, and second side section 15002 which is configured to span from a second ear to front section 15003; (b) a wedge-shaped flexible protrusion 15004 which is part of, or attached to, selected side section 15001; (c) an electromagnetic energy sensor 15005 which collects data concerning electromagnetic brain activity; wherein the protrusion is configured to hold the electromagnetic energy sensor on the person's head; (d) energy source 15006; (e) data processor 15007; and (f) data transmitter and/or receiver 15008.

In an example, the protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example, the wedge-shaped protrusion is made from compressible foam. Metric equivalents can also be used for inch measurements. Other relevant components and design variations discussed elsewhere in this disclosure or priority-linked disclosures can also be incorporated into this example.

FIG. 16 shows a top-down view of an example of how this invention can be embodied in EEG glasses or other electroencephalographic eyewear comprising: (a) an eyewear frame which further comprises front section 16003 which is configured to span the front of a person's head, first side section 16001 which is configured to span from a first ear to front section 16003, and second side section 16002 which is configured to span from a second ear to front section 16003; (b) a wedge-shaped flexible protrusion 16004 which is part of, or attached to, selected side section 16001; (c) an electromagnetic energy sensor 16005 which collects data concerning electromagnetic brain activity; wherein the protrusion is configured to hold the electromagnetic energy sensor on the person's head; (d) energy source 16006; (e) data processor 16007; (f) data transmitter and/or receiver 16008; and pump 16009.

In an example, the protrusion can be part of, or attached to, a selected side section, wherein the upper perimeter of the protrusion is not more than ¼″ higher than the upper perimeter of the selected side section, and wherein the lower perimeter of the protrusion is not more than ¼″ lower than the lower perimeter of the selected side section. In this example, the wedge-shaped protrusion can be inflated or deflated by activation of pump 16009. Metric equivalents can also be used for inch measurements. Other relevant components and design variations discussed elsewhere in this disclosure or priority-linked disclosures can also be incorporated into this example.

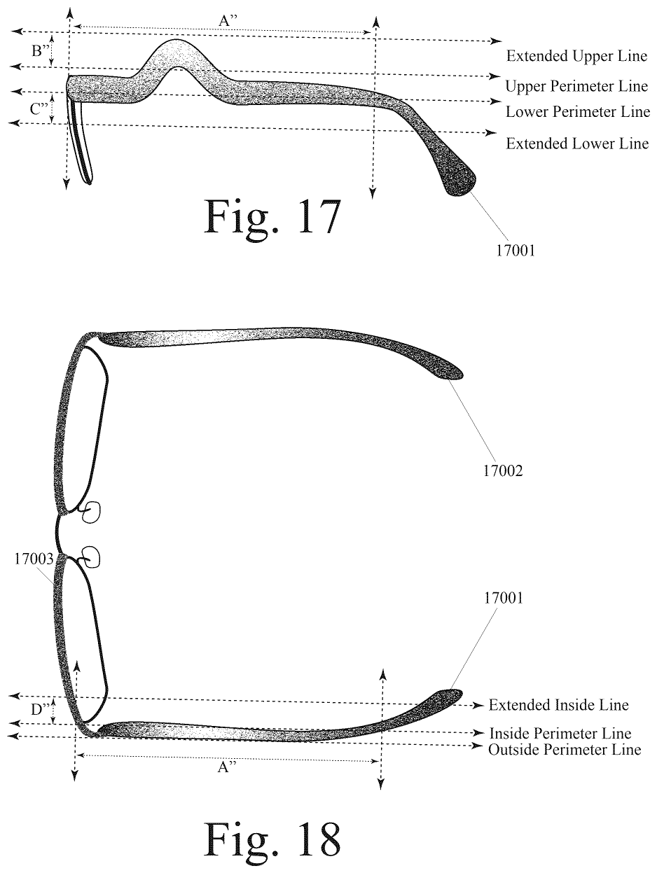

FIGS. 17 and 18 show a side view and a top-down view, respectively, of an eyewear frame with an upward (sinusoidal) wave which comprises a front section 17003 which is configured to span the front of a person's head, a first side section 17001 which is configured to span from a first ear to the front section, and a second side section 17002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, then spans forward (1″-3″) along a relatively-straight longitudinal axis, then curves upward and forward (1″-3″) to a location over the person's temple and/or forehead, and then curves downward and forward to connect to the front section. FIGS. 17 and 18 also show how virtual reference lines can be defined relative to this type of eyewear frame. Metric equivalents can also be used for inch measurements.

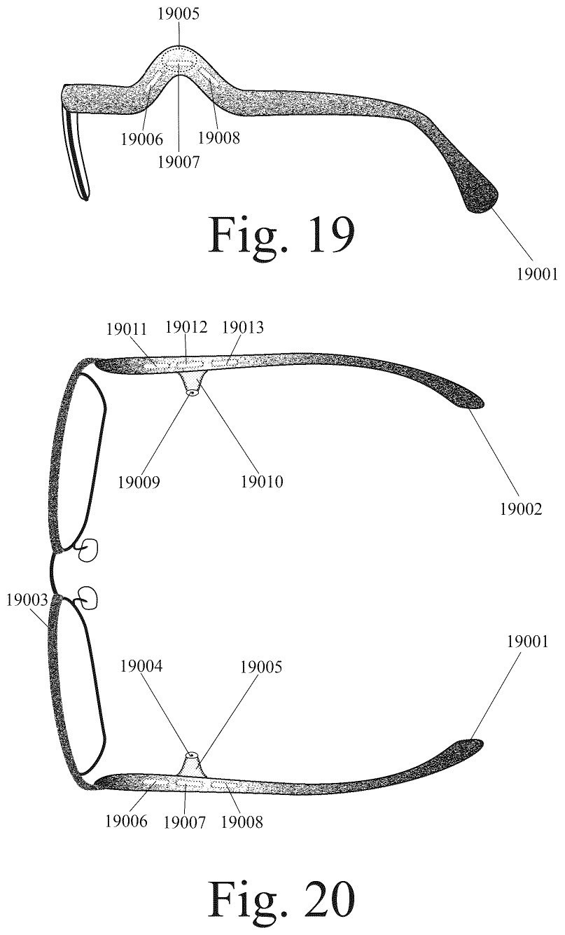

FIGS. 19 and 20 show an example of how this invention can be embodied in electroencephalographic eyewear with the type of upward-wave frame that was shown in FIGS. 17 and 18. FIG. 19 shows a side view. FIG. 20 shows a top-down view. Specifically, FIGS. 19 and 20 show an example of how this invention can be embodied in electroencephalographic eyewear comprising: (a) a frame for eyeglasses or other eyewear; wherein this frame further comprises front section 19003 which is configured to span the front of a person's head, first side section 19001 which is configured to span from a first ear to the front section, and second side section 19002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, then spans forward (1″-3″) along a relatively-straight longitudinal axis, then curves upward and forward (1″-3″) to a location over the person's temple and/or forehead, and then curves downward and forward to connect to the front section; (b) flexible protrusion 19005 which is part of, or attached to, first side section 19001; (c) electromagnetic energy sensor 19004 which collects data concerning electromagnetic brain activity; wherein flexible protrusion 19005 is configured to hold electromagnetic energy sensor 19004 on the person's head; (d) energy source 19006; (e) data processor 19007; and (f) data transmitter and/or receiver 19008.

The example shown in FIGS. 19 and 20 also has a (symmetric) set of components on the other side section, including second flexible protrusion 19010, second electromagnetic energy sensor 19009, second energy source 19011, second data processor 19012, and second data transmitter and/or receiver 19013. In an example, an electromagnetic energy sensor can be located at (or near) the top if the upward wave of the side section. Metric equivalents can also be used for inch measurements. Other relevant components and design variations discussed elsewhere in this disclosure or priority-linked disclosures can also be incorporated into this example.

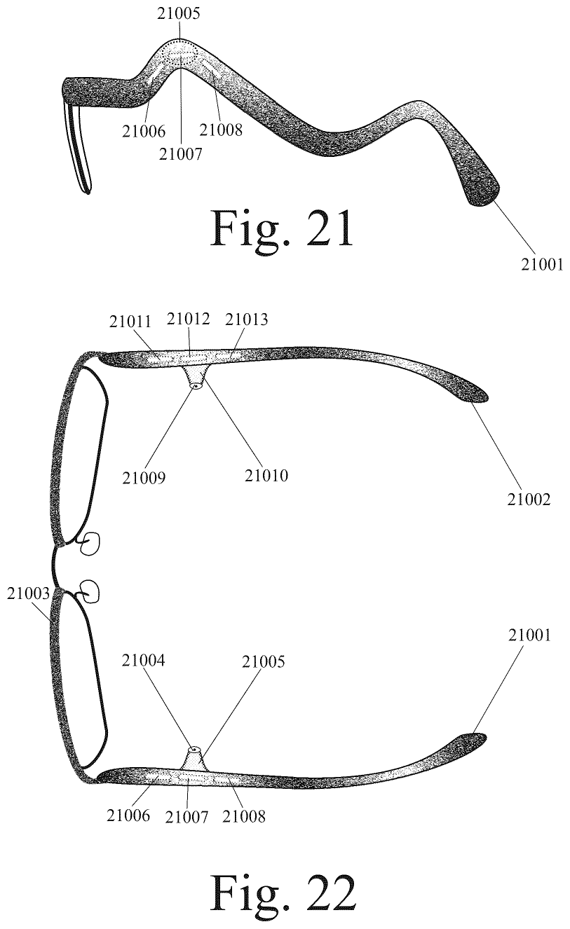

FIGS. 21 and 22 show an example of how this invention can be embodied in electroencephalographic eyewear with a (sinusoidal) undulating frame. FIG. 21 shows a side view. FIG. 22 shows a top-down view. Specifically, FIGS. 21 and 22 show an example of how this invention can be embodied in electroencephalographic eyewear comprising: (a) a frame for eyeglasses or other eyewear; wherein this frame further comprises front section 21003 which is configured to span the front of a person's head, first side section 21001 which is configured to span from a first ear to the front section, and second side section 21002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, then curves downward and forward (1″-3″), then curves upward and forward (1″-3″) to a location over the person's temple and/or forehead, and then curves downward and forward to connect to the front section; (b) flexible protrusion 21005 which is part of, or attached to, selected side section 21001; (c) electromagnetic energy sensor 21004 which collects data concerning electromagnetic brain activity; wherein flexible protrusion 21005 is configured to hold electromagnetic energy sensor 21004 on the person's head; (d) energy source 21006; (e) data processor 21007; and (f) data transmitter and/or receiver 21008.

The example shown in FIGS. 21 and 22 also has a (symmetric) set of components on the other side section, including second flexible protrusion 21010, second electromagnetic energy sensor 21009, second energy source 21011, second data processor 21012, and second data transmitter and/or receiver 21013. Metric equivalents can also be used for inch measurements. Other relevant components and design variations discussed elsewhere in this disclosure or priority-linked disclosures can also be incorporated into this example.

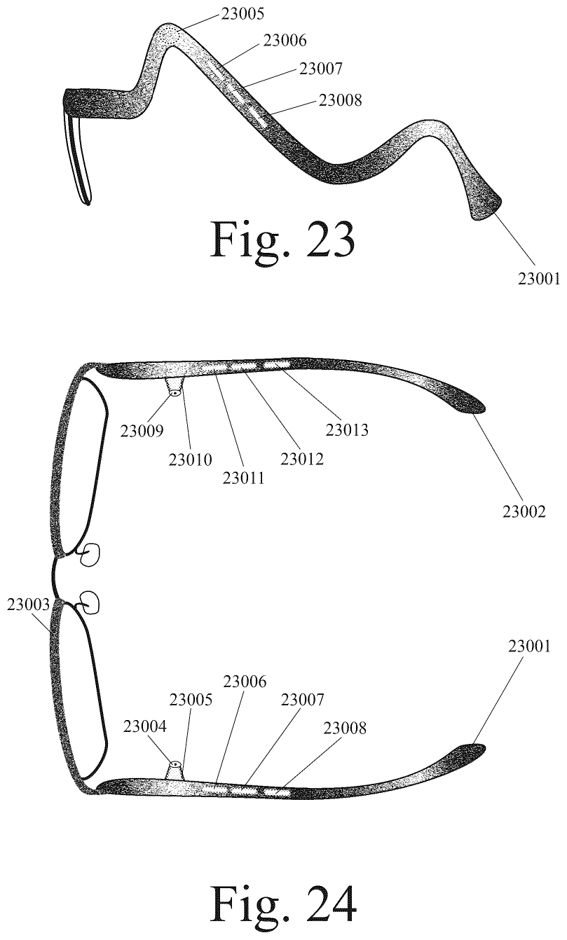

FIGS. 23 and 24 show another example of how this invention can be embodied in electroencephalographic eyewear with a (sinusoidal) undulating frame which is similar to the example shown in FIGS. 21 and 22 except that the energy source, data processor, and data transmitter and/or receiver are in a more-central location. FIG. 23 shows a side view. FIG. 24 shows a top-down view.

Specifically, FIGS. 23 and 24 show an example of how this invention can be embodied in electroencephalographic eyewear comprising: (a) a frame for eyeglasses or other eyewear; wherein this frame further comprises front section 23003 which is configured to span the front of a person's head, first side section 23001 which is configured to span from a first ear to the front section, and second side section 23002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, then curves downward and forward, then curves upward and forward to a location over the person's temple and/or forehead, and then curves downward and forward to connect to the front section; (b) flexible protrusion 23005 which is part of, or attached to, selected side section 23001; (c) electromagnetic energy sensor 23004 which collects data concerning electromagnetic brain activity; wherein flexible protrusion 23005 is configured to hold electromagnetic energy sensor 23004 on the person's head; (d) energy source 23006; (e) data processor 23007; and (f) data transmitter and/or receiver 23008.

The example shown in FIGS. 23 and 24 also has a (symmetric) set of components on the other side section, including second flexible protrusion 23010, second electromagnetic energy sensor 23009, second energy source 23011, second data processor 23012, and second data transmitter and/or receiver 23013. Metric equivalents can also be used for inch measurements. Other relevant components and design variations discussed elsewhere in this disclosure or priority-linked disclosures can also be incorporated into this example.

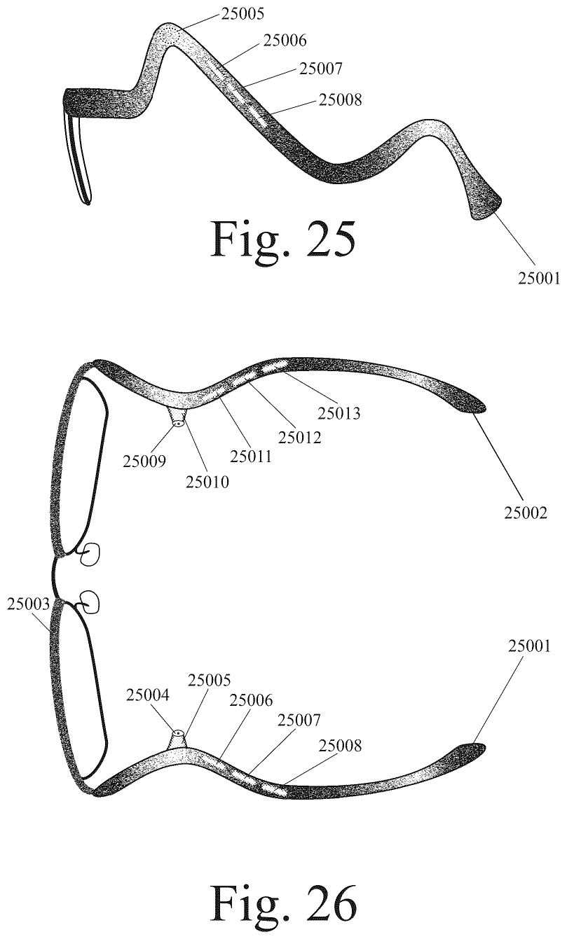

FIGS. 25 and 26 show another example of how this invention can be embodied in electroencephalographic eyewear with a (sinusoidal) undulating frame which is similar to the example shown in FIGS. 23 and 24 except that the anterior (upward wave) portions of the side sections bow inwards toward the person's forehead. In an example, virtual radial lines can be drawn which extend outward into space from the centroid (volume center or mass center) of a person's head. In an example, “outward” can be defined moving farther from this centroid along a virtual radial line and “inward” can be defined as moving closer to this centroid along a virtual radial line. FIG. 25 shows a side view. FIG. 26 shows a top-down view.

Specifically, FIGS. 25 and 26 show an example of how this invention can be embodied in electroencephalographic eyewear comprising: (a) a frame for eyeglasses or other eyewear; wherein this frame further comprises front section 25003 which is configured to span the front of a person's head, first side section 25001 which is configured to span from a first ear to the front section, and second side section 25002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, then curves downward and forward, then curves upward, forward, and inward to a location over the person's temple and/or forehead, and then curves downward, forward, and outward to connect to the front section; (b) flexible protrusion 25005 which is part of, or attached to, selected side section 25001; (c) electromagnetic energy sensor 25004 which collects data concerning electromagnetic brain activity; wherein flexible protrusion 25005 is configured to hold electromagnetic energy sensor 25004 on the person's head; (d) energy source 25006; (e) data processor 25007; and (f) data transmitter and/or receiver 25008.

The example shown in FIGS. 25 and 26 also has a (symmetric) set of components on the other side section, including second flexible protrusion 25010, second electromagnetic energy sensor 25009, second energy source 25011, second data processor 25012, and second data transmitter and/or receiver 25013. Metric equivalents can also be used for inch measurements. Other relevant components and design variations discussed elsewhere in this disclosure or priority-linked disclosures can also be incorporated into this example.

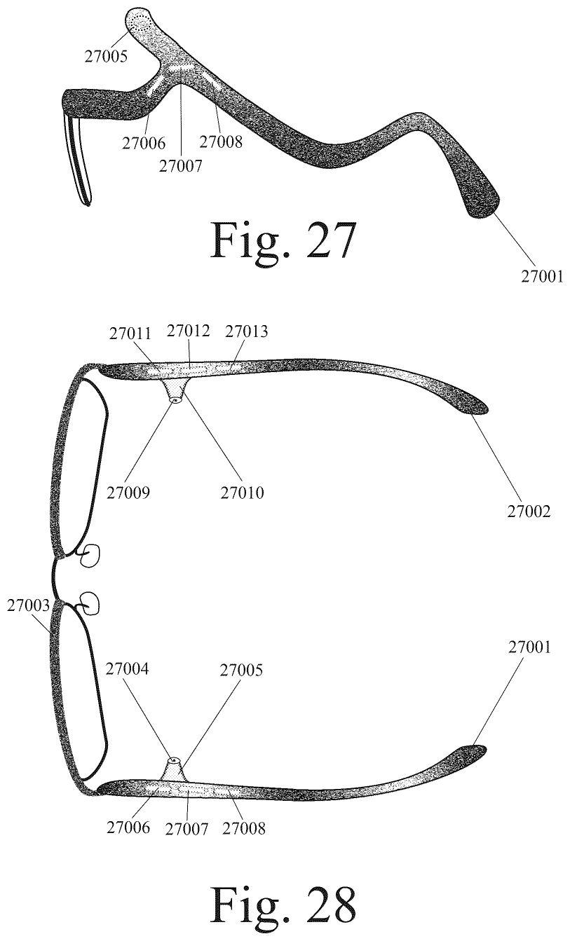

FIGS. 27 and 28 show another example of how this invention can be embodied in electroencephalographic eyewear wherein the side section of the frame has a bifurcation. This bifurcating frame has an upper arm (or projection) which curves up onto the side of the person's forehead. FIG. 27 shows a side view. FIG. 28 shows a top-down view.

Specifically, FIGS. 27 and 28 show an example of how this invention can be embodied in electroencephalographic eyewear comprising: (a) a frame for eyeglasses or other eyewear; wherein this frame further comprises front section 27003 which is configured to span the front of a person's head, first side section 27001 which is configured to span from a first ear to the front section, and second side section 27002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, then curves downward and forward, then curves upward and forward and bifurcates, wherein an upper portion of this bifurcation extends forward (at least ½″) onto the side of the person's forehead and wherein a lower portion of this bifurcation curves forward to connect to the front section; (b) flexible protrusion 27005 which is part of, or attached to, selected side section 27001; (c) electromagnetic energy sensor 27004 which collects data concerning electromagnetic brain activity; wherein flexible protrusion 27005 is configured to hold electromagnetic energy sensor 27004 on the person's head; (d) energy source 27006; (e) data processor 27007; and (f) data transmitter and/or receiver 27008.

The example shown in FIGS. 27 and 28 also has a (symmetric) set of components on the other side section, including second flexible protrusion 27010, second electromagnetic energy sensor 27009, second energy source 27011, second data processor 27012, and second data transmitter and/or receiver 27013. Metric equivalents can also be used for inch measurements. Other relevant components and design variations discussed elsewhere in this disclosure or priority-linked disclosures can also be incorporated into this example.

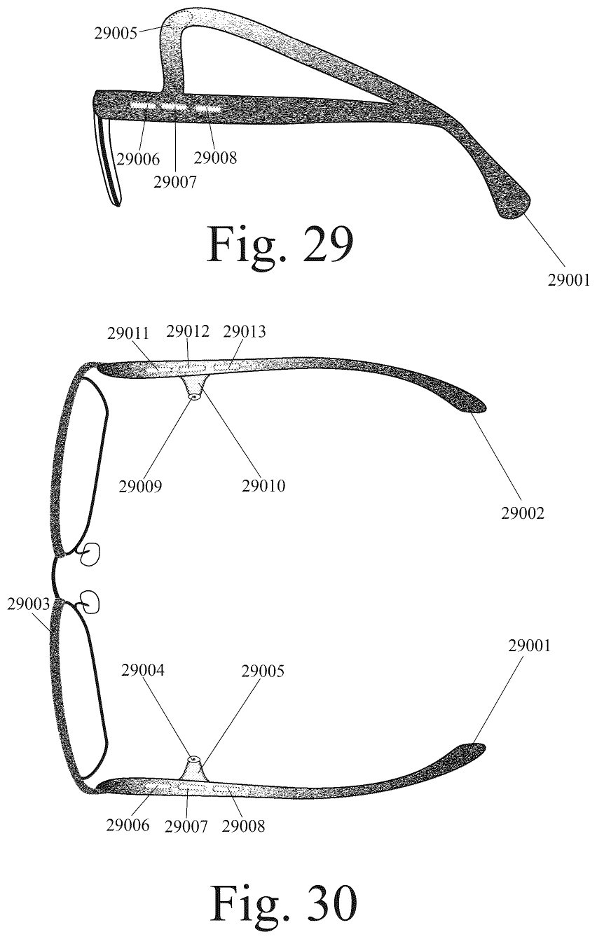

FIGS. 29 and 30 show another example of how this invention can be embodied in electroencephalographic eyewear wherein the side section of the frame has a bifurcation. In this frame, an upper portion of a bifurcation curves up onto the side of the person's forehead and then descends back down to reconnect to the lower portion. FIG. 29 shows a side view. FIG. 30 shows a top-down view.

Specifically, FIGS. 29 and 30 show an example of how this invention can be embodied in electroencephalographic eyewear comprising: (a) a frame for eyeglasses or other eyewear; wherein this frame further comprises front section 29003 which is configured to span the front of a person's head, first side section 29001 which is configured to span from a first ear to the front section, and second side section 29002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, and then bifurcates, wherein a lower portion of this bifurcation spans forward in a relatively-straight axial manner to connect to the front section and wherein an upper portion of this bifurcation curves up onto the side of the person's forehead and then descends to reconnect with the lower portion or front section; (b) flexible protrusion 29005 which is part of, or attached to, selected side section 29001; (c) electromagnetic energy sensor 29004 which collects data concerning electromagnetic brain activity; wherein flexible protrusion 29005 is configured to hold electromagnetic energy sensor 29004 on the person's head; (d) energy source 29006; (e) data processor 29007; and (f) data transmitter and/or receiver 29008.

The example shown in FIGS. 29 and 30 also has a (symmetric) set of components on the other side section, including second flexible protrusion 29010, second electromagnetic energy sensor 29009, second energy source 29011, second data processor 29012, and second data transmitter and/or receiver 29013. Metric equivalents can also be used for inch measurements. Other relevant components and design variations discussed elsewhere in this disclosure or priority-linked disclosures can also be incorporated into this example.

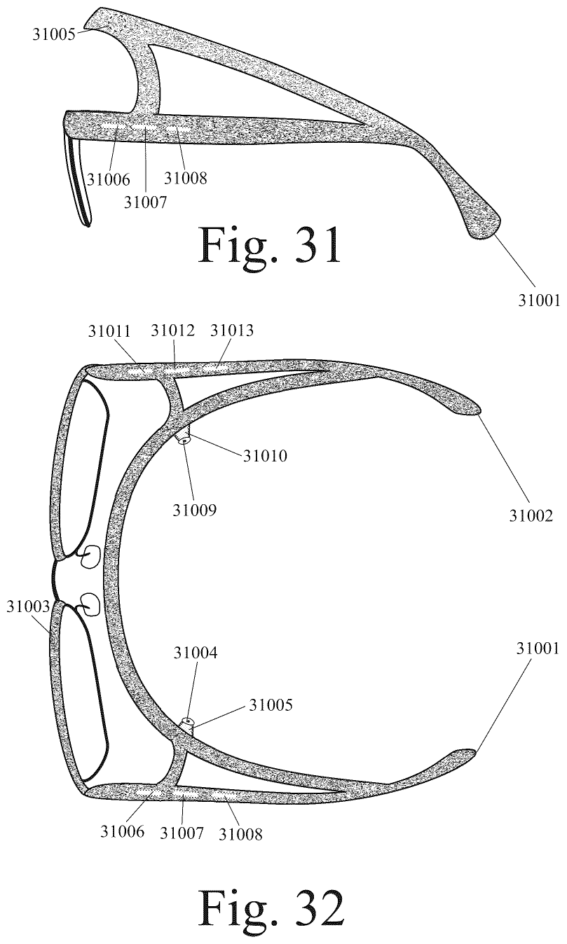

FIGS. 31 and 32 show an example of how this invention can be embodied in electroencephalographic eyewear that includes an upper (second) front section which curves entirely around a person's forehead. FIG. 31 shows a side view. FIG. 32 shows a top-down view.

Specifically, FIGS. 31 and 32 show an example of how this invention can be embodied in electroencephalographic eyewear comprising: (a) a frame for eyeglasses or other eyewear; wherein this frame further comprises front section 31003 which is configured to span the front of a person's head, first side section 31001 which is configured to span from a first ear to the front section, and second side section 31002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, and then bifurcates, wherein an upper portion of this bifurcation curves across the person's forehead above the front section and wherein a lower portion of this bifurcation spans forward in a relatively-straight axial manner to connect to the front section; (b) flexible protrusion 31005 which is part of, or attached to, selected side section 31001; (c) electromagnetic energy sensor 31004 which collects data concerning electromagnetic brain activity; wherein flexible protrusion 31005 is configured to hold electromagnetic energy sensor 31004 on the person's head; (d) energy source 31006; (e) data processor 31007; and (f) data transmitter and/or receiver 31008.

The example shown in FIGS. 31 and 32 also has a (symmetric) set of components on the other side section, including second flexible protrusion 31010, second electromagnetic energy sensor 31009, second energy source 31011, second data processor 31012, and second data transmitter and/or receiver 31013. In this example, there is also an anterior connecting strut (or connector) between the upper portion of the bifurcation and the lower portion of the bifurcation. In this example, this anterior connecting strut forms one side of a triangular gap, wherein the other two sides of this triangular gap are formed by the upper and lower portions of the bifurcation. In an example, the upper portion of the bifurcation can be transparent. In an example, the upper portion of the bifurcation can be made from fabric. In an example, the upper portion of the bifurcation can be elastic and/or stretchable. Metric equivalents can also be used for inch measurements. Other relevant components and design variations discussed elsewhere in this disclosure or priority-linked disclosures can also be incorporated into this example.

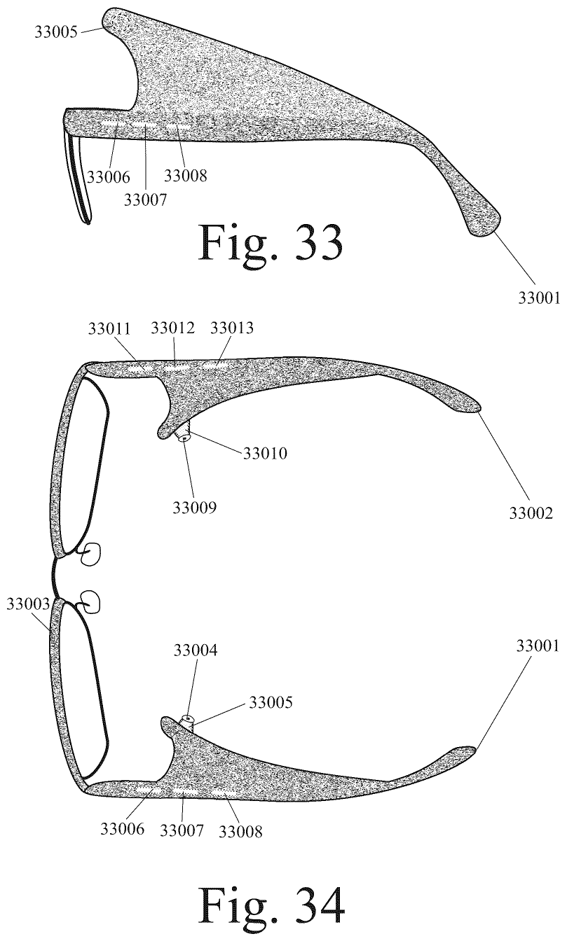

FIGS. 33 and 34 show an example of how this invention can be embodied in electroencephalographic eyewear with a fin or wedge shaped side portion which curves upward and inward onto the side of a person's forehead. FIG. 33 shows a side view. FIG. 34 shows a top-down view.

Specifically, FIGS. 33 and 34 show an example of how this invention can be embodied in electroencephalographic eyewear comprising: (a) a frame for eyeglasses or other eyewear; wherein this frame further comprises front section 33003 which is configured to span the front of a person's head, first side section 33001 which is configured to span from a first ear to the front section, and second side section 33002 which is configured to span from a second ear to the front section; wherein the first side section starts with a posterior end which is configured to be worn posterior to a person's ear, then curves upward and forward around the tissue connection between the person's outer ear to the rest of the person's head to the top of this tissue connection, and then widens (fans out, broadens, or expands) into a fin (wedge or triangular) shaped structure which is configured to curve upward and inward onto the side of the person's forehead and also connect to the front section; (b) flexible protrusion 33005 which is part of, or attached to, selected side section 33001; (c) electromagnetic energy sensor 33004 which collects data concerning electromagnetic brain activity; wherein flexible protrusion 33005 is configured to hold electromagnetic energy sensor 33004 on the person's head; (d) energy source 33006; (e) data processor 33007; and (f) data transmitter and/or receiver 33008.

The example shown in FIGS. 33 and 34 also has a (symmetric) set of components on the other side section, including second flexible protrusion 33010, second electromagnetic energy sensor 33009, second energy source 33011, second data processor 33012, and second data transmitter and/or receiver 33013. In an example, the fin (wedge or triangular) shaped structure in this example can be soft and compressible. In an example, the fin (wedge or triangular) shaped structure in this example can be made from compressible foam or be an inflatable member (such as a balloon). In an example, the fin (wedge or triangular) shaped structure in this example can be made from plastic or metal.

In an example, the fin (wedge or triangular) shaped structure can widen from a posterior portion width of less than ½″ to an anterior width of greater than ½″. In an example, the fin (wedge or triangular) shaped structure can widen from a posterior portion width of less than ½″ to an anterior width of greater than ¾″. Metric equivalents can also be used for inch measurements. Other relevant components and design variations discussed elsewhere in this disclosure or priority-linked disclosures can also be incorporated into this example.

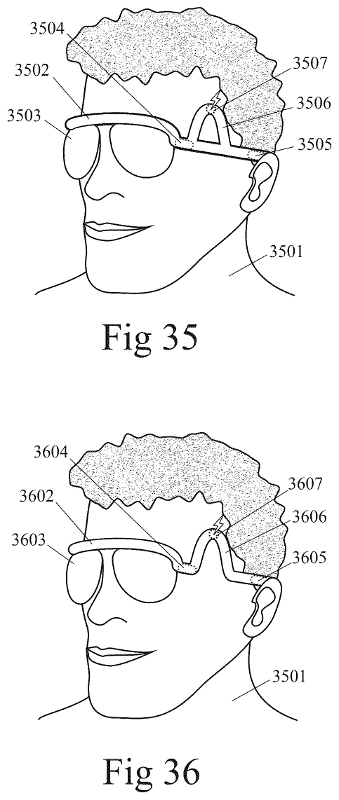

FIG. 35 shows an example of eyewear for monitoring a person's electromagnetic brain activity comprising: at least one optical member which is configured to be held in proximity to an eye; a support member with at least one upward protrusion which is configured to span a portion of a person's forehead, temple, and/or a side of the person's head; and at least one electromagnetic brain activity sensor which is held in place by the upward protrusion. The example in FIG. 35 further comprises at least one imaging member and a data processing unit.

Specifically, FIG. 35 shows an example of eyewear for monitoring a person's (3501) electromagnetic brain activity comprising: at least one optical member (3503) which is configured to be held in proximity to an eye; a support member (3502) with at least one upward protrusion (3506) which is configured to span a portion of a person's forehead, temple, and/or a side of the person's head; and at least one electromagnetic brain activity sensor (3507) which is held in place by upward protrusion (3506). The example in FIG. 35 further comprises at least one imaging member (3504) and a data processing unit (3505).

In FIG. 35, upward protrusion 3506 ascends from a side portion of support member 3502. In this example, upward protrusion 3506 has a sinusoidal section shape. In an example, an upward protrusion can have a conic section shape. In this example, upward protrusion 3506 is one of two support member pathways which span from a person's ear to the front of the person's face. In this example, the other support member pathway is relatively straight. In this example, an electromagnetic energy sensor measures the conductivity, voltage, impedance, or resistance of electromagnetic energy transmitted through body tissue. In this example, electromagnetic brain activity sensor 3507 is an EEG sensor which is held in place by upward protrusion 3506. This example can include other component variations which were discussed earlier.

FIG. 36 shows an example of eyewear for monitoring a person's electromagnetic brain activity comprising: at least one optical member which is configured to be held in proximity to an eye; a support member with at least one upward protrusion which is configured to span a portion of a person's forehead, temple, and/or a side of the person's head; and at least one electromagnetic brain activity sensor which is held in place by the upward protrusion. The example in FIG. 36 further comprises at least one imaging member and a data processing unit.

Specifically, FIG. 36 shows an example of eyewear for monitoring a person's (3601) electromagnetic brain activity comprising: at least one optical member (3603) which is configured to be held in proximity to an eye; a support member (3602) with at least one upward protrusion (3606) which is configured to span a portion of a person's forehead, temple, and/or a side of the person's head; and at least one electromagnetic brain activity sensor (3607) which is held in place by upward protrusion (3606). The example in FIG. 36 further comprises at least one imaging member (3604) and a data processing unit (3605).

In FIG. 36, upward protrusion 3606 ascends from a side portion of support member 3602. In this example, upward protrusion 3606 has a sinusoidal section shape. In an example, an upward protrusion can have a conic section shape. In this example, upward protrusion 3606 is the sole pathway which spans from a person's ear to the front of the person's face. In this example, an electromagnetic energy sensor measures the conductivity, voltage, impedance, or resistance of electromagnetic energy transmitted through body tissue. In this example, electromagnetic brain activity sensor 3607 is an EEG sensor which is held in place by upward protrusion 3606. This example can include other component variations which were discussed earlier.

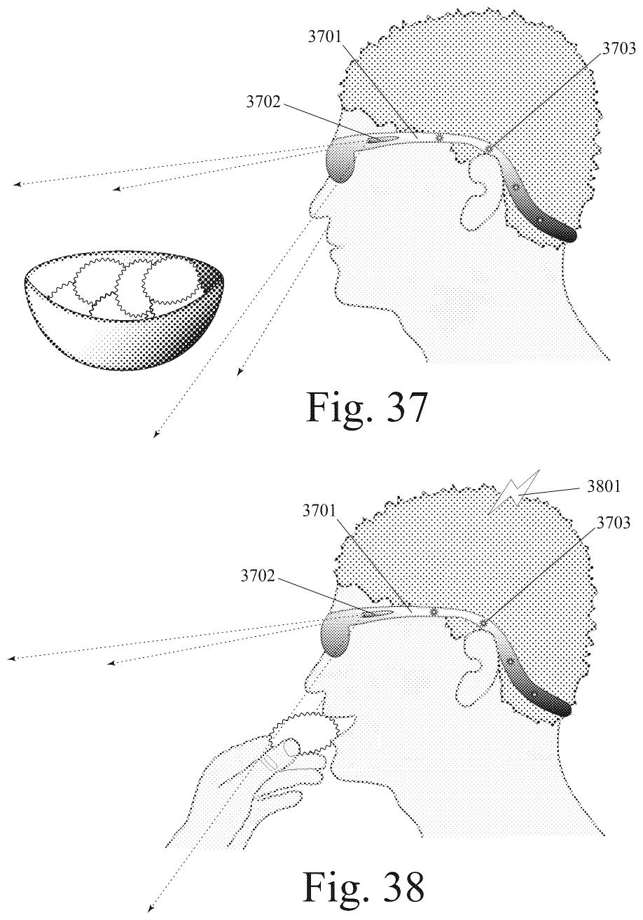

FIGS. 37 and 38 show an example of a device that doubles as eyewear and can be used to measure and/or modify a person's food consumption. In this example, wearable EEG monitor 3701 comprises a plurality of electrodes or other brain activity sensors (including 3703) and two wearable cameras (including 3702 shown on the left side). In this example, this device is assumed to be left-right symmetric, so a second camera is assumed to be on the right side of the person's head. In an example, wearable EEG monitor 3701 can further comprise a control unit. In an example, this control unit can comprise a power source, data processor, and data transmitter.

As shown in FIGS. 37 and 38, the anterior portion of wearable EEG monitor 3701 comprises an eyewear frame. In this example, this eyewear frame includes lenses. In an example, this eyewear frame can include a display surface instead of lenses. In an example, lenses can function as a display surface. In an example, this eyewear frame can be rigid, semi-rigid, or flexible.

As shown in FIGS. 37 and 38, the posterior portion of wearable EEG monitor 3701 comprises an arcuate member which loops around the lower-rear portion of the back of the person's head at a level which is equal to, or lower than, the person's ears. The sides of this device rest on top of the person's ears. In an example, this posterior arcuate portion of this device can have the same degree of rigidity, flexibility, and/or elasticity as the anterior eyewear frame portion of this device. In an example, this posterior arcuate portion of this device can have a higher degree of flexibility and/or elasticity than the anterior eyewear frame portion of this device. In an example, the anterior eyewear frame portion of this device can be made of metal and/or plastic and the posterior arcuate portion of this device can be made of fabric.

As shown in FIGS. 37 and 38, the two wearable cameras (including 3702 on the left side) of this device can take stereoscopic pictures of food when the person is looking at food (see FIG. 37) and when the person is eating food (see FIG. 38). In an example, having images of food both before and during consumption can enable more accurate identification of food type and more accurate measure of food quantity consumed. Also, stereoscopic imaging of food can enable 3D and volumetric modeling to better estimate the quantity of food consumed.

FIG. 38 shows a change 3801 in electromagnetic brain activity that is triggered when the person eats food. This change 3801 in electromagnetic brain activity is measured by wearable EEG monitor 3701. This change 3801 in brain activity based on food consumption is then linked to previously-identified patterns of food consumption and used to estimate the type and quantity of food consumed.

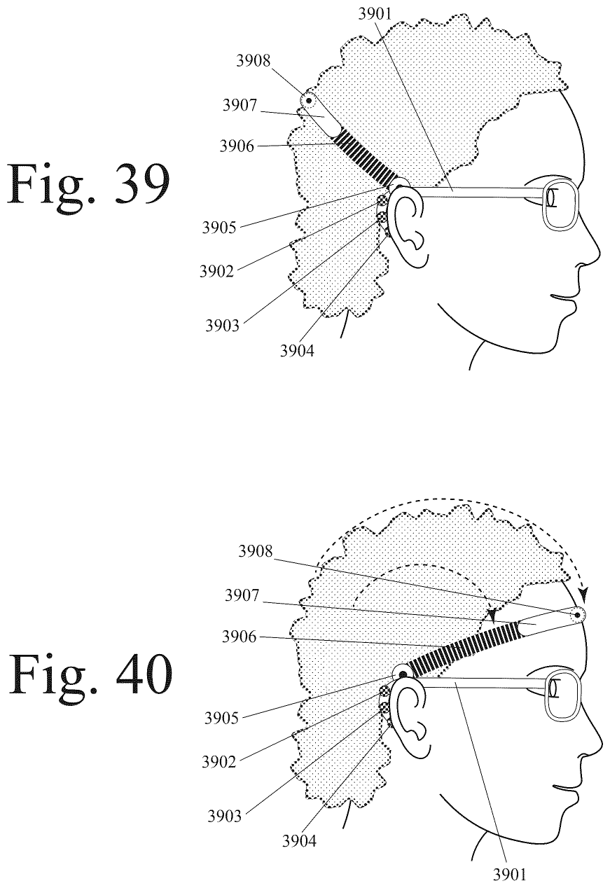

FIGS. 39 and 40 show two sequential views of a wearable device for measuring electromagnetic brain activity. FIG. 39 shows a view of this example at a first time wherein a movable loop with one or more electromagnetic energy sensors is configured to loop around the rear and/or upper-rear portion of a person's head. FIG. 40 shows a view of this example at a second time wherein the movable loop has been moved so that it is configured to loop around the person's forehead.

FIGS. 39 and 40 show an example of a wearable device for measuring electromagnetic brain activity comprising: eyewear 3901; a movable loop (including joint 3905, stretchable portion 3906, and end portion 3907), wherein this movable loop has a first configuration in which it loops around the rear and/or upper-rear portion of a person's head, wherein this movable loop has a second configuration in which is loops across the person's forehead, and wherein this movable loop can be reversibly moved from the first configuration to the second configuration; at least one electromagnetic energy sensor 3908 which is configured to be held in proximity to the person's forehead by the movable loop in the second configuration, wherein the electromagnetic energy sensor collects data concerning electromagnetic activity of the person's brain; a wireless data transmitter and/or receiver 3902; a data processor 3903; and a power source 3904. In an example, this device can have a symmetric configuration on the other side of the person's head, which is not shown here.

In an example, a movable loop can include a joint, hinge, or axle. In an example, a movable loop can pivot or rotate around a joint, hinge, or axle. In an example, the portion of a movable loop which is furthest from a person's ear can pivot or rotate around a joint, hinge, or axle which is within 1″ of a person's ear. In an example, the portion of a movable loop which is furthest from a person's ear can pivot or rotate around a joint, hinge, or axle which is within 3″ of a person's ear. In an example, a movable loop can be manually and reversibly moved from its first configuration to its second configuration. In an example, a joint, hinge, or axle can be reversibly locked or unlocked, so as to reversibly lock a movable loop in its first configuration or second configuration.

In an example, a movable loop can have a first configuration in which it loops around the rear and/or upper-rear portion of a person's head and a second configuration in which it loops around (across) a person's forehead. In an example, a movable loop can transition from its first configuration to its second configuration by pivoting or rotating around a joint, hinge, or axle. In an example, a movable loop can have a first configuration wherein its longitudinal axis is parallel to a vector between the 9 o'clock (270 degree) and 11 o'clock (330 degree) vectors and can have a second configuration wherein its longitudinal axis is parallel to a vector between the 1 o'clock (30 degree) and 3 o'clock (90 degree) vectors. In an example, a movable loop can have a first configuration wherein its longitudinal axis is parallel to a vector between the 10 o'clock (300 degree) and 12 o'clock (0 degree) vectors and can have a second configuration wherein its longitudinal axis is parallel to a vector between the 1 o'clock (30 degree) and 3 o'clock (90 degree) vectors.

In an example, a movable loop can be stretchable, elastic, and/or expandable. In an example, a movable loop can further comprise a first portion with a first degree of stretchability, elasticity, and/or expandability and a second portion with a second degree of stretchability, elasticity, and/or expandability, wherein the second degree is less than the first degree. In the example shown in FIGS. 39 and 40, the movable loop has a stretchable portion 3906 (with a greater degree of stretchability) and an end portion 3907 (with a lower degree of stretchability). Having at least one stretchable, elastic, and/or expandable portion of a movable loop allows the loop to be more easily moved from its first configuration to its second configuration. Having at least one stretchable, elastic, and/or expandable portion of a movable loop can also enable to loop to hold one or more electromagnetic energy sensors more securely against a person's forehead in the second configuration.

In an example, the stretchable portion of a movable loop can be an elastic band or strap. In an example, the stretchable portion of a movable loop can include a spring mechanism. In an example, a movable loop can include telescoping members. In an example, telescoping members can be held in tension by a spring mechanism so that they are compelled toward a contracted configuration in order to fit snugly against a person's head. In an example, a movable loop can have a first perimeter distance in a first configuration and a second perimeter distance in a second configuration, wherein the first distance is shorter than the second distance.

In an example, a movable loop and eyeglasses (or other eyewear) can be integral components of a single wearable device. In an example, a movable loop can be a separate device which is attached to eyeglasses (or other eyewear). In an example, a movable loop can be configured to receive the side frame of a pair of eyeglasses (or other eyewear). In an example, a movable loop can further comprise an opening which is configured to receive the side-piece of an eyeglass (or other eyewear) frame. In an example, a movable loop can further comprise a clip or other attachment mechanism to which the side-piece of an eyeglass (or other eyewear) frame can be attached. Other relevant variations and components discussed in other portions of this concurrent disclosure or prior disclosures incorporated herein by reference can also be applied to this example.

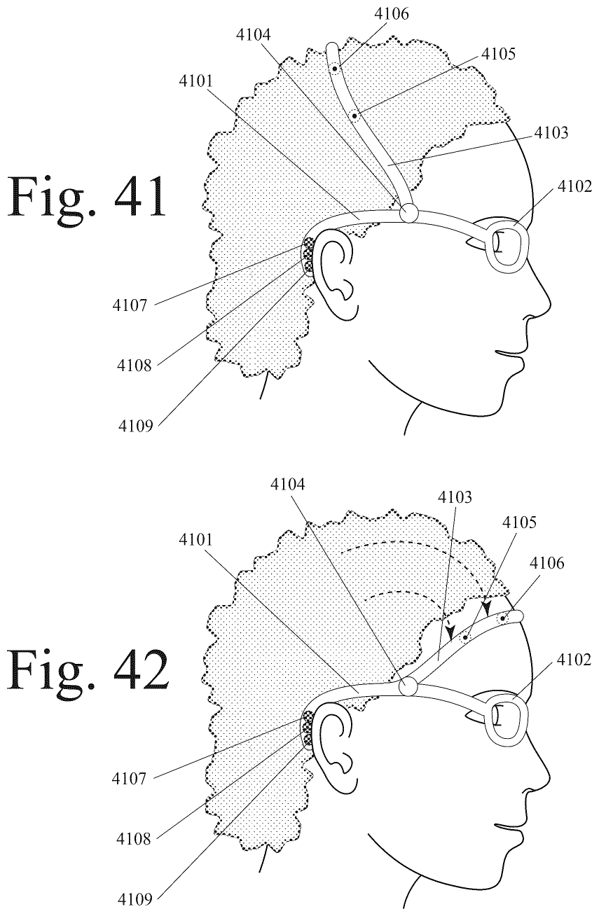

FIGS. 41 and 42 show two sequential views of a wearable and mobile Brain Computer Interface (BCI) comprising: eyewear which further comprises—a side frame (4101), a front frame (4102), a joint (4104) on the side frame, a movable loop (4103) which is configured to loop over the top of a person's head or around back of a person's head in a first configuration and which is configured to span across a person's forehead in a second configuration, wherein the movable loop pivots and/or rotates around the joint from the first configuration to the second configuration; one or more electromagnetic energy sensors (4105 and 4106) which are part of, or attached to, the movable loop, wherein these electromagnetic energy sensors collect data concerning electromagnetic brain activity; a power source (4107); a data processor (4108); and a data transmitter and/or receiver (4109). FIG. 41 shows this device when the movable loop is in the first configuration. FIG. 42 shows this device when the movable loop is in the second configuration. In an example, this device can be symmetric, with symmetric components and structure on the other side of the person's head.

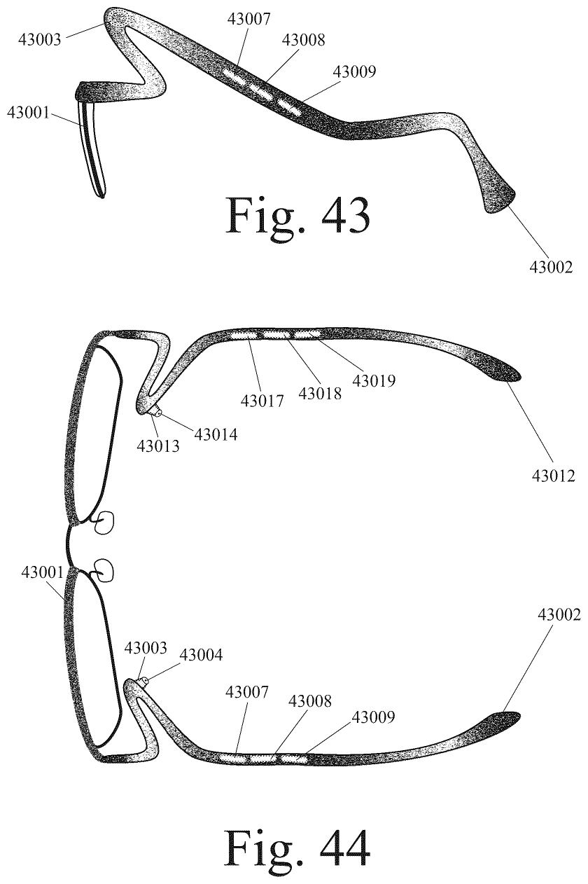

FIGS. 43 and 44 show side and top-down views, respectively, of an example of EEG glasses (electroencephalographic eyewear) comprising: (a) a front section of an eyewear frame which is configured to span the front of a person's face; (b) a side section of the eyewear frame which is configured to: span forward from one of the person's ears; then span upward, forward, and inward (toward the center of the person's forehead) to a location over the person's forehead (above one of the person's eyes); then span downward, backward, and outward (away from the center of the person's forehead); and then span forward to connect to the front section; and (c) at least one electromagnetic energy sensor which collects data concerning electromagnetic brain activity which is attached to the side section. In an example, the eyewear from can include a second side section with a similar configuration.

This example of EEG glasses (electroencephalographic eyewear) can also be described as comprising: (a) a front section of an eyewear frame which is configured to span the front of a person's face; (b) a side section of the eyewear frame which includes an upward loop which is configured to curve upward, forward, and inward (toward the center of the person's forehead) to a location over the person's forehead (above one of the person's eyes) and then curve downward, backward, and outward (away from the center of the person's forehead); and (c) at least one electromagnetic energy sensor which collects data concerning electromagnetic brain activity which is attached to the side section. In an example, the eyewear from can include a second side section with a similar configuration.

This example of EEG glasses (electroencephalographic eyewear) can also be described as comprising: (a) a front section of an eyewear frame which is configured to span the front of a person's face; (b) a side section of the eyewear frame with an arcuate zigzag portion, wherein this arcuate zigzag portion is configured to span forward (to a location over the person's forehead above one of the person's eyes), then span backward (to a location over the person's temple), and then span forward again (to connect to the front section); and (c) at least one electromagnetic energy sensor which collects data concerning electromagnetic brain activity which is attached to the side section. In an example, the eyewear from can include a second side section with a similar configuration.

This example of EEG glasses (electroencephalographic eyewear) can also be described as comprising: (a) a front section of an eyewear frame which is configured to span the front of a person's face; (b) a side section of the eyewear frame with an arcuate zigzag portion, wherein this arcuate zigzag portion is configured to span forward and upward (to a location over the person's forehead above one of the person's eyes), then span backward and downward, and then span forward (to connect to the front section); and (c) at least one electromagnetic energy sensor which collects data concerning electromagnetic brain activity which is attached to the side section. In an example, the eyewear from can include a second side section with a similar configuration.

This example of EEG glasses (electroencephalographic eyewear) can also be described as comprising: (a) a front section of an eyewear frame which is configured to span the front of a person's face; (b) a side section of the eyewear frame with an arcuate zigzag portion, wherein this arcuate zigzag portion is configured to span forward, upward, and inward toward the center of the person's forehead (to a location over the person's forehead above one of the person's eyes), then span backward, downward, and outward away from the center of the person's forehead, and then span forward (to connect to the front section); and (c) at least one electromagnetic energy sensor which collects data concerning electromagnetic brain activity which is attached to the side section. In an example, the eyewear from can include a second side section with a similar configuration.

With respect to specific components, FIGS. 43 and 44 show side and top-down views, respectively, of an example of EEG glasses (electroencephalographic eyewear) comprising: a front section 43001 of an eyewear frame; a first side section 43002 of the eyewear frame, wherein the first side section spans from one of the person's ears to the front section of the eyewear frame, wherein the first side section includes a loop which curves upward, forward, and inward (closer to the center of the person's forehead) to a location over the person's forehead (above an eye) and then curves downward, backward, and outward (farther from the center of the person's forehead); a first flexible protrusion 43003 on the first side section; a first electromagnetic energy sensor 43004 which collects data concerning electromagnetic brain activity on the first flexible protrusion; a first energy source 43007; a first data processor 43008; a first data transmitter and/or receiver 43009; a second side section 43012 of the eyewear frame, wherein the second side section spans from one of the person's ears to the front section of the eyewear frame, wherein the second side section includes a loop which curves upward, forward, and inward (closer to the center of the person's forehead) to a location over the person's forehead (above an eye) and then curves downward, backward, and outward (farther from the center of the person's forehead); a second flexible protrusion 43013 on the second side section; a second electromagnetic energy sensor 43014 which collects data concerning electromagnetic brain activity on the second flexible protrusion; a second energy source 43017; a second data processor 43018; and a second data transmitter and/or receiver 43019.

In this example, there is an energy source, a data processor, and a data transmitter and/or receiver on each side section. In an example, there can be an energy source, data processor, and data transmitter and/or receiver on only one side section. In an example, a flexible protrusion and/or an electromagnetic energy sensor can be attached to the portion of a side section which is located over the person's forehead above an eye. In this example, there is only one electromagnetic energy sensor on a side section. In an example, there can be two or more electromagnetic energy sensors on a side section. Other relevant components and design variations discussed elsewhere in this disclosure or priority-linked disclosures can also be incorporated into this example.

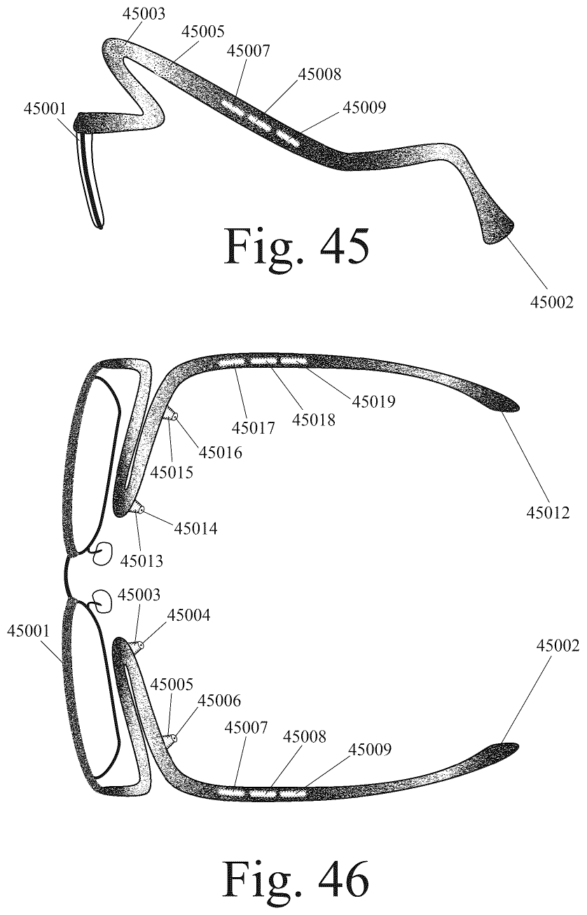

FIGS. 45 and 46 show an example of EEG glasses (electroencephalographic eyewear) which is similar to the example shown in FIGS. 43 and 44 except that: the side sections extend further inward toward the center of the person's forehead; and there are two electromagnetic energy sensors on each side section. In an example, inward loops of right-side and left-side sections may be separated by a distance of 5″ or less (across a person's forehead). In an example, inward loops of right-side and left-side sections may be separated by a distance of 3″ or less (across a person's forehead).

With respect to specific components, FIGS. 45 and 46 show side and top-down views, respectively, of an example of EEG glasses (electroencephalographic eyewear) comprising: a front section 45001 of an eyewear frame; a first side section 45002 of the eyewear frame, wherein the first side section spans from one of the person's ears to the front section of the eyewear frame, wherein the first side section includes a loop which curves upward, forward, and inward (closer to the center of the person's forehead) to a location over the person's forehead (above an eye) and then curves downward, backward, and outward (farther from the center of the person's forehead); first and second flexible protrusions 45003 and 45005 on the first side section; first and second electromagnetic energy sensors 45004 and 45006 which collect data concerning electromagnetic brain activity on the first flexible protrusion; a first energy source 45007; a first data processor 45008; a first data transmitter and/or receiver 45009; a second side section 45012 of the eyewear frame, wherein the second side section spans from one of the person's ears to the front section of the eyewear frame, wherein the second side section includes a loop which curves upward, forward, and inward (closer to the center of the person's forehead) to a location over the person's forehead (above an eye) and then curves downward, backward, and outward (farther from the center of the person's forehead); third and fourth flexible protrusions 45013 and 45015 on the second side section; third and fourth electromagnetic energy sensors 45014 and 45016 which collect data concerning electromagnetic brain activity on the second flexible protrusion; a second energy source 45017; a second data processor 45018; and a second data transmitter and/or receiver 45019. Other relevant components and design variations discussed elsewhere in this disclosure or priority-linked disclosures can also be incorporated into this example.

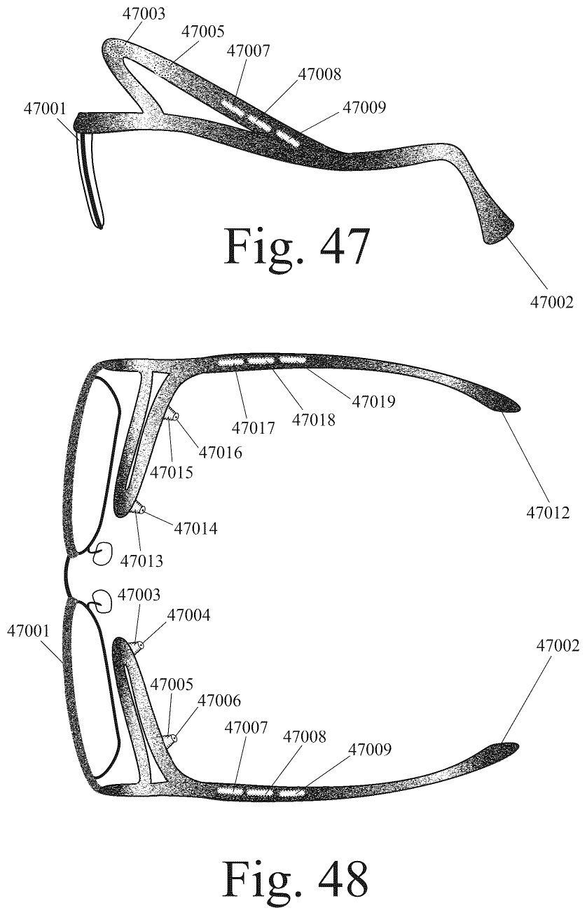

FIGS. 47 and 48 show an example of EEG glasses (electroencephalographic eyewear) which is similar to the example shown in FIGS. 45 and 46 except that the side sections each include a direct link which connects the ends of a forward-upward loop. This creates a bifurcation in a side section: with a first branch of the side section extending forward, upward, and inward to a location over the person's forehead; and a section branch of the side section extending in a relatively straight manner from the person's ear to the front sector of the eyewear frame.

With respect to specific components, FIGS. 47 and 48 show side and top-down views, respectively, of an example of EEG glasses (electroencephalographic eyewear) comprising: a front section 47001 of an eyewear frame; a first side section 47002 of the eyewear frame, wherein the first side section spans from one of the person's ears to the front section of the eyewear frame, wherein the first side section includes a loop which curves upward, forward, and inward (closer to the center of the person's forehead) to a location over the person's forehead (above an eye) and then curves downward, backward, and outward (farther from the center of the person's forehead); first and second flexible protrusions 47003 and 47005 on the first side section; first and second electromagnetic energy sensors 47004 and 47006 which collect data concerning electromagnetic brain activity on the first flexible protrusion; a first energy source 47007; a first data processor 47008; a first data transmitter and/or receiver 47009; a second side section 47012 of the eyewear frame, wherein the second side section spans from one of the person's ears to the front section of the eyewear frame, wherein the second side section includes a loop which curves upward, forward, and inward (closer to the center of the person's forehead) to a location over the person's forehead (above an eye) and then curves downward, backward, and outward (farther from the center of the person's forehead); third and fourth flexible protrusions 47013 and 47015 on the second side section; third and fourth electromagnetic energy sensors 47014 and 47016 which collect data concerning electromagnetic brain activity on the second flexible protrusion; a second energy source 47017; a second data processor 47018; and a second data transmitter and/or receiver 47019. Other relevant components and design variations discussed elsewhere in this disclosure or priority-linked disclosures can also be incorporated into this example.

In an example, the side frame of eyewear can be configured to span from a person's ear to a front frame. In an example, a rear portion of a side frame can curve around the rear of the person's outer ear. In an example, a side frame can be arcuate. In an example, a portion of a side frame between a person's ear and a front frame can arc, curve, wave, and/or undulate upwards. In an example, a side frame can have a downward-facing concave portion. In an example, a front frame of eyewear can hold one or more lenses. In an example, this eyewear can be a pair of eyeglasses. In an example, the front frame of eyewear can hold one or more image displays. In an example, this eyewear can be virtual reality (VR) and/or augmented reality (AR) eyewear.

In an example, the joint around which a movable loop pivots and/or rotates can be located along the (rear to front) longitudinal mid-section of a side frame. In an example, a joint can be located within 2″ of the longitudinal mid-point of a side frame. In an example, a joint around which a movable loop pivots and/or rotates can be located along the rear third of a side frame. In an example, a joint can be located within 2″ of the rear end a side frame. In an example, a joint can further comprise a locking mechanism which locks it in place when a movable loop is at a selected angle and/or in a selected position. In an example, a joint can have restricted movement such that it restricts the movement of a movable loop so that the loop does not descend lower than a selected position on a person's forehead.

In an example, a movable loop can be made out of metal or a polymer. In an example, a movable loop can be flexibly resilient. In an example, a movable loop can be made out of fabric. In an example, a movable loop can be elastic, stretchable, and/or expandable. In an example, a movable loop can further comprise an elastic, stretchable, and/or expandable portion. In an example, a movable loop can further comprise a telescoping portion. In an example, a movable loop holds one or more electromagnetic energy sensors on a person's forehead when the loop is in the second configuration. Other relevant variations and components discussed in other portions of this concurrent disclosure or prior disclosures incorporated herein by reference can also be applied to this example.

In an example, an electromagnetic energy sensor for collecting data concerning electromagnetic brain activity can be an electroencephalographic (EEG) sensor. In an example, an electromagnetic energy sensor can be an electrode. In an example, an electromagnetic energy sensor can be a dry electrode. In an example, there can be two or more electromagnetic energy sensors which collect data concerning electromagnetic brain activity.

In an example, one or more electromagnetic energy sensors can be modular. In an example, one or more electromagnetic energy sensors can be removably attached. In an example, a device can comprise a first number of electromagnetic energy sensors and a second number of locations where electromagnetic energy sensors can be attached, wherein the second number is greater than the first number. In an example, one or more electromagnetic energy sensors can be removably attached by one or more attachment mechanisms selected from the group consisting of: magnetic attachment; hook-and-eye fabric; protrusion and opening; snap; clip; clasp; hook; buckle; plug attachment; pin; button; thread and groove; tongue and groove.

In an example, data concerning a person's brain activity can be collected by one or more electromagnetic energy sensors at one or multiple selected recording sites. In an example, the locations of one or more electromagnetic energy sensors can be selected from the group of EEG placement sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, one or more reference places can be selected from the group of sites consisting of A1 and A2.

In an example, collection of data concerning brain activity can comprise measuring electromagnetic data concerning impedance, voltage difference, and/or energy transfer between two sites on a person's head—a selected recording site and a reference site. In an example, electromagnetic brain activity data can be collected by an electromagnetic energy sensor at a selected recording place. In an example, electromagnetic brain activity data from a selected recording place (relative to a reference place) can be called a “channel.” In an example, electromagnetic brain activity data from multiple recording places can be called a “montage.” In an example, brain activity data can be recorded at a rate in the range of 100 to 300 samples per second.

In an example, a statistical method can be used to identify specific patterns in a person's electromagnetic brain activity and/or specific changes in a person's electromagnetic brain activity. In an example, data from one or more electromagnetic energy sensors can be filtered to remove artifacts before the application of a statistical method. In an example, a filter can be used to remove electromagnetic signals from eye blinks, eye flutters, or other eye movements before the application of a statistical method. In an example, a notch filter can be used as well to remove 60 Hz artifacts caused by AC electrical current. In various examples, one or more filters can be selected from the group consisting of: a high-pass filter, a band-pass filter, a loss-pass filter, an electromyographic activity filter, a 0.5-1 Hz filter, and a 35-70 Hz filter.

In an example, a pattern and/or change in electromagnetic brain activity can be a one-time pattern. In another example, a pattern of electromagnetic brain activity can repeat over time in a rhythmic manner. In an example, a primary statistical method can analyze repeating electromagnetic patterns by analyzing their frequency of repetition, their frequency band or range of repetition, their recurring amplitude, their wave phase, and/or their waveform. In an example repeating patterns and/or waveforms can be analyzed using Fourier Transform methods.

In an example, a primary statistical method for identifying patterns and/or changes in electromagnetic brain activity can comprise finding the mean or average value of data from one or more brain activity channels during a period of time. In an example, a statistical method can comprise identifying a significant change in the mean or average value of data from one or more brain activity channels. In an example, a statistical method can comprise finding the median value of data from one or more brain activity channels during a period of time. In an example, a statistical method can comprise identifying a significant change in the median value of data from one or more brain activity channels. In an example, a statistical method can comprise identifying significant changes in the relative mean or median data values among multiple brain activity channels. In an example, a statistical method can comprise identifying significant changes in mean data values from a first set of sensor locations relative to mean data values from a second set of sensor locations. In an example, a statistical method can comprise identifying significant changes in mean data recorded from a first region of the brain relative to mean data recorded from a second region of the brain.

In an example, a primary statistical method for identifying patterns and/or changes in electromagnetic brain activity can comprise finding the minimum or maximum value of data from one or more brain activity channels during a period of time. In an example, a statistical method can comprise identifying a significant change in the minimum or maximum value of data from one or more brain activity channels. In an example, a statistical method can comprise identifying significant changes in the relative minimum or maximum data values among multiple brain activity channels. In an example, a statistical method can comprise identifying significant changes in minimum or maximum data values from a first set of sensor locations relative to minimum or maximum data values from a second set of sensor locations. In an example, a statistical method can comprise identifying significant changes in minimum or maximum data values recorded from a first region of the brain relative to minimum or maximum data values recorded from a second region of the brain.

In an example, a primary statistical method for identifying patterns and/or changes in electromagnetic brain activity can comprise finding the variance or the standard deviation of data from one or more brain activity channels during a period of time. In an example, a statistical method can comprise identifying a significant change in the variance or the standard deviation of data from one or more brain activity channels. In an example, a statistical method can comprise identifying significant changes in the covariation and/or correlation among data from multiple brain activity channels. In an example, a statistical method can comprise identifying significant changes in the covariation or correlation between data from a first set of sensor locations relative and data from a second set of sensor locations. In an example, a statistical method can comprise identifying significant changes in the covariation or correlation of data values recorded from a first region of the brain and a second region of the brain.

In an example, a primary statistical method for identifying patterns and/or changes in electromagnetic brain activity can comprise finding the amplitude of waveform data from one or more channels during a period of time. In an example, a statistical method can comprise identifying a significant change in the amplitude of waveform data from one or more channels. In an example, a statistical method can comprise identifying significant changes in the relative wave amplitudes from one or more channels. In an example, a statistical method can comprise identifying significant changes in the amplitude of electromagnetic signals recorded from a first region of the brain relative to the amplitude of electromagnetic signals recorded from a second region of the brain.

In an example, a primary statistical method for identifying patterns and/or changes in electromagnetic brain activity can comprise finding the power of waveform brain activity data from one or more channels during a period of time. In an example, a statistical method can comprise identifying a significant change in the power of waveform data from one or more channels. In an example, a statistical method can comprise identifying significant changes in the relative power levels of one or more channels. In an example, a statistical method can comprise identifying significant changes in the power of electromagnetic signals recorded from a first region of the brain relative to the power of electromagnetic signals recorded from a second region of the brain.

In an example, a primary statistical method for identifying patterns and/or changes in electromagnetic brain activity can comprise finding a frequency or a frequency band of waveform and/or rhythmic brain activity data from one or more channels which repeats over time. In an example, Fourier Transform methods can be used to find a frequency or a frequency band of waveform and/or rhythmic data which repeats over time. In an example, a statistical method can comprise decomposing a complex waveform into a combination of simpler waveforms which each repeat at a different frequency or within a different frequency band. In an example, Fourier Transform methods can be used to decomposing a complex waveform into a combination of simpler waveforms which each repeat at a different frequency or within a different frequency band.

In an example, a primary statistical method for identifying patterns and/or changes in electromagnetic brain activity can comprise identifying significant changes in the amplitude, power level, phase, frequency, covariation, entropy, and/or oscillation of waveform data from one or more channels. In an example, a statistical method can comprise identifying significant changes in the amplitude, power level, phase, frequency, covariation, entropy, and/or oscillation of waveform data within a selected frequency band. In an example, a statistical method can comprise identifying significant changes in the relative amplitudes, power levels, phases, frequencies, covariations, entropies, and/or oscillations of waveform data among different frequency bands. In various examples, these significant changes can be identified using Fourier Transform methods.

In an example, brainwaves or other rhythmic, cyclical, and/or repeating electromagnetic signals associated with brain activity can be measured and analyzed using one or more clinical frequency bands. In an example, complex repeating waveform patterns can be decomposed and identified as a combination of multiple, simpler repeating wave patterns, wherein each simpler wave pattern repeats within a selected clinical frequency band. In an example, brainwaves can be decomposed and analyzed using Fourier Transformation methods. In an example, brainwaves can be measured and analyzed using a subset and/or combination of five clinical frequency bands: Delta, Theta, Alpha, Beta, and Gamma. In an example, a method can analyze changes in brainwaves in a single frequency band, changes in brainwaves in multiple frequency bands, or changes in brainwaves in a first frequency band relative to those in a second frequency band.

In an example, Delta brainwaves can be measured and analyzed within a frequency band of 1 to 4 Hz. In various examples, Delta brainwaves or other rhythmic, cyclical, and/or repeating electromagnetic signals associated with brain activity can be measured and analyzed within a frequency band selected from the group consisting of: 0.5 -3.5 Hz, 0.5-4 Hz, 1-3 Hz, 1-4 Hz, and 2-4 Hz. In an example, a method can track a decrease or increase in the relative power of brainwaves in the Delta band. In an example, a method can track a frequency shift within the Delta frequency band. In an example, a method can track a change in wave shape for brainwaves in the Delta frequency band. In an example, a method can track a change in which brain regions originate or modify brainwaves within the Delta frequency band. In an example, a method can track a change in brainwave activity within the Delta band from the anterior vs. posterior areas of a person's brain. In an example, a method can track a change in brainwave activity within the Delta band for a particular brain lobe or organelle. In an example, a method can track a change in brainwave activity within the Delta band as measured from a specific sensor site, a specific sensor channel, and/or a specific montage of channels.

In an example, Theta brainwaves can be measured and analyzed within a frequency band of 4 to 8 Hz. In various examples, Theta brainwaves or other rhythmic, cyclical, and/or repeating electromagnetic signals associated with brain activity can be measured and analyzed within a frequency band selected from the group consisting of: 3.5-7 Hz, 3-7 Hz, 4-7 Hz, 4-7.5 Hz, 4-8 Hz, and 5-7 Hz. In an example, a method can track changes in the power of brainwaves in the Theta band. In an example, a method can track a frequency shift within the Theta band. In an example, a method can track changes in wave shape for brainwaves in the Theta band. In an example, a method can track a change in which brain regions originate or modify brainwaves within the Theta band. In an example, a method can track a change in brainwave activity within the Theta band as measured from a specific sensor site, a specific sensor channel, and/or a specific montage of channels.

In an example, Alpha brainwaves can be measured and analyzed within a frequency band of 7 to 14 Hz. In various examples, Alpha brainwaves or other rhythmic, cyclical, and/or repeating electromagnetic signals associated with brain activity can be measured and analyzed within a frequency band selected from the group consisting of: 7-13 Hz, 7-14 Hz, 8-12 Hz, 8-13 Hz, 7-11 Hz, 8-10 Hz, and 8-10 Hz. In an example, a method can track an increase or decrease in the relative power of brainwaves in the Alpha band. In an example, a method can track a downward or upward shift in the frequency of brainwaves within the Alpha band. In an example, a method can track a change in wave shape for brainwaves in the Alpha frequency band. In an example, a method can track a change in which brain regions originate or modify brainwaves within the Alpha frequency band. In an example, a method can track a change in brainwave activity within the Alpha band on one side of a person's brain relative to the other side. In an example, a method can track a change in brainwave activity within the Alpha band in a particular brain lobe or organelle. In an example, a method can track a change in brainwave activity within the Alpha band as measured from a specific sensor site, a specific sensor channel, and/or a specific montage of channels.

In an example, Beta brainwaves can be measured and analyzed within a frequency band of 12 to 30 Hz. In various examples, Beta brainwaves or other rhythmic, cyclical, and/or repeating electromagnetic signals associated with brain activity can be measured and analyzed within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. In an example, specific patterns or trends in brainwaves in the Beta frequency band can be statistically identified.

In an example, Gamma brainwaves can be measured and analyzed within a frequency band of 30 to 100 Hz. In various examples, Gamma brainwaves or other rhythmic, cyclical, and/or repeating electromagnetic signals associated with brain activity can be measured and analyzed within a frequency band selected from the group consisting of: 30-100 Hz, 35-100 Hz, 40-100 Hz, and greater than 30 Hz. In an example, specific patterns or trends in brainwaves in the Gamma frequency band can be statistically identified.

In an example, a primary statistical method can employ multivariate analysis of electromagnetic brainwave activity in the Delta, Theta, and Alpha frequency bands to identify patterns. In an example, a primary statistical method can comprise calculating an arithmetic function, or a change in an arithmetic function, of the different power levels in multiple frequency bands. In an example, a primary statistical method can comprise a difference, or a change in a difference, between power levels in different frequency bands. In an example, a primary statistical method can comprise a ratio, or a change in a ratio, of power levels in different frequency bands. In an example, a primary statistical method can comprise a sum, or a change in a sum, of power levels in different frequency bands. In an example, a primary statistical method can comprise a product, or a change in a product, of power levels in different frequency bands.

In various examples, specific patterns of electromagnetic brain activity can be analyzed and identified using one or more methods selected from the group consisting of: ANOVA or MANOVA; artificial neural network; auto-regression; Bonferroni analysis; Carlavian curve analysis; centroid analysis; chi-squared analysis; cluster analysis and grouping; decision tree or random forest analysis; Discrete Fourier transform (DFT), Fast Fourier Transform (FFT), or other Fourier Transform methods; factor analysis; feature vector analysis; fuzzy logic model; Gaussian model; hidden Markov model, input-output hidden Markov model, or other Markov model; inter-band mean; inter-band ratio; inter-channel mean; inter-channel ratio; inter-montage mean; inter-montage ratio; Kalman filter; kernel estimation; linear discriminant analysis; linear transform; logit model; machine learning; mean power; mean; median; multi-band covariance analysis; multi-channel covariance analysis; multivariate linear regression or multivariate least squares estimation; multivariate logit or other multivariate parametric classifiers; naïve Bayes classifier, trained Bayes classifier, dynamic Bayesian network, or other Bayesian methods; non-linear programming; pattern recognition; power spectral density or other power spectrum analysis; principal components analysis; probit model; support vector machine; time-series model; T-test; variance, covariance, or correlation; waveform identification; multi-resolution wavelet analysis or other wavelet analysis; whole band power; support vector machine; and Z-scores or other data normalization method.

In an example, a power source for this device can comprise a rechargeable battery. In an example, a power source can be selected from the group consisting of: a rechargeable or replaceable battery; an energy harvesting member which harvests, transduces, or generates energy from body motion or kinetic energy, body thermal energy, or body biochemical energy; an energy harvesting member which harvests, transduces, or generates energy from ambient light energy or ambient electromagnetic energy.

In an example, a data processing unit can process data from one or more electromagnetic energy sensors. In an example a data processing unit can be a microchip, circuit board, CPU, and/or miniature computer. In an example, a data transmitter and/or receiver can be a wireless data transmitter and/or receiver. In an example, data transmitter and/or receiver can be in wireless communication with a remote computer, a handheld electronic device, a separate wearable device, a separate array of wearable sensors, a communication network tower, a satellite, a home control system, and/or an implantable medical device.

I claim:

1. EEG glasses or other electroencephalographic eyewear comprising:
an eyewear frame, wherein the eyewear frame further comprises a frontpiece which is configured to span the front of a person's head, a first sidepiece which is configured to span from a first ear to the frontpiece, and a second sidepiece which is configured to span from a second ear to the frontpiece;
an arcuate protrusion which is part of, or attached to, a selected sidepiece;
an electrode or other electromagnetic energy sensor on the protrusion which collects data concerning brain activity;
an energy source;
a data processor; and
a data transmitter and/or receiver.

2. The EEG glasses or other electroencephalographic eyewear in claim 1 wherein there is a spring between the protrusion and the selected sidepiece.

3. The EEG glasses or other electroencephalographic eyewear in claim 1 wherein one end of the protrusion is attached to the sidepiece and the other end of the protrusion is free to extend out from the sidepiece.

4. The EEG glasses or other electroencephalographic eyewear in claim 3 wherein a posterior end of the protrusion is attached to the sidepiece and an anterior end of the protrusion is free to extend out from the sidepiece.

5. The EEG glasses or other electroencephalographic eyewear in claim 1 wherein both ends of the protrusion are attached to the sidepiece.

6. The EEG glasses or other electroencephalographic eyewear in claim 5 wherein the protrusion has a first configuration which extends a first distance out from the selected sidepiece toward a person's head, wherein the protrusion has a second configuration which extends a second distance out from the selected sidepiece toward the person's head, and wherein the second distance is greater than the first distance.

7. The EEG glasses or other electroencephalographic eyewear in claim 6 wherein the protrusion is changed from the first configuration to the second configuration by moving one or both of the ends of the protrusion closer to each other, causing the protrusion to bulge out towards toward the person's head.

8. The EEG glasses or other electroencephalographic eyewear in claim 7 wherein one or both of the ends of the protrusion slide along the sidepiece.

9. The EEG glasses or other electroencephalographic eyewear in claim 8 wherein one or both of the ends of the protrusion slide along a track or channel on the sidepiece.

10. EEG glasses or other electroencephalographic eyewear comprising:
an eyewear frame which further comprises a front section which is configured to span the front of a person's head, a first side section which is configured to span from a first ear to the front section, and a second side section which is configured to span from a second ear to the front section;
an arcuate tensile protrusion which is part of, or attached to, a selected side section;
an electromagnetic energy sensor which collects data concerning electromagnetic brain activity, wherein the protrusion is configured to hold the electromagnetic energy sensor on the person's head;
an energy source;
a data processor; and
a data transmitter and/or receiver.

11. The EEG glasses or other electroencephalographic eyewear in claim 10 wherein there is a spring between the protrusion and the selected side section.

12. The EEG glasses or other electroencephalographic eyewear in claim 10 wherein one end of the protrusion is attached to the side section and the other end of the protrusion is free to extend out from the side section.

13. The EEG glasses or other electroencephalographic eyewear in claim 12 wherein a posterior end of the protrusion is attached to the side section and an anterior end of the protrusion is free to extend out from the side section.

14. The EEG glasses or other electroencephalographic eyewear in claim 10 wherein both ends of the protrusion are attached to the side section.

15. The EEG glasses or other electroencephalographic eyewear in claim 14 wherein the protrusion has a first configuration which extends a first distance out from the selected side section toward a person's head, wherein the protrusion has a second configuration which extends a second distance out from the selected side section toward the person's head, and wherein the second distance is greater than the first distance.

16. The EEG glasses or other electroencephalographic eyewear in claim 15 wherein the protrusion is changed from the first configuration to the second configuration by moving one or both of the ends of the protrusion closer to each other, causing the protrusion to bulge out towards toward the person's head.

17. The EEG glasses or other electroencephalographic eyewear in claim 16 wherein one or both of the ends of the protrusion slide along the side section.

18. The EEG glasses or other electroencephalographic eyewear in claim 17 wherein one or both of the ends of the protrusion slide along a track or channel on the side section.
